(19) United States
(12) Patent Application Publication
(10) Pub. No.: US 2023/0133393 A1
(43) Pub. Date: May 4, 2023

(54) DIBORIDE MICROPATTERNED SURFACES FOR CELL CULTURE

(71) Applicant: University of Houston System, Houston, TX (US)

(72) Inventors: Fatima MERCHANT, Houston, TX (US); Wanda WOSIK, Houston, TX (US); Jefferson FRIGUGLIGETTI, Houston, TX (US)

(73) Assignee: University of Houston System, Houston, TX (US)

(21) Appl. No.: 17/905,088

(22) PCT Filed: Feb. 26, 2021
(86) PCT No.: PCT/US2021/019933

§ 371 Date: Aug. 26, 2022

Related U.S. Application Data
(60) Provisional application No. 62/982,449, filed on Feb. 27, 2020.

(51) Int. Cl. C12M 1/12; C12M 1/00

(52) U.S. Cl. CPC C12M 25/14; C12M 23/20

(57) ABSTRACT

The present disclosure relates to a micropatterned substrate that combines Si and TiB₂, promoting preferential and selective cell growth behavior via substrate-mediated protein adsorption. The combination of Si and TiB₂, differing in material stiffness, hardness, roughness, wettability and surface charges, is amenable to microfabrication processes and supports extended 2D and 3D cell culture. While versatile in the variety of customizable geometric patterns, the micropatterned substrate is a particularly appropriate platform for viable tissue culture.

### PRIORITY CLAIM

This application is a national phase application under 35 U.S.C. § 371 of International Application No. PCT/US2021/019933, filed Feb. 26, 2021, which claims benefit of priority to U.S. Provisional Application Ser. No. 62/982,449, filed Feb. 27, 2020, the entire contents of each of which are hereby incorporated by reference.

### BACKGROUND

### I. Field

The present disclosure relates to the fields of photolithography, material science, tissue engineering, cell biology, and chemistry. More particularly, the disclosure relates to improved methods and compositions for micropatterned surfaces and their use as cell culture substrates (two-dimensional (2D) and three-dimensional (31)) in vitro tissue culture system).

### II. Related Art

The past few decades have seen a tremendous growth in the development of biomaterials [1] and micro/nano-technologies for micropatterning [2-5]. Conventional optical photolithography, one of the earliest methods used in micropatterning approaches, allows transfer of geometric patterns from a photomask to a substrate such as glass or silicon (Si) wafer, with light exposure [4]. Patterned areas are typically functionalized for cell-type dependent adhesion and growth via manipulation of surface hydrophobicity or chemical modifications, for the presentation of adhesion peptides/proteins. Alternatively, soft lithography techniques utilize biocompatible materials such as polydimethylsiloxane pattern molding, to stamp bioactive molecules or hydrogels to a target surface. While incorporation of bioactive molecules is relatively straightforward in soft lithography, complex surface modifications are required to introduce specific ligands or proteins in conventional photolithography [6].

Si wafers, used in microelectronics for their tunable semiconductor properties, provide an ideal background substrate material for lithography based micropatterning, as they are atomically flat and amenable to microfabrication processes [7, 8]. Si has also been widely used for biological purposes [9-11]. Similarly, Titanium (Ti) and its alloys are also heavily used materials in biomedical applications [12], offering biocompatibility and resistance to corrosion, facilitated by titanium dioxide (TiO₂) [13] that naturally grows on Ti (in air or thermal oxidation) or can be surface deposited [14]. Additionally, reinforcements such as boron [15], a hard, corrosion resistive, biocompatible and inert element, [15, 16], have been used to enhance the mechanical properties of titanium [17, 18]. Boron is one of the main microelements in the human body and plays an important role in the formation and functioning of bone tissue [19, 20]. Accordingly, boron has been used as an osteoinductive agent in various medical materials [21, 22], increasing material biocompatibility and bioactivity due to the formation of B—OH bonds [23, 24]. Notably, Boron-doped TiO₂ coatings exhibited hydrophilic characteristics and improved osteoblast adhesion [25], whereas B-doped TiO₂ particles presented high antibacterial activity [26]. Similarly, titanium diboride (TiB₂), through processes such as boronizing, has growing applications in biomedical sciences, due in part, to its mechanical hardness, stability and wear-resistance [18, 27-32]. Other diborides of the 4^{th} group transition metals Zr and Hf have similar characteristics including mechanical [171], electrical, chemical [172], and thermodynamic [173] properties. These borides show very high melting temperature at their respective congruent compositions and they are ceramic materials with high electrical conductivity and exceptional hardness [174]. The inventors studied TiB₂, ZrB₂ and HfB₂ for their electrical properties such as high work function values and conductivity, as well as for thermodynamic and structural stability important for applications in microelectronics [175-177]. Hf was reported to have similar biocompatibility as Ti when tested via tissue response after implantation into bone and around the surrounding tissue [178]. The addition of Zr and Hf to metallic alloys such as Ti—Nb [179-181] also has shown biocompatibility including applications of shape memory devices [182]. These results show the same behavior of ZrB₂ and HfB₂ as TiB₂ during cellular culture with no signs of surface degradation nor corrosion thus indicating their chemical inactivity. Thus, the present disclosure relates to the use of these materials for micropatterning in tissue culture applications.

Micropatterning has become a standard in biomaterials engineering and is used to study cell-biomaterial interactions and phenomena such as cellular orientation, cytoskeleton rearrangement, cell differentiation and migration [33]. Cellular growth, alignment and orientation on micropatterned surfaces is dependent on properties of the substrate material and cell-type, and further directed by the geometry, topography and the material pattern dimensions used [34, 35]. Cells not only respond to mechanical cues [36] that strongly depend on properties of the substrate materials, but also align and orient in response to the shapes and dimensions of the patterns [37]. Cell adhesion to micropatterned substrates can be further customized by specific chemical functionalization, creating bio-passive or bio-active patterns that improve biomaterial-cell interactions; adhesive proteins adsorbed from supplemented culture media for example [38-41], with protein density and conformation shown to determine cellular behavior [42]. This initial interaction mediates cell attachment and spreading, as well as later events such as proliferation, extracellular matrix (ECM) establishment and reorganization. Importantly, both soluble and immobilized ECM components control the availability and presentation of growth factors which are major regulators of cell behavior [43]. Furthermore, such cellular responses are predictive of cell fate and are the founding basis of the recent explosion in micropatterned device-based applications in cell culture and/or regenerative medicine [5, 34, 41, 44-47].

### SUMMARY

This disclosure concerns a composition comprising a patterned surface, said patterned surface comprising (a) a silicon-containing substrate (Si/SiO₂); and (b) Transition metal diboride from the 4^{th} group TiB₂, ZrB₂, or HfB₂ patterned on said silicon substrate, wherein said patterned surface comprises both silicon and the diboride exposed portions. The patterned surface further may be exposed to one or more biological molecules, and thereby comprises of adsorbed biological molecules, such as heparin, fibroblast growth factor (FGF), insulin-like growth factor-1 (IGF-1), vascular endothelial growth factor (VEGF), endothelial growth factor (EGF), and/or any protein with a heparin-binding domain. The one or more biological molecules may also comprise endothelial cell growth supplement (ECGS), fetal bovine serum (FBS) and heparin, and/or heparin binding proteins, fetal bovine serum (FBS) and heparin.

The patterned surface may comprise one or more diboride exposed zones surrounded by exposed silicon regions, such as 1, 2, 3, 4, 5, 6, 7, 8, 9, 10, 15, 20, 25, 30, 35, 40, 45, 50 75, 100, 150, 200, 250, 500, 750, 1000, 2000, 3000, 4000, 5000, 7500 or 10,000 the diboride exposed zones. The one or more diboride zones may comprise zones of 5 to 1000 μM, such as in the form of lines, circles, squares, rectangles, ovals, and/or any geometric shapes. The patterned surface may enable a 3D microenvironment via cell aggregation. The patterned surface may be located in a microwell, on a slide, chip or wafer, tissue culture flasks, and/or any other conventional tissue culture containers. The patterned surface may comprise ECGS+heparin, FBS+heparin, FBS+ECGS+heparin, and/or FBS+heparin+any heparin binding protein.

Also provided is a method for capturing and/or culturing a cell comprising contacting a cell or cell-containing composition with a composition as defined herein. The cells may be endothelial cells, (e.g., HUVECs), breast cancer cells, ovarian cancer cells (e.g., SKOV3, OVCAR3), mesenchymal stem cells (MSCs), any cells of epithelial and/or endothelial lineage and mesodermal lineage, and non-aggressive and/or aggressive cancer cells, and their combinations (i.e., co-culture of different cell types). The method may further comprise measuring a functional, surface or structural parameter of cell biology. The functional, surface or structural parameter may be growth, migration, division, gene expression, surface biomarkers, biomechanical forces, viability, microskeletal state, oxidative respiration, metastatic potential, apoptosis, secretome, and/or transcriptome. The method may further comprise treating said cell with a drug, biologic, light, heat or radiation. The method may further comprise measuring said functional, surface or structural parameter of cell biology a second time.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIGS. 1A-D: Visualization of micropatterned substrates using a variety of imaging methods for surface characterization. (FIG. 1A) Panel of six substrates designs (100 μm scale bar): Unpatterned Silicon (Si), Unpatterned Titanium diboride (TiB₂), Si with TiB₂ circle patterns of 200 μm diameter, Si with TiB₂ circle patterns ranging in diameter from 200-600 μm, Si with TiB₂ circle patterns of diameter 100 μm and lines of widths 5-20 μm, and Si with TiB₂ circle patterns of diameter 100 μm and lines of widths 5-10 μm. (FIG. 1B) SEM images of the micropatterned substrate at two different magnifications, (FIG. 1C) TEM cross-section of as-deposited TiB₂ on Si, with inset SAED pattern indicating amorphous structure of TiB₂ layer. (FIG. 1D) AFM of patterned substrate. 3D height data of an interface area Si—TiB₂, thickness of TiB₂ layers is about 40 nm, high-resolution topography of an Si background (height range 0-1.0 nm), and high-resolution topography of a TiB₂ pattern (height range 0-1.6 nm). The same patterns were created on ZrB₂ and HfB₂ layers deposited by electron beam evaporation on the silicon substrates.

FIGS. 2A-F: Quantitative characterization of surface, roughness, hardness, composition and charge of the micropatterned substrates. (FIG. 2A) Histograms of Roughness (Rq) calculated with AFM height data for Si (1.6±0.1 Å, blue) and TiB₂ (2.6±0.2 Å, orange). (FIG. 2B) Nanoindentation recorded TiB₂ hardness at 14 GPa and Young's Modulus at 200 GPa compared to values of 10 GPa and 150 GPa, for Si. (FIG. 2C) XPS indicates presence of thin oxides, such as B₂O₃ at 192.6 eV, and Ti—O—B at 523 eV, at the top surface layer (only TiO₂ peak shown) and pure stoichiometric TiB₂ composition in the bulk of the boride films. (FIG. 2D) XPS depth profile for as deposited TiB₂ layer. (FIG. 2E) EDX spectra Si peak is lower in TiB₂ patterns. TiB₂ peak is detected on the patterns but not on the Si background. (FIG. 2F) In DI water, open circuit potential values for TiB₂ are less negative relative to both n- and p-type Si.

FIG. 3: Open Circuit Potential for Si and TiB₂ in the presence of ECGS and heparin. Open circuit potential values for TiB₂ are less negative relative to both n- and p-type Si.

FIGS. 4A-E: Characterization of surface topography and composition in growth factor supplemented culture media. (FIG. 4A) AFM images of Si—TiB₂ substrates in ECGS and heparin. 3D height profile of Si—TiB₂ interface and surface topography of Si and TiB₂ areas. (FIG. 4B) Histogram of surface roughness for Si and TiB₂ in the presence of ECGS and heparin. (FIG. 4C) XPS profile for Si and TiB₂ in the presence of ECGS and heparin. (FIG. 4D) Bearing and particle analysis for height of adsorbed protein on Si and TiB₂ in ECGS without heparin. (FIG. 4E) Bearing and particle analysis for height of adsorbed protein on Si and TiB₂ in ECGS with heparin. In inset images in FIG. 4D and FIG. 4E, height thresholds computed from dry control substrates, were applied to highlight the protein features (shown in cyan) while rejecting substrate features.

FIG. 5: Cellular patterning of HUVECS and MSCs on micropatterned substrates with different media supplements. Images of MSCs at 24-48 hours are shown in the left panel top row, and the bottom row shows the same area following over a week in culture (scale 200 μm). MSCs were cultured on micropatterned Si—TiB₂ (and similarly on ZrB₂ or HfB₂) substrates in media (a) without ECGS and heparin, (b) with ECGS and without heparin, (c) with heparin and without ECGS and (d) with ECGS and heparin. Images of HUVECs cultured on micropatterned Si—TiB₂ substrates in media without ECGS and heparin for a period of 1 to 11 days (top right panel, scale 200 μm), and images of HUVECs cultured in media with ECGS and heparin for a period of 4 to 17 days (bottom, scale 150 μm). Cell counts of MSCs and HUVECs on Si—TiB₂ in supplement-free and supplemented media over a nine-day period are presented in the bottom left panel.

FIGS. 6A-E: Visualization of viability, biomarker expression, and alignment to micropatterns in HUVECs. (FIG. 6A) Fluorescence images of HUVECs stained with viability dye, Acridine Orange, growing on micropatterned substrates with varying geometric patterns of circles and lines (rows 1-3). Images in column 1 are at 4× magnification (scale is 150 μm), with selected areas (colored boxes) in the low magnification images shown at a higher magnification of 20× (columns 2-4, scale is 50 μm). (FIG. 6B) HUVEC cell phenotype immunofluorescence staining for the platelet endothelial cell adhesion molecule 1 (PECAM-1, also known as CD31). (FIG. 6C) HUVEC cell structural immunofluorescence staining for actin (cytoskeleton) and vinculin (focal adhesions). Nuclei are stained blue with DAPI. (FIG. 6D) Plot of HUVEC shape (elongation) on micropatterned substrates with circle and line patterns over a two-week period (scale 150 μm). Top to bottom in sample key correlates to left to right in graph. (FIG. 6E) Quantitative analysis orientation on-line and circle patterns. Top to bottom in sample key correlates to left to right in graph.

FIGS. 7A-E: Morphological, viability and biomarker assessment in MSC 3D aggregates. (FIG. 7A). An XY- and Y-maximum intensity projection generated from confocal z-stacks of the DAPI stained nuclei of an MSC 3D aggregate on a TiB₂ circular micropattern, and a plot of aggregate size (diameter and thickness) versus the pattern size (diameter of circle patterns). (FIG. 7B) Maximum intensity projection generated from confocal z-stacks of an MSC 3D aggregate on a 300 μm diameter circle pattern stained for viability with Acridine Orange (green, live) and Propidium Iodide (red, dead). (FIG. 7C) Maximum intensity projection generated from confocal z-stacks of an MSC 3D aggregate on a 300 μm diameter circle pattern, stained for F-actin (green), nucleus (DAPI, blue), and CD105 (red). (FIG. 7D) Maximum intensity orthogonal projections generated from confocal z-stacks of an MSC 3D aggregate on a 600 μm diameter circle pattern, stained for F-actin (green), nucleus (DAPI, blue), and n-cadherin (red). (FIG. 7E) Individual z-slices for the image in FIG. 7D with grey arrows showing clustering of N-cadherin in aggregate vs homogenous staining in cells at pattern boundary (scale 100 μm).

FIGS. 8A-D: RNA-sequencing Transcriptome Analysis. (FIG. 8A) Venn diagram showing the overlap between the lists of expressed genes in HUVECs grown on conventional tissue culture flasks (Plastic) and the micropatterned TiB₂ substrate (TiB₂). (FIG. 8B) Venn diagram showing the overlap between the lists of expressed genes in MSCs grown on conventional tissue culture flasks (Plastic) and the micropatterned TiB₂ (TiB₂) substrate. (FIG. 8C) Table containing the list of PANTHER pathways identified as over/under-represented when comparing the lists of genes expressed in the studied conditions. (FIG. 8D) Cumulative distribution analyses of the differentially expressed genes in HUVEC cells grown on TiB₂ substrates, the graphs display fold-change distributions of two enriched processes: mitochondrial and NADH activities (Fold-change >±1.5; FDR<0.05).

FIGS. 9A-C: EOC cell line SKOV3 grow into 3D aggregates on micropatterned Si—TiB₂ substrates. (FIG. 9A) Day 3, and (FIG. 9B) Day 5 (100 μm scale): (FIG. 9C) Confocal images of SKOV3 3D aggregates on substrate stained with Acridine Orange/Propidium Iodide viability dyes. Image at 4× (200 μm scale) and 20× (100 μm scale) for patterns of diameter 250 and 500 μm.

FIGS. 10A-C: Cell growth on micropatterned Si—TiB₂ substrates. (FIG. 10A) Panel of six substrates (100 μm scale): Unpatterned (blank) Si, Si with homogenous TiB₂ layer, remaining 4 substrates are Si with varying TiB₂ circle and line patterns. (FIG. 10B) OVCAR3 cells preferentially grow on the TiB₂ micropatterns, in monolayers and fail to form 3D aggregates. (FIG. 10C) SKOV3 cells grow in monolayers on unpatterned TiB₂ (left) and self-assemble into 3D aggregates around days 3-5 (right).

FIGS. 11A-B: Formation inter-aggregate “cellular bridges”. (FIG. 11A) SKOV3 cells grow into 3D aggregates around days 3-5. Formation of cellular bridges is seen occurring across two or more aggregates between 4-6 days (white arrows, 100 μm scale). (FIG. 11B) Immunofluorescence images of DAPI and F-actin at 4×, 20×, and 60× showing multicellular composition of bridges across 3D aggregates.

FIGS. 12A-B: RNA-seq Analysis. (FIG. 12A) Venn diagram. (FIG. 12B) GSEA Top Pathways

FIGS. 13A-E: Effect of SAHA on SKOV3. (FIGS. 13A-B) Untreated Day 7 and Day 9. Growth of cellular bridges is seen (red arrows, 100 μm scale). (FIG. 13C) Day 7 image showing compact 3D aggregate formation and cellular bridges (blue arrows). Aggregates were treated with 304 SAHA for 48 hours. (FIG. 13D) Images taken on Day 9 after SAHA treatment show disassociation of cells resulting in reduction in size of aggregates and loss of cellular bridges (blue arrows). (FIG. 13E) Plot of aggregate depth compared to controls (mean±SEM.; n=3; *p<0.001, using paired t-test) show a ˜50% reduction in size post-treatment with SAHA).

FIGS. 14A-H: Co-culture of MSCs and HUVECs. Images of monocultures and co-cultures of MSCs and HUVECs. FIGS. 6A-C show HUVECs and FIGS. 6D-F show MSCs. Lipophilic membrane dyes are used to track HUVECs (PKH67, green) and MSCs (Cell Vue,red). Phase contrast scale 20 μm, fluorescence scale 100 μm). G-H show aggregate co-cultured MSCs and HUVECs. Phase contrast scale 200 μm, fluorescence scale 100 μm).

SFIGS. 1A-B: MSC growth on the micropatterned substrates in supplemented and supplement free media on different patterns. Top panel: Cellular patterning of MSCs on micropatterned substrates with circle designs. MSCs cultured over a two-week period on micropatterned Si—TiB₂ substrates (scale 200 μm). (SFIG. 1A) Culture media with FBS and antibiotics. (SFIG. 1B) Culture media with FBS, ECGS, heparin and antibiotics. Red arrows indicate formation of MSC 3D aggregates. Bottom panel: Cellular patterning of MSCs on micropatterned substrates with circle and line designs in supplemented media. Image shown for day 6-21. (scale is 200 μm).

SFIGS. 2A-G: Image analysis processing pipeline for determining cell counts on the micropatterned substrates. (SFIG. 2A) Image of substrate prior to cell seeding (scale is 200 μm). (SFIG. 2B) Binary mask image with TiB₂ pattern regions identified in white and Si background in black. (SFIG. 2C) Image of substrate with after seeding MSCs on the substrate in supplement-free media at day 06 (scale is 200 μm). (SFIG. 2D) Binary image delineating cell perimeters in white. (SFIG. 2E) Image showing segmented cell boundaries (red) on the Si background. (SFIG. 2F) Image showing segmented cell boundaries (blue) on the TiB₂ patterns. (SFIG. 2G) Overlay image showing cells growing on the Si background (red) and on the TiB₂ patterns (blue).

SFIG. 3: Cellular patterning of MSCs on micropatterned substrates with circle and line patterns. MSCs cultured in supplemented media over a three-week period on micropatterned Si—TiB₂ substrates with circle and line patterns (scale 500 μm).

SFIG. 4: HUVEC cells were seeded and grown over a two-week period. Substrates were sampled at different time points; days 1, 4, 7, 9, 11 and 13 after seeding on substrates with circle patterns of 450 μm diameter. Cell growth and viability on the patterned substrates were computed by determining the total area of viable cells covering the patterned surfaces. As seen here, HUVECs are viable (retain green fluorescence) until day 13 after seeding. For quantitative analysis, the total area of cells stained green was determined and the ratio of the area of viable cells to the area of the circle pattern (i.e., 0.158 mm²) was computed. As shown in the plot of the percentage area of the pattern covered with viable cells at different days following seeding, significant growth was observed from day 4 until day 7 after seeding (p-value of 0.0002<0.05), following which a reduction in the number of cells is observed on day 11 (p-value of 0.004<0.05), and subsequent maintenance of cell growth at a steady state (days 11-13; p>0.05).

SFIGS. 5A-D: (SFIG. 5A) Transcripts expressed in HUVECs on plastic and on the micropatterned TiB₂ were mapped for GO term of “biological process” using PANTHER, (SFIG. 5B) Transcripts expressed in MSCs on plastic and on the micropatterned TiB₂ were mapped for GO term of “biological process” using PANTHER. (SFIG. 5C) Transcripts expressed in HUVECs on plastic and on the micropatterned TiB₂ were mapped for GO term of “molecular function” using PANTHER, and (SFIG. 5D) Transcripts expressed in MSCs on plastic and on the micropatterned TiB₂ were mapped for GO term of “molecular function” using PANTHER.

SFIGS. 6A-B: Gene function analysis using the PANTHER classification system for HUVECs cultured in plastic tissue cultured flasks coated with 0.2% gelatin versus those cultured on titanium diboride substrates. (SFIG. 6A) Biological Processes and (SFIG. 6B) Molecular Function.

### DETAILED DESCRIPTION

In this study, the inventors introduce and evaluate a novel substrate for cell culture application, using epithelial (HUVECs), mesodermal (MSCs) and ovarian cancer cell lines (SKOV3, OVCAR3) used extensively in tissue engineering and cancer biology research, demonstrating tremendous potential for clinical translation [48-54]. MSCs are self-renewing stem cells that exhibit multipotent differentiation potential and immunomodulatory properties. Moreover, their ease of isolation and ability to expand via in vitro culture have made them attractive therapeutic agents. HUVECs, are a widely accepted model to study the function and pathology of endothelial cells as well as the generation of microfluidic vascular networks in engineered tissue [55]. Importantly, both cell types have also been used extensively to study the effect of substrate surface properties such as roughness, stiffness, electrical charge and chemistry, on patterns of cell behavior including adhesion, shape, alignment and mechanotransduction [14, 56-58]. Cell-type specific behaviors such as contact guidance and alignment, durotaxis and aggregation, have been observed for both HUVECs and MSCs respectively.

The Ovarian Cancer Cell Line Panel (OCCP) highlights the clinical importance of understanding and characterizing the differences of the morphological subtypes via in vitro studies [228]. Ovarian cancer cells lines expressing traditional epithelial-like morphology are cultured from tissue and are closely adjoined by specialized membrane structures such as tight, adherent, and gap junctions, while cell lines that express a mesenchymal-like morphology are extracted from swollen compromised tissue such as in ascites or pleural effusions and form an organized cell layer.

Genomic profiles for epithelial cell lines indicate that both OVCAR3 and SKOV3 are have been examined. The main differences between both cell lines are their morphologies and their different levels of invasiveness [204]. OVCAR3 has a rounded shape and tends to present less invasion potential, while SKOV3 has a more spindle-like shape with greater invasion potential. Serous tumors commonly arise in the epithelium of the fallopian tube fimbria and subsequently present as apparent ovarian tumors after implantation in the ovary and present at an advanced stage, are fast-growing, and spread throughout the peritoneal cavity. While studies have shown the genetic mutation of OVCAR3 tumors is distinct from SKOV3 tumors, their differences in invasiveness behavior are poorly defined and thus highlight the importance of understanding how the treatment of tumors with therapeutic options might influence invasive and migratory behavior.

The inventors also describe a novel micropatterned substrate that combines Si and diborides of transition metals from the 4^{th} periodic group (TiB₂, ZrB₂, or HfB₂) promoting preferential and selective cell growth behavior via substrate mediated protein adsorption. The novel combination of Si and these borides, differing in material stiffness, hardness, roughness, wettability and surface charges, is amenable to microfabrication processes and supports extended cell culture. While versatile in the variety of customizable geometric patterns, the micropatterned substrate is an appropriate platform for viable tissue culture. HUVECS, MSCs and SKOV3 demonstrate stable and robust cellular profiles as indicated by viable proliferation, biomarker expression and transcriptome analysis. Importantly, endothelial cell growth supplement (ECGS) and heparin play a dominant role in establishing specific HUVEC and MSC cell growth. In their absence, cells exhibit preferential attachment to the diborides (TiB₂ ZrB₂ and HfB₂) patterns versus Si (i.e., more cells attach to micropatterns versus the Si background), whereas highly selective growth (i.e., cells attach only to micropatterns) is observed in supplemented media. Similarly, basic fibroblast growth factor (FGF) in the presence of heparin induces pattern specific growth of SKOV3 over a period of 7 days in culture. In line with advanced cell culture techniques, aggregation of MSCs and SKOV3 on the inventors' substrate provides a three-dimensional (3D) culture microenvironment which is crucial in driving essential cellular development, regeneration, and differentiation processes in MSCs [46, 59] and tumorigenesis in SKOV3. Potential substrate applications include coculture of multiple cell types, mesenchymal stem cell differentiation, induced pluripotent stem cell differentiation, adipose stem cell differentiation and high throughput technologies for drug development in 2/3D cancer cultures.

### I. Micropatterning

Micropatterning is the art of miniaturization of patterns. Initially used for electronics, it has recently become a standard in biomaterials engineering and for fundamental research on cellular biology by mean of soft lithography. It generally uses photolithography methods but many techniques have been developed.

In cellular biology, micropatterns can be used to control the geometry of adhesion and substrate rigidity. This tool helped scientists to discover how the environment influences processes such as the orientation of the cell division axis, organelle positioning, cytoskeleton rearrangement cell differentiation and directionality of cell migration. Micropatterns can be made on a wide range of substrates, from glass to polyacrylamide and polydimethylsiloxane (PDMS). The polyacrylamide and PDMS in particular come into handy because they let scientists specifically regulate the stiffness of the substrate, and they allow researchers to measure cellular forces (traction force microscopy). Advanced custom micropatterning allows precise and relatively rapid experiments controlling cell adhesion, cell migration, guidance, 3D confinement and microfabrication of microstructured chips. Using advanced tools, protein patterns can be produced in virtually unlimited numbers (2D/3D shapes and volumes).

Nanopatterning of proteins has been achieved through using top-down lithography techniques. Aerosol micropatterning for biomaterials uses spray microscopic characteristics to obtain semi-random patterns particularly well adapted for biomaterials.

The following terms are defined hereinbelow and apply to the presently disclosure and claims therefor.

The term “culture platform” refers to a substrate comprising a nanotextured surface which is micropatterned with an array of one or more geometric units.

The term “nanotextured” is used interchangeably herein with “nanotopographic features” or “nanotopography” and “nanopatterned”, “nanogrooved”, refer to a nano-scale patterned surface. The term “micropatterned” or “micropattern” as used herein refers to a micron scale pattern on a surface

The term “cell adherent region” is used interchangeably herein with “cell permissive region” and refers to a region to which a cell binds selectively or preferentially relative to other regions of the surface. A cell adherent region is bounded by at least one lesser- or non-adherent region. While it is preferred that a cell non-adherent region does not permit cell adhesion at all, at the minimum, a cell adherent region allows at least 75% more cells to attach to the surface relative to the proportion of cells attaching to the same surface area of a cell non-adherent region.

The term “cell non-adherent region” refers to the surface of the nanotextured or nanopatterned substrate onto which cells do not substantially, or substantially attach. A cell non-adherent region allows no more than 5% of cells to attach to the surface, relative to the proportion of cells attaching to the same surface area of a cell adherent region.

The term “soft-lithography” as used herein refers to a technique commonly known in the art. Soft-lithography uses a patterning device, such as a stamp, a mold or mask, having a transfer surface comprising a well-defined pattern in conjunction with a receptive or conformable material to receive the transferred pattern. Microsized and nanosized structures are formed by material processing involving conformal contact on a molecular scale between the substrate and the transfer surface of the patterning device.

A “patterning device” is intended to be broadly interpreted as referring to a device that can be used to convey a patterned cross-section, corresponding to a pattern that is to be created in a target portion of the substrate.

A “pattern” is intended to mean a pre-determined mark or design, generally a substantially microscale or nanoscale design.

The term “surface” is used interchangeably herein with “substrate” or “scaffold” and should be understood in this connection to mean any suitable carrier material to which the cells are able to attach or adhere (either inherently or following treatment to promote cell adhesion) and which can be nanotextured and micropatterned as described herein. In some embodiments, the substrate is a “biocompatible substrate” as that term is defined herein. In one embodiment, the biocompatible substrate provides the supportive framework that allows cells to attach to it and grow on it. Cultured populations of cells can then be grown on the biocompatible substrate.

The term “tissue” refers to a group or layer of similarly specialized cells which together perform certain special functions. The term “tissue” is also intended to include intact cells, blood, blood preparations such as plasma and serum, bones, joints, muscles, smooth muscles, and organs. The term “tissue-specific” refers to a source or defining characteristic of cells from a specific tissue.

The term “cell culture medium” (also referred to herein as a “culture medium” or “medium”) as referred to herein is a medium for culturing cells, containing nutrients and other factors that maintain cell viability and support cell proliferation. The cell culture medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art.

### II. Materials

In accordance with the present disclosure, the inventors have identified an improved combination of surface features that can be employed in the production of patterned surfaces for us in cell binding and culture. The primary surface components are TiB₂, ZrB₂, and HfB₂ and Si, along with optional biological factors attached to those surface components.

A. TiB₂, ZrB₂, and HfB₂

Diborides of Titanium (TiB₂), Zirconium (ZrB₂), and Hafnium (HfB₂) [183] are extremely hard ceramic which have excellent heat conductivity, oxidation stability and resistance to mechanical erosion. They are also good electrical conductors, so TiB₂ can be used as a cathode protecting material in aluminium melting and can be shaped by electrical discharge machining.

TiB₂ shares some properties with Boron Carbide & Titanium carbide, but many of its properties are superior to those of B4C & TiC such as exceptional hardness at extreme temperature. These borides have similar advantages over borides from other periodic groups such as having the highest values of Elastic Modulus, highest shear module (G), smallest Poisson ratio. Hardness is very high and ranges from 43 to 50 GPa for these borides. Electron work function (EWF) values are high, typically below 5 eV, indicating high chemical stability of atomic bondings in these materials. That changes depending on crystallographic orientation including polycrystalline structures and atomic surface termination (for B EWF-6 eV and for Ti EWF-4.6 eV) [184]. These measurements of work functions in TiB₂ and ZrB₂ based on Kelvin probe and on MOS capacitors confirmed the dependence on crystallization due to annealing and formation a thin surface oxide which increases EWF [175]. All these diborides have phase diagrams showing congruent composition at melting temperatures TiB₂ (3225° C.), ZrB₂ (3247° C.), and HfB₂ (3380° C.) that facilitate stoichiometric evaporation during E-beam deposition. Other advantages include high thermal conductivity (60-120 W/m K), and high electrical conductivity (˜10⁵ S/cm). It is, however, difficult to mold due to high melting temperature and difficult to Sinter due to the high covalent bonding. It also is limited to pressing to small monolithic pieces using of Spark Plasma Sintering.

With respect to chemical stability, TiB₂ is more stable in contact with pure iron than tungsten carbide or silicon nitride. These diborides are prone to oxidation in air at high temperatures [185], and react with selected acids (hydrofluoric, nitric acid and sulfuric acid) and H₂O₂.

TiB₂ does not occur naturally in the earth. Titanium diboride powder can be prepared by a variety of high-temperature methods, such as the direct reactions of titanium or its oxides/hydrides, with elemental boron over 1000° C., carbothermal reduction by thermite reaction of titanium oxide and boron oxide, or hydrogen reduction of boron halides in the presence of the metal or its halides. Among various synthesis routes, electrochemical synthesis and solid-state reactions have been developed to prepare finer titanium diboride in large quantity. An example of solid-state reaction is the borothermic reduction, which can be illustrated by the following reactions:

2TiO₂+B₄C+3C→2TiB₂+4CO (1)

TiO₂+3NaBH₄→TiB₂+2Na(g,l)+NaBO₂+6H₂(g) (2)

The first synthesis route (1), however, cannot produce nanosized powders. Nanocrystalline (5-100 nm) TiB₂ was synthesized using the reaction (2) or the following techniques:
solution phase reaction of NaBH₄ and TiCl₄, followed by annealing the amorphous precursor obtained at 900-1100° C.;
mechanical alloying of a mixture of elemental Ti and B powders;
self-propagating high temperature synthesis process involving addition of varying amounts of NaCl;
milling assisted Self-propagating high temperature synthesis (MA-SHS) solvothermal reaction in benzene of metallic sodium with amorphous boron powder and TiCl₄ at 400° C.:

TiCl₄+2B+4Na→TiB₂+4NaCl

Many TiB₂ applications are inhibited by economic factors, particularly the costs of densifying a high melting point material, and, thanks to a layer of titanium dioxide that forms on the surface of the particles of a powder, it is very resistant to sintering. Admixture of about 10% silicon nitride facilitates the sintering, though sintering without silicon nitride has been demonstrated as well.

Thin films of TiB₂ can be produced by several techniques. The electroplating of TiB₂ layers possess two main advantages compared with physical vapor deposition or chemical vapor deposition: the growing rate of the layer is 200 times higher (up to 5 μm/s) and the inconveniences of covering complex shaped products are dramatically reduced.

Current use of TiB₂ appears to be limited to specialized applications in such areas as impact resistant armor, cutting tools, crucibles, neutron absorbers and wear resistant coatings. TiB₂ is extensively used for e-beam evaporation boats as a crucible. For evaporation of higher melting temperature materials such as TiB₂ or other borides SiC crucibles are used. TiB₂ is an attractive material for the aluminium industry as an inoculant to refine the grain size when casting aluminium alloys, because of its wettability by and low solubility in molten aluminium and good electrical conductivity. Thin films of TiB₂ can be used to provide wear and corrosion resistance to a cheap and/or tough substrate.

B. Silicon

Silicon is a chemical element with the symbol Si and atomic number 14. It is a hard and brittle crystalline solid with a blue-grey metallic lustre; and it is a tetravalent group 4 indirect semiconductor. It is a member of group 14 in the periodic table: carbon is above it; and germanium, tin, and lead are below it. It is relatively unreactive and its melting point of 1414° C. is used to grow single crystals. Silicon is the eighth most common element in the universe by mass, but very rarely occurs as the pure element in the Earth's crust. It is most widely distributed in dusts, sands, planetoids, and planets as various forms of silicon dioxide (silica) or silicates. More than 90% of the Earth's crust is composed of silicate minerals, making silicon the second most abundant element in the Earth's crust (about 28% by mass) after oxygen.

Most silicon is used commercially without being separated, and often with little processing of the natural minerals. Such use includes industrial construction with clays, silica sand, and stone. Silicates are used in Portland cement for mortar and stucco and mixed with silica sand and gravel to make concrete for walkways, foundations, and roads. They are also used in whiteware ceramics such as porcelain, and in traditional quartz-based soda-lime glass and many other specialty glasses. Silicon compounds such as silicon carbide are used as abrasives and components of high-strength ceramics. Silicon is the basis of the widely used synthetic polymers called silicones.

Crystalline bulk silicon is rather inert but becomes more reactive at high temperatures. Like its neighbor aluminium, silicon forms a thin, continuous surface layer of silicon dioxide (SiO₂) that protects the semiconductor from further oxidation. Silicon measurably reacts with the air which increases with temperature and depends on the oxidation ambient. Annealing in nitrogen ambient at high temperatures leads to nitrides formation SiN and Si₃N₄. Silicon reacts with gaseous sulfur at 600° C. and gaseous phosphorus at 1000° C. This oxide layer nevertheless does not prevent reaction with the halogens; fluorine attacks silicon vigorously at room temperature, chlorine does so at about 300° C., and bromine and iodine at about 500° C. Silicon does not react with most aqueous acids, but is oxidized and etched by a mixture of concentrated nitric acid and hydrofluoric acid; it readily dissolves in hot aqueous alkali to form silicates. At high temperatures, silicon also reacts with alkyl halides; this reaction may be catalyzed by copper to directly synthesize organosilicon chlorides as precursors to silicone polymers. Silicon reacts with metals to form silicides. Growth of silicon crystals is typically done by Czochralski method where silicon melt is kept in quartz crucible supported by graphite.

Silicon dioxide (SiO₂), also known as silica, is one of the best-studied compounds, second only to water. Twelve different crystal modifications of silica are known, the most common being α-quartz, a major constituent of many rocks such as granite and sandstone. It also is known to occur in a pure form as rock crystal; impure forms are known as rose quartz, smoky quartz, morion, amethyst, and citrine. Some poorly crystalline forms of quartz are also known, such as chalcedony, chrysoprase, carnelian, agate, onyx, jasper, heliotrope, and flint. Other modifications of silicon dioxide are known in some other minerals such as tridymite and cristobalite, as well as the much less common coesite and stishovite. Biologically generated forms are also known as kieselguhr and diatomaceous earth. Vitreous silicon dioxide is known as tektites, and obsidian, and rarely as lechatelierite. Some synthetic forms are known as keatite and W-silica. Opals are composed of complicated crystalline aggregates of partially hydrated silicon dioxide. One of the most important uses of Si single crystals is the microelectronic industry for fabrication of silicon integraded circuits.

C. Biological Factors

One or more biological factors may be added to the surface of the Si—TiB₂, Si—ZrB₂ or Si—HfB₂ substrates. These factors may provide a binding function for cells which are subsequently added to the patterned substrate, or they may provide signals to the cells once bound, i.e., growth, division, migration, etc.

One contemplated binding factor is heparin, also known as unfractionated heparin (UFH). Heparin is a medication and naturally occurring glycosaminoglycan. As a medication it is used as an anticoagulant (blood thinner). Specifically, it is also used in the treatment of heart attacks and unstable angina. It is given by injection into a vein or under the skin. Other uses include inside test tubes and kidney dialysis machines. A fractionated version of heparin, known as low molecular weight heparin, is also available.

Native heparin is a polymer with a molecular weight ranging from 3 to 30 kDa, although the average molecular weight of most commercial heparin preparations is in the range of 12 to 15 kDa. Heparin is a member of the glycosaminoglycan family of carbohydrates (which includes the closely related molecule heparan sulfate) and consists of a variably sulfated repeating disaccharide unit. The main disaccharide units that occur in heparin are shown below. The most common disaccharide unit is composed of a 2-O-sulfated iduronic acid and 6-O-sulfated, N-sulfated glucosamine, IdoA(2S)-GlcNS(6S). For example, this makes up 85% of heparins from beef lung and about 75% of those from porcine intestinal mucosa.

Not shown below are the rare disaccharides containing a 3-O-sulfated glucosamine (GlcNS(3S,6S)) or a free amine group (GlcNH₃⁺). Under physiological conditions, the ester and amide sulfate groups are deprotonated and attract positively charged counterions to form a heparin salt. Heparin is usually administered in this form as an anticoagulant.

A broad category of biological agent that is of particular utility in accordance with the present disclosure is growth factors. A growth factor is a naturally occurring substance capable of stimulating cellular growth, proliferation, healing, and cellular differentiation. Usually it is a protein or a steroid hormone. Growth factors are important for regulating a variety of cellular processes. Growth factors typically act as signaling molecules between cells. Examples are cytokines and hormones that bind to specific receptors on the surface of their target cells.

They often promote cell differentiation and maturation, which varies between growth factors. For example, epidermal growth factor (EGF) enhances osteogenic differentiation, while fibroblast growth factors (FGF-1-23) and vascular endothelial growth factors (VEGFs) stimulate blood vessel differentiation (angiogenesis). Insulin-like growth factors (IGFs) are stimulated by human growth hormone and promote growth. Others include colony stimulating factors (G-CSF, M-CSF, GM-CSF), ephrins, interleukins, neuregulins, transforming growth factors (TGF-α and -β), and neutrophilins (nerve growth factor, brain-derived neurotrophic factor, neurotrophin-3 and -4).

Biological factors may also include complex biological solutions such as growth serum. For example, fetal bovine serum (FBS) comes from the blood drawn from a bovine fetus via a closed system of collection at the slaughterhouse. Fetal bovine serum is the most widely used serum-supplement for the in vitro cell culture of eukaryotic cells. This is due to it having a very low level of antibodies and containing more growth factors, allowing for versatility in many different cell culture applications. The globular protein, bovine serum albumin (BSA), is a major component of fetal bovine serum. The rich variety of proteins in fetal bovine serum maintains cultured cells in a medium in which they can survive, grow, and divide. FBS is not a fully defined media component, and as such may vary in composition between batches. As a result, serum-free and chemically defined media (CDM) have been developed as a matter of good laboratory practice. The effectiveness of serum-free media is limited, however; many cell lines still require serum in order to grow, and many serum-free media formulations are intended for narrowly defined types of cells. Therefore, fetal serum is still widely used in cell culture.

Endothelial Cell Growth Supplement (ECGS) is a poorly defined supplement that usually consists of acidic-FGF (aFGF), basic-FGF (bFGF), and various attachment factors that serve as signaling molecules. bFGF, or basic fibroblast growth factor, is the active component in ECGS that signals endothelial cell growth. Heparin binds to FGF and promotes ligand formation with the FGFR (FGF-receptor). As such, it is important to add heparin to culture medium in conjunction with FGF. It may be necessary to try different lots of ECGS, since this supplement is undefined and usually contains varying concentrations of bFGF.

### III. Micropatterning/Nanopatterning Methodologies

The micropatterning of substrates is a well-known process where substrates are fabricated by various deposition, annealing, and growth processes and are patterned by photolithography processes which includes deposition of photosensitive resist (PR), alignment of the patterns, developing of PR and etching of a specific unmasked regions.

Electron-beam evaporation is a physical vapor deposition process, or EBPVD, in which an ingot target material in a crucible is bombarded with an electron beam generated by a charged tungsten filament at high voltage in a kV range and under high vacuum. The electron beam causes atoms from the target to transform into the gaseous phase. These atoms are then deposited onto a solid substrate (here Si wafers) to create well controlled layer.

In an EBPVD system, the deposition chamber must be evacuated to a pressure of at least 1×10⁻⁶ Torr to ensure minimum contamination levels incorporated into the film and allow for uniform deposition of the films. Multiple types of evaporation materials and electron guns can be used simultaneously in a single EBPVD system, each having a power from tens to hundreds of kilowatts. Electron beams can be generated by thermionic emission, field electron emission or the anodic arc method. The generated electron beam is accelerated to a high kinetic energy and directed towards the evaporation material. Upon striking the evaporation material, the electrons will lose their energy very rapidly. The kinetic energy of the electrons is converted into other forms of energy through interactions with the evaporation material. The thermal energy that is produced heats up the evaporation material causing it to melt or sublimate. Once temperature and vacuum level are sufficiently high, vapor will result from the melt or solid. The resulting vapor can then be used to coat surfaces. Accelerating voltages can be between 3 and 40 kV. When the accelerating voltage is 20-25 kV and the beam current is a few amperes, 85% of the electron's kinetic energy can be converted into thermal energy. Some of the incident electron energy is lost through the production of X-rays and secondary electron emission.

Depending of the phase diagram of evaporated materials, some refractory carbides like titanium carbide and borides like titanium boride, zirconium boride, and hafnium boride can evaporate without undergoing decomposition in the vapor phase. These compounds are deposited as thin films on a given substrate by direct evaporation from ingot chunks.

The substrate on which the film deposition takes place is cleaned according to proper cleaning recepy, dried and fastened to the substrate holder. The substrate holder is attached to the manipulator shaft. The manipulator shaft moves translationally to adjust the distance between the ingot source and the substrate. The shaft also rotates the substrate at a particular speed so that the film is uniformly deposited on the substrate. Often, focused high-energy electrons from one of the electron guns or infrared light from heater lamps can be used to preheat the substrate. Heating of the substrate allows increased adatom-substrate and adatom-film diffusion by giving the adatoms sufficient energy to overcome kinetic barriers.

The inventors selected e-beam evaporation process for all three diborides as the most reproducible deposition method for high melting temperature compounds with congruent composition identified by their phase diagrams. E-beam does not have in situ cleaning options so surface preparations for the substrate prior deposition and high vacuum have to be strictly followed.

Several deposition systems such as sputtering have the advantage of in situ cleaning by ion bombardent prior film deposition, but the technology of diborides deposition is very difficult. Also, chemical vapor deposition (CVD) methods are very difficult to develop to form very thin layer of compound materials.

Optical lithography processes, also called photolithography (including UV and extreme UV lithography), is a process used in microfabrication to create patterns on thin films or the bulk of a substrate (also called a wafer). It uses light to transfer a geometric pattern from a photomask (also called an optical mask) to a photosensitive (that is, light-sensitive) chemical photoresist on the substrate. A series of chemical treatments then allows to develop, etch the exposure pattern into the material. In complex integrated circuits, a CMOS wafer may go through the lithographic cycle as many as 50 times.

Photolithography shares some fundamental principles with photography in that the pattern in the photoresist etching is created by exposing it to light, either directly (without using a mask) or with a projected image using a photomask. This procedure is comparable to a high precision version of the method used to make integrated circuits or printed circuit boards. This method, with detailed modifications can create extremely small patterns, down to a few tens or even a few nanometers in size. It provides precise control of the shape and size of the objects it creates and can create patterns over an entire surface cost-effectively. Lithography requires extremely clean operating conditions, which is a standard in the cleanroom facilities.

A single iteration of photolithography combines several steps in sequence. Modern cleanrooms use automated, robotic wafer track systems to coordinate the process. The Photolithography process is carried out by the wafer track and stepper/scanner, and the wafer track system and the stepper/scanner are installed side by side. The general steps include cleaning, preparation, photoresist application, exposure and developing, etching and photoresist removal.

To ensure surface cleaneliness prior lithography, necessary for good adhesion of photoresist wet chemical treatment can be used. Then the wafer is initially heated to a temperature sufficient 150° C. for outgassing, i.e., removing moisture that may be present on the wafer surface. A liquid or gaseous “adhesion promoter,” such as Bis(trimethylsilyl)amine (“hexamethyldisilazane”, HMDS), can be used to promote adhesion of the photoresist to the wafer.

The wafer is covered with photoresist by spin coating. Final thickness is also determined by rpm of the spin coater and viscosity of the PR. For very small, dense features (<125 or so nm), lower resist thicknesses (<0.5 microns) are needed to overcome collapse effects at high aspect ratios; typical aspect ratios are <4:1. The photo resist-coated wafer is then prebaked to drive off excess photoresist solvent, typically at 90 to 100° C. for 30 to 60 seconds on a hotplate. A BARC coating (Bottom Anti-Reflectant Coating) may be applied before the photoresist is applied, to avoid reflections from occurring under the photoresist and to improve the photoresist's performance at smaller semiconductor nodes with reflective surfaces.

After prebaking, the photoresist is exposed to a pattern of intense light of specific wavelength determined by the feature sizes. The exposure to light causes a chemical change that allows some of the photoresist to be removed by a special solution, called “developer” by analogy with photographic developer. Positive photoresist, the most common type, becomes soluble in the developer when exposed; with negative photoresist, unexposed regions are soluble in the developer. A post-exposure bake (PEB) can be performed before developing, typically to help reduce standing wave phenomena caused by the destructive and constructive interference patterns of the incident light. In deep ultraviolet lithography, chemically amplified resist (CAR) chemistry is used. This process is much more sensitive to PEB time, temperature, and delay, as most of the “exposure” reaction (creating acid, making the polymer soluble in the basic developer) actually occurs in the PEB.

The developer is delivered using spincoater, much like that used for photoresist deposition. Developers originally often contained sodium hydroxide (NaOH). However, sodium is considered an extremely undesirable contaminant in Si chips fabrication because it degrades the insulating properties of gate oxides (specifically, sodium ions can migrate in and out of the gate, changing the threshold voltage of the transistor and making it harder or easier to turn the transistor on over time). Metal-ion-free developers are used as specified by the PR producers to complete pattering of the PR. Depending on the PR type used one can use “hard-baked” if a non-chemically amplified resist was used, typically at 120 to 180° C. for 20 to 30 minutes. The hard bake, if required solidifies the remaining photoresist, to make a more durable protecting layer in future ion implantation, wet chemical etching, or plasma etching.

In etching, a liquid (“wet”) or plasma (“dry”) chemical agent removes the uppermost layer of the substrate in the areas that are not protected by photoresist. In semiconductor fabrication, dry etching techniques are generally used, as they can be made anisotropic, in order to avoid significant undercutting of the photoresist pattern. This is essential when the width of the features to be defined is similar to or less than the thickness of the material being etched (i.e., when the aspect ratio approaches unity). Wet etch processes are generally isotropic in nature, which is often indispensable for microelectromechanical systems, where suspended structures must be “released” from the underlying layer.

Before etching, the inspection of the pattern created in a photoresist has to be performed, followed by etching process, again inspection and resist removal using “resist stripper” or frequently, acetone. Alternatively, photoresist may be removed by a oxygen plasma known as “plasma ashing”. Final rinsing in acetone, alcohol and deionized water frequently follows wet resist removal.

### IV. Cell Biology Uses for Micropatterned Surfaces

In situ, cells are highly sensitive to geometrical and mechanical constraints from their microenvironment, such as the substrates upon which they grown. Micropatterning of the surfaces of substrates can significantly affect the ability and type of cell growth and behavior. For example, micropatterning can be used to restrict the location and shape of the culture regions. Engineered micropatterns can provide micrometer or nanoscale, soft, 3-dimensional, complex and dynamic microenvironment for individual cells or for multi-cellular arrangements. These micropatterned substrates have the advantage of potentially recapitulating physiological conditions for in vitro cell culture. For example, by manipulating micropattern shapes, cells can be driven to adapt their cytoskeleton architecture to the geometry of their microenvironment, thus causing re-modelling of actin and microtubule networks and adaptation of cell polarity. These modifications further impact cell migration, growth and differentiation.

In particular applications:
Differentiation of stem cells (mesenchymal stem cells, induced pluripotent stem cells, adipose stem cells, embryonic stem cells, other non-embryonic (adult) stem cells, cord blood stem cells and amniotic fluid stem cells) into other cells types such as insulin producing cells, osteoblasts, chondrocytes, etc.
Co-culture of different cell types
Co-culture of different cell types for developing organoids
Drug discovery platform for screening chemical and epigenetic candidate drugs
Cancer stem cell screening and analysis
Cancer metastasis screening and analysis

### V. Examples

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Microfabrication of Patterned Substrates. Micropatterned substrates were fabricated by means of a sequence of well-established processes such as physical deposition by e-beam evaporation, patterning by photolithography, and wet etching. TiB₂ (and ZrB₂, HfB₂) layers ranging in thickness from 30 to 100 nm were deposited by e-beam evaporation from melted 99.9% chunks of these borides on (100) oriented low doped n- and p-type Si wafers cleaned using standard RCA clean [60]. Prior e-beam evaporation, the substrates were dipped in 0.5% hydrofluoric acid (HF) to remove surface oxide, rinsed with deionized (DI) water and dried in N₂. Parameters of the deposition process included e-beam energy 8 keV, base pressure below 3×10⁻⁷ torr, operation pressure maintained below 3×10⁻⁶ torr and the deposition rate of 7-15 Å/sec. Masks with the pattern consisting of arrays of circles with diameter ranging from 100-600 μm and/or lines of variable lengths reaching ˜500 μm and widths varying from 5-50 were made using chromium layers deposited and patterned on glass for the photolithography process. The borides layers were patterned into designed structures using optical lithography in the contact mode and negative photoresist (Futurrex, Franklin, N.J.) for patterns exposure. Wet etching process in 30% hydrogen peroxide was used for TiB₂ (for ZrB₂ and HfB₂ 5% of HF was added) on the deposited films with patterned photoresist acting as a mask. Photoresist was then striped in acetone and rinsed in IPA followed by DI water rinse. The clean wafers were dried using compressed nitrogen (N2) air gun. For experimentation, substrates were cut into small squares or rectangles using a diamond cutter, generally between 20-40 mm² in area. Substrates were sterilized by 30 minutes of immersion in 70% ethanol prior to cell culture.

Material Surface Characterization Protocols. Molecular composition of the micropatterned substrates was determined using the X-ray photoelectron spectroscopy (XPS; Model 5700, Physical Electronics, Inc., Chanhassen, Minn.), operating at a background pressure less than 5×10⁻⁹ torr. The instrument was used to collect photoelectrons, which had been produced using a monochromatic A1-kα x-ray source (1486.6 eV) operated at 350 W. Analyzed area was set to 800 and the collection solid cone and take off angle were 5° and 45° respectively. A pass energy of 11.75 eV caused an energy resolution greater than 0.51 eV. Spectra were obtained in a vacuum of at least 5×10⁻⁹ torr. Data was then processed using Multipak™ software, and a Shirley background subtraction routine was applied [61].

A Field Emission Scanning Electron Microscope (FE-SEM) Hitachi S-4800, operating at 10 kV, equipped with an electron dispersive X-ray detector, was used for sample imaging and for Energy Dispersive X-Ray (EDX) spectra. A transmission electron microscope (TEM; JEOL 2000FX, JEOL USA, Inc., Peabody, Mass.) operating at 200 kV, equipped with small area electron diffraction (SAED) was used for crystallographic characterization. TEM sample preparation was done using standard procedure based on slicing, gluing, dimpling, and finally ion milling by Argon at 4 keV.

Atomic force microscopy (AFM) was performed at the AFM-SEM core of the Houston Methodist Hospital Research Institute and at the Medical School in Swansea University. The surface topography and the root-mean square roughness of the substrates was examined using the Bruker Multimode 8 with ScanAsyst, Santa Barbara, Calif. in Peak Force Tapping mode (scan rate: 1 Hz, sample/line: 256) with silicon probe (Bruker model RFESP-75, with a measured spring constant of 2.857 N/m). Roughness Rq was measured using the specific routine included in the Nanoscope Analysis software, v1.50, which calculates

$$Rq = \sqrt{\frac{\sum {Z}_{i}^{2}}{N}} ,$$

where, N is the number of points in the considered area and Zi is the vertical displacement of each point i from the average data plane. Three separate areas (of 25 μm² each) were collected for each surface type and Rq was calculated on subareas of 500×500 nm, with a total of about 60 Rq values for each surface type (either Si or Ti). Histograms of roughness values were plotted in Matlab 2019 (Mathworks, Natick, Mass.). The AFM NanoScope software's bearing and particle analysis functions were used to determine the thickness of the adsorbed protein. In the bearing analysis, it is assumed that all adsorbed proteins are detectable as topographic features [62, 63]. The analysis provides a plot of the histogram of surface height (depth) over a sample. Surface area and height above a threshold depth were then calculated using the particle analysis tools. The threshold depth was determined using the surface height distributions of dry Si and TiB₂ control samples, without adsorbed protein.

Surface wettability was determined via contact angle measurements. Samples were cleaned by ultrasonication in ethanol and DI water and dried using N2 air gun. Contact angles were measured using the sessile drop (5 μl) method with the Matrix 8300 Eletrapette Programmable Piper instrument. Images of contact angle were recorded with a digital camera, and measurements were using three each of Si and TiB₂ substrates.

Nanoindentation was performed on the substrates for a depth of 60 nm using MTS nanoindenter XP (Keysight Technologies, UK) and the Oliver and Pharr's analysis technique [64] to determine elastic modulus and hardness. The instrument was operated in the continuous stiffness mode and the indentations were made using a tip of 20 nm diameter. Ten indents were taken and averaged.

Surface charge assessment of Si (p-type and n-type) and TiB₂ substrates in DI water and culture media was performed via open circuit potential (OCP) measurements. Substrates were mounted on a customized sample holder and measurements were made using Autolab PGSTAT12 Potentiostat with NOVA software (Metrohm, Riverview, Fla.). The reference electrode was silver/silver chloride calibrated versus normal hydrogen electrode (NHE) at −0.265 V. Measurements provided steady state potential with no current flowing in the system. To enable appropriate scanning time and stable OCP measurements, the limit for potential change during the measurements was dE/dt<10⁻⁶ V/s. Measurements in DI water were used to calibrate the baseline reference values for interpretation of the corresponding readings in culture media.

Cell Culture. Human Umbilical Vascular Endothelial Cells (HUVECs; CC-2519, Lonza, Allendale, N.J.) were cultured in conventional tissue culture plastic flasks (TCPS) coated with 0.2% gelatin in Medium 199 (11150067, ThermoFisher Scientific, Waltham, Mass.) supplemented with 20% of fetal bovine serum (FBS, F4135), 1% antibiotic antimycotic solution (A5955), 1% Heparin (H3393), 1% HEPES (H0887) and 1% Endothelial Cell Growth Supplement (ECGS, E2759). Cells were grown to confluency at 37° C. with 5% CO₂ in a humidified controlled environment, and subsequently split for experimentation and further passaging. Human adult bone marrow MSCs characterized using flow cytometry for negative for CD14, CD34, CD45 expression, and positive CD73, CD90 and CD105 expression were obtained from Methodist Hospital under an IRB approved MTA. MSCs were cultured in TCPS in DMEM (MEM, M8042) supplemented with 10% FBS at 37° C. and 5% CO₂ environment. Unless stated otherwise, all reagents were purchased from Sigma-Aldrich Corp., MO, USA.

Cell culture of ovarian cancer cell lines: SKOV3 and OVCAR3 cell lines were cultured in Cancer Media (CM) (20% RPMI 1640 with glutamine (02-0205 VWR Life Science), 1% antibiotics (Sigma-Aldrich A5955), and 0.1% insulin (ABM TM053)). For cell culture on substrates CM was supplemented with 10 ng/mL human FGF2 (Sigma-Aldrich F0291)+1% heparin (Sigma-Aldrich H3393). Cells were cultured in a humidified incubator at 37° C. with 5% CO₂.

Cells from passage 1 to 9 times at a cell density of 300-600/mm² were used for seeding patterned substrates, which were cleaned and sterilized in a standard process using acetone, methanol, IPA, DI water and 70% ethanol. There was no biochemical or chemical functionalization of the substrates prior to cell culture.

Optical Imaging and Image Analysis. For immunofluorescence staining, cells were fixed with fresh 4% paraformaldehyde for 15 min at room temperature, and permeabilized with 0.1% Triton X-100. Rhodamine-conjugated phalloidin was used to stain the actin filaments and vinculin staining was used for focal adhesions. The following cell type specific biomarkers were analyzed: cd-31 (platelet endothelial cell adhesion molecule 1), cd-105, n-cadherin, and 4′,6-diamidino-2-phenylindole (DAPI) was used to stain DNA in the nuclei.

Morphological and viability assessment of the HUVECs grown on the patterned substrates was performed via Episcopic stereomicroscopy (reflected light) of the unstained cells on substrates using the SZX-10 (Olympus America, NY) and fluorescence confocal microscopy (FluoView-1000, Olympus America, NY), respectively. Fluorescence microscopy included used of acridine orange (AO; 3 μg/ml in phosphate buffered saline) to quantify cell viability [65], and immunofluorescence imaging to assess functional phenotype with cell specific monoclonal antibodies [66]. Two methods were used to quantify cell growth. (1) Percentage of viable cells incorporating AO using epifluorescence confocal microscopy. Three to five images of circle patterns per substrate at each time point were analyzed using ImageJ to determine the percentage of viable cells covering the circular TiB₂ patterns. Note that one to three substrates were randomly picked at each time point without replacement. The percentage area of the pattern covered with viable cells at different days following seeding was determined. At least four single circle patterns from each substrate at each time point were analyzed from two repeats, and the average value determined. (2) Percentage of morphologically intact attached cells (unstained/label-free) using stereomicroscopy. For cell counts, images of the substrate prior to seeding were used to generate image masks of the TiB₂ pattern areas on the background Si. Images captured at time points after seeding were manually aligned to the image masks, contrast enhanced, and cells were delineated using edge detection. Cell counts were determined per unit area by using perimeter lengths of 200 μm and 100 μm for MSC and HUVEC, respectively. A minimum of three repeats with 6-24 images per time-point for each repeat were analyzed. Cell alignment was assessed by quantitatively measuring the orientation of attached cells along the patterns using stereomicroscopy. The length, width, and axis of alignment of single cells were identified by manually outlining cells from stereomicroscope images. A length-to-width ratio of 1.0 indicates round or cuboidal shape, whereas ratios greater than 1.0 illustrate “longer-than-wide” or elongated cellular shapes. Cell shape (length-to-width ratio) measurements were performed on patterned substrates from three repeats. For each repeat, one to three substrates that had line patterns of width 5, 10, 20 30, 40, 45, 50 and 150 μm were analyzed. The substrates were imaged at different time points after seeding for a period of two weeks. At each time point, cells ranging in number from 3-15 were analyzed for the line patterns of varying width.

Cell orientation analysis was performed on five substrates that had line patterns of width 5, 10, 20 30, 40, 45, 50 and 150 μm. The angle between the cell axis and the pattern axis was used as a measure of cell alignment, with an angle of 0°/180° indicating perfect alignment and an angle of 90° indicating perpendicular alignment [67]. Approximately 3-20 cells per line per substrate were analyzed.

Viability analysis of the MSCs in aggregates on the substrates was performed using double fluorescent staining with acridine orange/propidium iodide (AO/PI) [68], in conjunction with laser scanning confocal microscopy. AO stains the DNA of live cells, and PI is membrane impermeable dye that only enters dead cells with damaged plasma membranes. Substrates with MSC aggregates were stained with a mixture of AO/PI solution (3 μg/ml AO and 10 μg/ml PI in phosphate buffered saline) for 20 min at room temperature and imaged using confocal microscopy.

Size of aggregates was measured from z-stacks of confocal images of the DAPI stained nuclei. The diameters of the aggregates were manually outlined and the thickness (height) was computed based on the number of z-sections with DAPI stained nuclei.

Image analysis of both immunofluorescence and stereomicroscope images was performed using ImageJ interactively and/or customized scripts [69].

RNA-Sequencing (RNA-seq) Library Preparation, Sequencing and Transcriptome Analysis. RNA was extracted using MiRNeasy Mini Kit with on-column RNase-Free DNase, digestion following manufacturer's instructions (Qiagen, Germantown, Md.). Extracted RNA samples underwent quality control assessment using the RNA tape on Tapestation 4200 (Agilent, Santa Clara, Calif.) and were quantified with Qubit Fluorometer (Thermo Fisher Scientific, Waltham, Mass.). Samples with a RIN score >8.80 were further processed for sequencing. The RNA libraries were prepared and sequenced at the University of Houston Seq-N-Edit Core per standard protocols. RNA libraries were prepared with QIAseq Stranded Total RNA library Kit using 100 ng input RNA (Qiagen, Germantown, Md.). The size selection for libraries was performed using SPRIselect beads (Beckman Coulter Inc., Brea, Calif.) and purity of the libraries was analyzed using the DNA 1000 tape Tapestation 4200. The prepared libraries were pooled and sequenced using NextSeq 500 (Illumina Inc., San Diego, Calif.); generating ˜20 million 2×76 bp paired-end reads per samples.

Raw fastq files were analyzed using FastQC [70], a quality-control tool for high throughout sequencing data that provides a set of parameters (e.g., sequence quality or presence of duplicated reads) allowing a quick assessment of data feasibility for further analysis. RNA-seq fastq files were then processed with CLC Genomics Workbench v12 (Qiagen, Germantown, Md.). The adaptors were trimmed, and reads were mapped to hg38 human reference genome. Read alignment was represented as integer counts by using parameters of mismatch cost 2, insertion cost 3, deletion cost 3, length fraction 0.8, similarity fraction 0.8, max of 10 hits for a read. Integer read counts were normalized by Trimmed Means of M-values (TMM) algorithm, finally generating a gene count matrix of sequencing reads. The gene count matrix was used to filter transcripts that were present or absent in each of the experimental conditions; the lists of genes were compared using Venn diagrams [71]. Additionally, after normalization of read counts, the inventors performed differential gene expression using the EdgeR package [72], which uses a generalized linear model linked to the negative binomial distribution to identify significance. The significance level of FDR adjusted p-value of 0.05 and a log₂ fold change greater than or equal to 2 was used to identify differentially expressed genes. Raw and processed RNA-seq data is deposited in the NCBI GEO Dataset (accession number GSE135824).

The platform PANTHER was used to perform functional classification analyses, statistical overrepresentation tests and statistical enrichment tests [73, 74]. The PANTHER GO terms selected to undertake functional classification analyses were “biological process” and “molecular function”; the results are displayed as pie charts. All major GO terms were tested for over-representation (Binomial test) comparing the lists of genes expressed in opposing experimental conditions (e.g., for cells cultured on the substrate (HUVEC TiB₂) versus control cells in conventional tissue culture flasks (HUVEC Plastic)). Similarly, all major GO terms were tested for enrichment using the lists of differentially expressed genes; results display the different distribution of significantly enriched clusters of genes compared to the overall expression tendency.

### Example 2—Results

Surface characterization of micropatterned substrates. Using circular micropatterns to control cellular morphology has been shown to be useful in varying the stemness of MSCs, for maintaining their multipotency [47] as well as inducing MSC spheroid formation [46]. Similarly, line micropatterns have been extensively used to achieve micro-vessel formation and evaluate vascular remodeling processes using HUVECs [45, 75, 76].

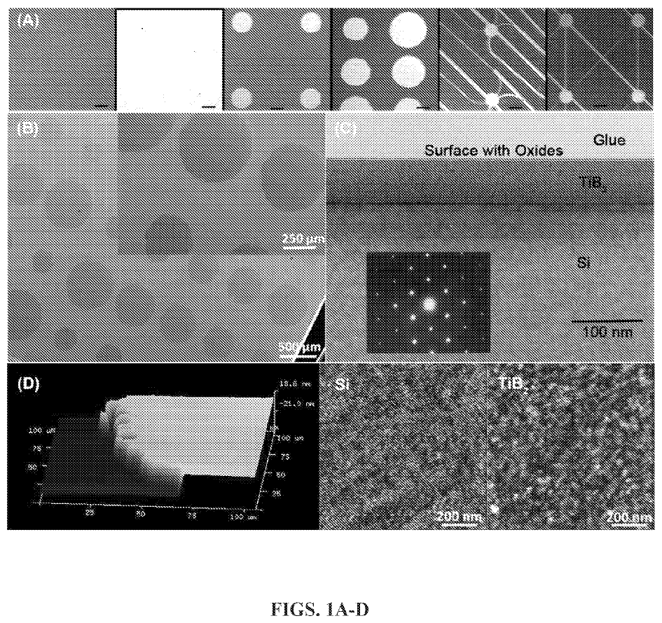

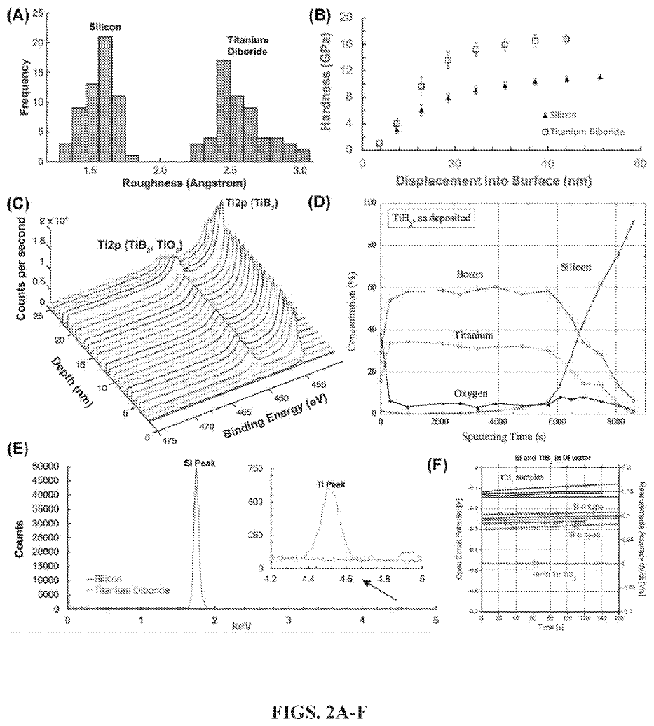

Stereomicroscopy images of unpatterned Si and TiB₂ and arrays of Si—TiB₂ circles with diameters ranging from 100 to 600 μm as well as variable lines (widths between 5 to 50 μm and lengths up to 500 μm) are shown in FIG. 1A (Si (darker) and TiB₂ (brighter)). SEM imaging also highlighted the background and patterned area on the substrate, with Si (lighter) and TiB₂ (darker) as seen in FIG. 1B. Furthermore, cross-sectional TEM analyses indicated that the TiB₂ layers were 30-40 nm thick and uniform with thin (— 2 nm) surface oxide layers (FIG. 1C). Selected area electron diffraction patterns (SAED) revealed that the TiB₂ films demonstrated an amorphous crystallographic structure (Single-crystal (spot) diffraction was from <110> zone axis orientation; see inset in FIG. 1C). Complementary measurements (data not shown) of annealed borides showed a hexagonal structure with lattice parameters, a=3.038 Å, c=3.27 Å (d100=2.63 Å d101=2.05 Å and d002=1.64 Å) for the 100, 101, and 002 lattice fringe diffraction lines) corresponding to TiB₂ [77, 78]. Anologus test results indicating stoichiometric compositions were obtained for ZrB₂ and HfB₂, respectively [177]. Crystalline oxides such as TiO₂ anatase and rutile were not identified at the surface or in the bulk of the films. AFM analysis enabled Si—TiB₂ 3 D surface representation, confirming the recorded thickness of 40 nm and highlighting similar surface topography of the Si and TiB₂ areas (FIG. 1D). Quantification of the surface roughness (Rq) confirmed small differences, with Si background exhibiting a lower mean Rq of 1.6±0.1 Å, whereas TiB₂ micropatterns exhibited a slightly higher mean Rq of 2.6±0.2 Å (see FIG. 2A). As seen in FIG. 2B, nanoindentation measurements recorded TiB₂ hardness at 14 GPa and Young's Modulus at 200 GPa compared to values of 10 GPa and 150 GPa, for Si. These results for TiB₂ show smaller values than those published for thick film boride [79], but matched values for thinner films as published by Zyganitidis (2009) [80]. X-ray photoelectron spectroscopy (XPS) was used to determine the molecular composition of the deposited diborides layers, with depth profiling conducted using Ar in situ sputtering [81, 82]. XPS indicated the presence of thin B- and metal-oxides and mixed oxides at the top surface layer for all analysed diborides (shown in FIG. 2C only for TiB₂ with only TiO₂ peak marked) and pure stoichiometric diborides (shown for TiB₂) composition in the bulk of the films, which was also confirmed by depth profiling (FIG. 2D) Analogous XPS results were obtained for ZrB₂ and HfB₂ showing stoichiometric compositions of these diborides in the bulk and B₂O₃ and mixed (respective) metal oxides at the surface only (plots not shown). C-1s was observed as a typical contaminant, mainly adsorbed at the surface and surface nitrogen levels were also low, i.e., below 0.46% atomic, with no formation of nitrides such as TiN or BN. This may be due, in part, to the strong susceptibility to oxidation, similar to Ti, which reacts with oxygen instantaneously and forms very thin oxides (such as TiO and Ti₃O₂) in air even at RT [83]. In line with previous reports on oxidation, the Ti⁴⁺ state was more predominant while TiO₂ was frequently formed at elevated annealing temperatures [84]. Such oxide layers were stable and passivated the surface due to limited oxygen diffusion through the oxide [85-87].

Further chemical characterization was conducted in the form of energy-dispersive X-ray spectroscopy (EDX), showing a lower Si peak on the TiB₂ layers (FIG. 2E), while a weak Ti peak was seen on only the TiB₂ patterned areas. As shown in FIG. 2F, larger negative surface charges were recorded on the silicon surface for both n- and p-type than on TiB₂ surface, as measured by open circuit potential (OCP) obtained in DI water.

OCP values for Si were (“p-type” −0.24V to −0.27V, “n-type” −0.22V to −0.23V), whereas those for boride, TiB₂ were −0.082 V to −0.14V). These values concur with published point of zero charge (PZC) or isoelectric point (IEP) values reported for Si and SiO₂ [88-91], and Ti, and TiO₂ [92].

This is expected because the work function of TiB₂ is larger than that of low doped Si and values of IEP for all transition metals oxides from group 4^{th} are significantly larger than that for silicon dioxide [186]. Electron work function values for the 4^{th} group transition metal borides are large (4.71-4.96 eV TiB₂, 4.41-4.85 eV ZrB₂, 4.71-4.86 eV HfB₂) [175] indicating chemical stability and surface charging. Silicon low doped n- and p-type have similar values that are not associated with Fermi level differences defined by the conductivity type and doping concentration levels. Potential of zero charge (PZC) is larger for pure Ti than for Si, thus in solutions at the same pH, Ti exhibits fewer negative charges at the surface. Studies have shown that for Ti alloys, boron shifts the surface potential to more acidic thereby making the surface more negatively charged [93]. However, adding a thin coating of TiB₂ results in less negative potentials than those seen for Ti indicating smaller negative charges [94, 95], as is seen with the inventors' substrates wherein the boron in TiB₂ at the surface is in the form of thin oxide and in aqueous solution also as boric acid. Similarly, more positive surface charges can be expected for ZrB₂ compared with silicon based on its isoelectric point at pH=6.7 [187]. Behavior of the inventors' Si and TiB₂ substrates show small changes of OCP (boron effect) in time, which may be indicative of oxide buildup [96]. Clean TiB₂ patterns (and other diborides for the 4^{th} group) exhibited hydrophilic surface with contact angles ranging from 16-20°, while the background SiO₂/Si substrate was less hydrophilic with a contact angle at 45°.

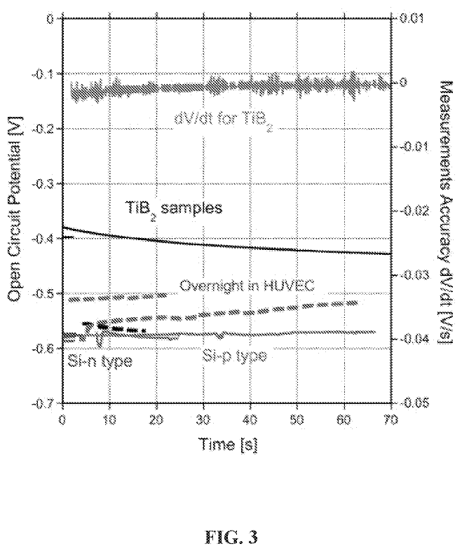

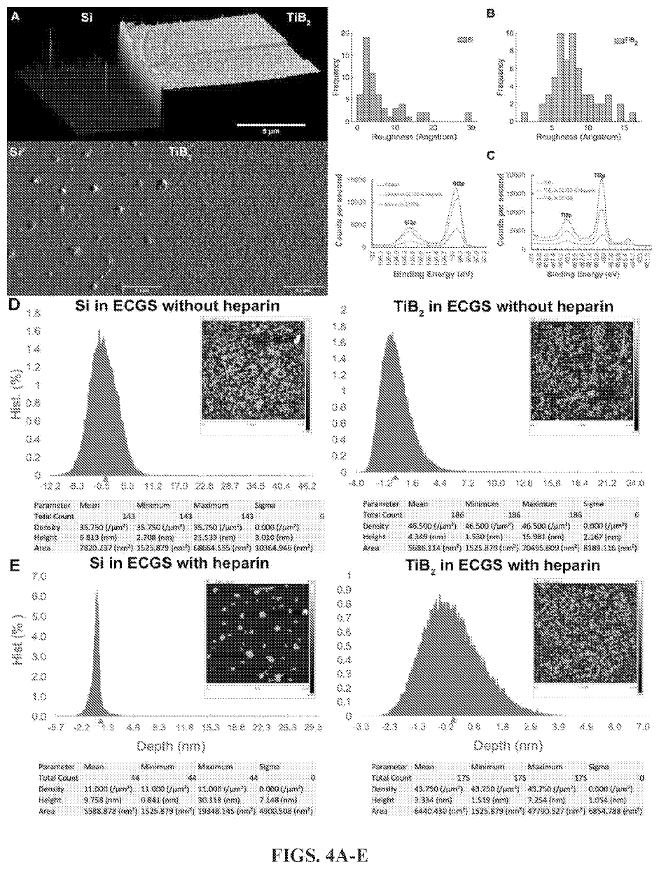

Surface characterization of micropatterned substrates exposed to supplemented culture media. OCP analysis of Si and TiB₂ substrates in culture media supplemented with ECGS and heparin showed significant increase in negative charge for both materials, with increased difference between the two at the onset of measurements (FIG. 3).

The increasing negative charges are unrelated to any etching/corrosion in time as evident from etch-free surface quality. OCP values for TiB₂ dropped to −0.4V and that for Si dropped to −0.6V upon immersion into supplemented culture media. It should be noted that for few measurements the OCP remained unchanged and further measurements were terminated as determined by the set potential change limit of 10⁻⁶ V/s. Adsorption on the surface by culture media components may be responsible for the observed time dependence of OCP [97]. Adsorption of media component layers at the surface of each material become independent of the original substrate charges in prolonged immersion, as seen in Si and TiB₂ samples tested after overnight storage in supplemented culture media. These results indicate that electrostatic effects are mostly important at the beginning of the culture process during substrate seeding when proteins from the media adsorb at the surface of the substrates.

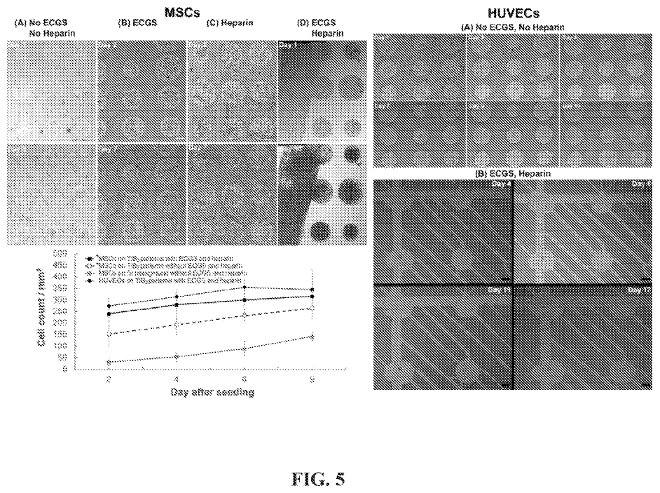

AFM and XPS were carried out on clean substrates as well as those incubated overnight in ECGS, with and without heparin, to assess protein adsorption on the micropatterned TiB₂ substrates. On both unpatterned TiB₂ layer and Si substrates, incubated with 50 μg/mL ECGS, a clear layer of protein deposition was observed; however, a reduction in protein deposition was observed on background Si in the presence of 1% heparin, suggesting that heparin mediates differential protein adsorption on TiB₂ (FIGS. 4A-E). In the presence of ECGS and heparin, while the topography of the TiB₂ area is relatively uniform, the Si area shows randomly scattered globular deposits (also evident as scattered peaks in the 3D surface map in FIG. 4A). These observations are confirmed with Rq analysis shown in FIG. 4B, where the Si background exhibits a wide range of Rq values from 0 to 30 Å with a mean of 6.2±6.4 Å. TiB₂ micropatterns exhibit a higher, more uniformly distributed Rq range between 3.6 and 10.4 Å, with a mean of 8.0±2.9 Å. Furthermore, XPS results confirmed the AFM observations, showing a percentage reduction in elemental concentration for both Si and TiB₂ and increased organic matrix elements, when incubated overnight in 50 μg/ml ECGS as seen in (FIG. 4C) and Table 1. The AFM images and histograms of the bearing analysis for Si and TiB₂ when exposed to ECGS with and without heparin are shown in FIGS. 4D-E. In the inset AFM images protein features are highlighted in cyan to distinguish them from background substrate features. Overall similar profile shapes for the surface height distribution were observed, but the distributions were shifted to different depths. As seen in FIG. 4D, the surface heights distribute in a relatively similar range across TiB₂ and Si in the absence of heparin, with mean values of 4.4±2.2 nm (range: 1.5-16.0 nm) and 6.8±3.0 nm (range: 2.7-21.5 nm), respectively. In the presence of ECGS and heparin (FIG. 4E), mean surface thickness on TiB₂ was 3.3±1.5 nm (range: 1.5-7.3 nm), while that for Si was and 9.8±7.1 nm (range: 0.8 nm-30.1 nm) indicating isolated clumped regions of proteins distributed over Si.

Table 1 presents the elemental composition of material adsorbed on the substrates in the presence of different media supplements as determined by XPS. Elemental concentrations of Si and Ti (and B) were used to assess the exposed surface of the Si and TiB₂ substrates, respectively. Additionally, the elemental concentration of C, N, and S, were used to assess the relative amount of adsorbed protein [98]. Adsorption of proteins from FBS or ECGS on both Si and TiB₂ was high, in that >50% total of C, N and S was present on the surface compared to lower amounts of Si (11-13% compared to 51.6% control) and Ti (1.4-2.8% compared to 13.8% control). Adsorption of heparin on the Si or TiB₂ substrates was minimal as surface concentration of Si (45.8%) was similar to that of control (51.6%). Similarly, surface concentration of Ti (15.2%) and B (15.8%) was comparable to control values of Ti (13.8%) and B (16.8%). As measured by open circuit measurements both Si and TiB₂ substrates exhibit negative surface charges and are likely to repel the adsorption of heparin which has the highest net negative charge density of all known biological molecules [99]. In the presence of heparin and ECGS, protein adsorption is seen on both Si and TiB₂ substrates, but relatively higher protein adsorption was seen on TiB₂ (the surface Ti content reduces to 6.6% compared to 13.8% control), compared to the background Si where protein adsorption was relatively less, with 38.4% of Si measured at the surface compared to 51.6% of control. Heparin is a negatively charged highly sulfated heparan glycosaminoglycan that binds with high affinity to a large number of proteins such as growth-factors containing positively charged, heparin-binding domains [100]. ECGS constitutes a cocktail of human growth factors containing heparin-binding domains, including basic fibroblast growth factor (FGF), insulin-like growth factor-1 (IGF-1), vascular endothelial growth factor (VEGF), and endothelial growth factor (EGF) [101, 102]. Thus, in the presence of heparin and ECGS, heparin-bound growth factors are adsorbed on the micropatterned substrate, with higher concentration on TiB₂. Si is more negatively charged compared to TiB₂, thereby repelling the heparin bound ECGS proteins and resulting in relatively lower level of protein adsorption. Notably, heparin does not significantly affect the deposition of proteins from FBS on Si or TiB₂. In the presence of heparin and FBS, most of the substrate is covered with protein as indicated by low surface concentrations (<2%) of Si and Ti, and high concentrations (>58%) of C, N and S. This is due to the reduced amounts of proteins with heparin binding domains in FBS when compared to ECGS [101-103]. Interestingly, in the presence of FBS, ECGS and heparin, protein adsorption is high on both Si and Ti, as indicated by low surface concentration of Si and Ti (<1%), and high concentrations (>56%) of C, N and S. However, it is likely that a larger fraction of heparin-bound ECGS proteins are on TiB₂, given that proteins from ECGS in the presence of heparin adsorbs less readily on Si with it more negative surface potential. These data demonstrate that in media supplemented with ECGS and heparin, there is higher protein adsorption on TiB₂ micropatterns relative to the Si background.

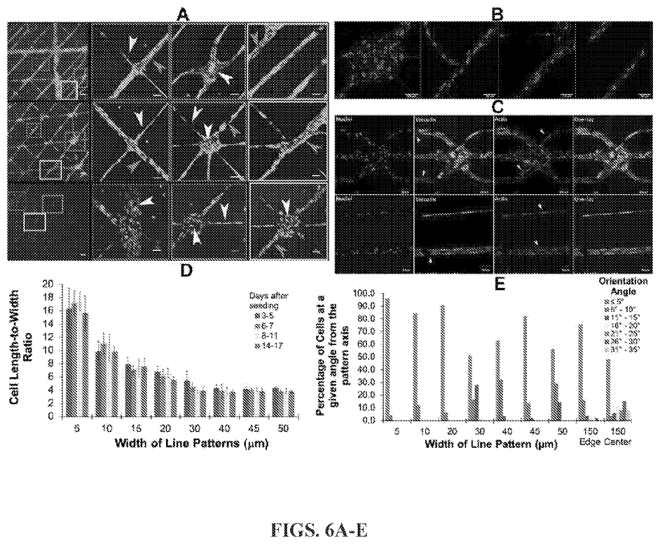

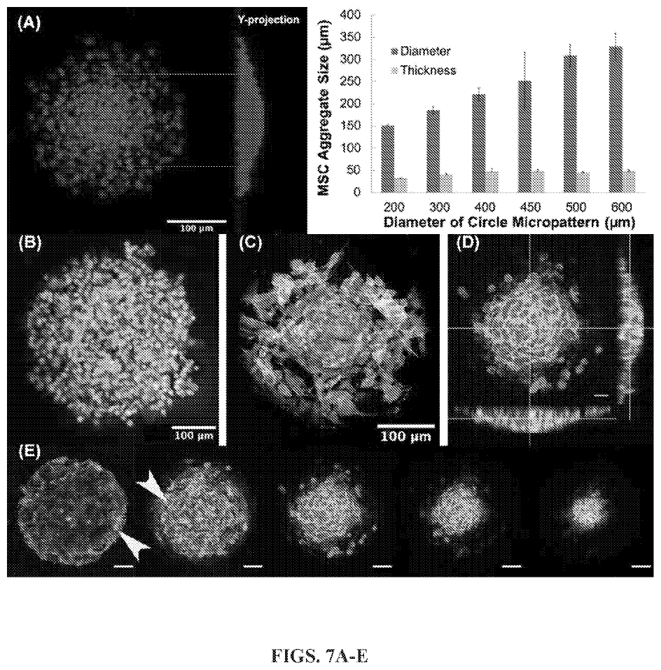

Cellular patterning of HUVECs and MSCs on the micropatterned substrates. The inventors assessed the ability of HUVECs and MSCs to adhere and grow on the TiB₂ micropatterned substrates over a period of 2-3 weeks by acquiring stereomicroscope images at intervals of 24-48 hours. Representative reflected light images from stereomicroscopy of HUVECs and MSCs cultured on the Si—TiB₂ substrates in (i) ECGS-free and heparin-free media, (ii) culture media supplemented with ECGS, (iii) culture media supplemented with heparin, and (iv) culture media supplemented with ECGS and heparin are presented in FIG. 5. As seen in FIG. 5 (top left panel), in the absence of both ECGS and heparin, some MSCs adherence is observed on both the Si background and the TiB₂ patterns. MSCs however prefer the TiB₂ patterns over the background Si, as is evident by the significantly larger number of cells adhering to the circle patterns (Supp. Table 2). In the presence of ECGS only, the cells prefer the patterns initially but over time grow over onto the background Si area. Similarly, in the presence of heparin only, although the cells initially show preferential growth on TiB₂ patterns, they later grow into a confluent monolayer over the substrate. Notably, following over a week in culture, MSCs on the circular TiB₂ patterns spontaneously group and cluster to form 3D aggregates on circle patterns. Interestingly, in media supplemented with ECGS and heparin, cell adherence and growth are highly specific for TiB₂ micropatterns, with no cell adherence or growth observed on the Si background throughout the culture week (tested over a three-week period, see also SFIGS. 1A-B). MSC aggregation occurs as early as day 5 followed by 3D aggregate formation by the end of the week.

As observed with MSCs, in the presence of ECGS and heparin, HUVECs too show highly specific growth on the TiB₂ patterns with no growth on the Si background. However, unlike MSCs which are able to grow in media without supplements, HUVECs fail to adhere and thus they are unable to show any growth on the substrates in the absence of ECGS and/or heparin (see FIG. 5, right panel). This is expected behavior because growth factors and heparin have been demonstrated to be critical for any in vitro HUVEC culture proliferation [104-106]. Cell proliferation was quantified using image analysis macros to segment cells grown on Si background versus patterns for different media supplements (see SFIGS. 2A-G). Two to four images per day were analyzed for each repeat. Four repeats were analyzed for MSCs grown in supplement-free media, six repeats were analyzed for MSCs grown in supplemented media, and 3 repeats were analyzed for HUVECs grown in supplemented media. Cell counts increase over a period of 9 days, with higher number MSCs seen on the TiB₂ patterns compared to the Si background for supplement-free media (Supp. Table 2). Cell counts are significantly different over time and across the supplemented and supplement-free samples (Supp. Tables 1-3), with the exception of days over a week, when aggregation occurs and the total number of cells counted are underestimated, because of the inability to get 3D data from 2D images. HUVECs also approach steady state growth over a period of one week (Supp. Table 1 and FIG. 5). HUVECs are unable to grow on the Si background in the absence of ECGS and heparin (FIG. 5, right panel), hence the cell counts are presented for supplemented media only. These results unequivocally demonstrate that TiB₂ pattern specific growth is achieved only in the presence of ECGS and heparin. The same growth selectivity was obtained for the same patterns created in ZrB₂ and HfB₂ layers deposited by e-beam evaporation on the silicon substrates.

Importantly, the shape of these borides' micropatterns influences cell growth patterns. Previous work has shown that circle patterns specifically promote cell aggregation [57, 107]. The inventors observed similar growth patterns, in that, circular patterns promoted 3D MSC aggregate formation (FIG. 5; left panel), and SFIGS. 1A-B (red arrows), whereas MSC grew in undefined layers on the contiguous un-patterned TiB₂ when not restricted by pattern geometry (FIG. 5; left panel), and SFIG. 1B (blue arrows). MSCs were also cultured in supplemented media on substrates with circle and line patterns (see SFIGS. 1AB and SFIG. 3). In addition to aggregation of MSCs on the circle patterns, the inventors observed that the proximity of lines to the circle patterns resulted in cells stretching across the lines and circles (see white arrows). Notably, MSCs showed contact guidance, aligning along the line patterns (of 5 μm width). As seen in FIG. 5 (right panel), HUVECs were also cultured on both circles only and the circle and line patterns.

The longest duration for cell growth observed in this study was three-weeks for both MSCs and HUVECs. Images showing MSC growth progression at 24 to 48 hour intervals for a period of 2-3 weeks on substrates with different patterns are shown in SFIGS. 1A-B. In vivo MSCs are migratory and are known to move from the bone marrow, migrate through tissue, and home in on an injury site. MSC homing can occur via chemical stimulus or under durotactic cues that results in directed migration of the cells in response to stiffness gradients. Recently, Vincent et al., 2013, demonstrated initial attachment and spreading of MSCs independent of gradient strength or stiffness within hours of seeding, and consequently after 3 days, the cells migrated to stiffer portions of the substrates [108]. In this study, the inventors observed similar results with non-specific stiffness dependent preferential growth of MSCs on the micropatterns in the absence of ECGS and heparin, and specific growth and 3D aggregate formation on the patterns in the presence of ECGS and heparin.

Commercially available ECGS is largely used to support the expansion of endothelial cells, and most formulations constitute a cocktail of human growth factors including basic fibroblast growth factor (FGF), insulin-like growth factor-1 (IGF-1), vascular endothelial growth factor (VEGF), and endothelial growth factor (EGF) [101, 102]. Notably all these growth factors have a heparin binding site [109], and recently a growing number of physiological processes, such as cell adhesion, migration and proliferation, have been shown to be regulated by the action of heparin [100, 110-112]. In order to identify if MSC specificity and 3D aggregation was limited to a particular growth factor, the inventors supplemented media with 5 nM, 10 nM, or 20 nM concentrations of FGF, EGF or VEGF, and 1% heparin. Micropattern specific growth and MSC 3D aggregation were observed for each growth factor tested (data not shown). This suggests that specificity was not limited to a particular growth factor supplement or combination, rather any individual growth factor with a heparin binding domain can be used to achieve diboride pattern-specific growth and 3D aggregation.

HUVEC Viability, Phenotype and Morphological Characterization. The micropatterned substrates were found to be biocompatible for HUVEC growth, with selective growth patterns observed according to well-established effects of pattern alignment and contact guidance [37, 45, 113-23, 117]. Images of HUVECs stained with viability dye AO demonstrate preferential adherence and growth of the cells on the geometric micropatterned TiB₂ substrates with variably sized circles and lines (FIG. 6A; rows 1-3). Images in column 1 are at 4× magnification, with selected areas (colored boxes) shown at a higher 20× magnification (FIG. 6A, columns 2-4). HUVEC cells growing on the TiB₂ patterns were elongated, aligned along the long axis of the pattern (FIG. 6A, red arrows) and well spread with boundaries of the outermost cells following the edges of the patterns to which they had attached (FIG. 6A, white arrows).

Pattern size also had a demonstrable effect, with cells spreading and colonizing the entire area (FIG. 6A, white arrows) in wider patterns that were 5-10 larger than the cellular diameters. If the pattern diameter was controlled and only double that of the cells seeded, the cells, while remaining elongated, line up adjacent to each other to form cell-by-cell arrays (FIG. 6A, red arrows). Finally, single cells were seen elongated and aligned along the patterns when line widths of narrow diameters were patterned (FIG. 6A, yellow arrows). These results confer with several previous studies that have shown pattern size- and shape-dependent controlled growth and alignment of a variety of cell types including fibroblasts, endothelial, mesenchymal, neural, cardiac and muscle (reviewed in [116, 118]). This selective preference of HUVECs is the result of the differential adsorption of growth factors on the patterned areas in the presence of heparin, mediated by differences in material properties including hardness, hydrophilicity, and charge of the diboride layers when compared to the Si background. Moreover, the presence of green fluorescence indicates the intercalation of the AO dye with DNA, thereby confirming overall health and viability of the cultured cells. Furthermore, as shown in other studies [119], the adherence and spreading of the HUVECs was influenced by the shape and size of the patterns.

In order to assess the adoption of a functional HUVEC cell phenotype following substrate culture, immunofluorescence staining for the platelet endothelial cell adhesion molecule 1 (PECAM-1, or CD31), actin (cytoskeleton) and vinculin (focal adhesions) was undertaken. The cells were counterstained with DAPI to visualize all cell nuclei. HUVECs express CD31 (red) when cultured on TiB₂ micropatterned substrates (FIG. 6B). On the circular pattern (FIG. 6B, column 1), the cells exhibited a “cobblestone” morphology with CD31 staining visible at the cell membrane surfaces that constitute the intercellular boundaries of the confluent monolayer. On the line patterns (FIG. 6B, column 2-4) the cells aligned along the directional axis of the patterns. These findings provide evidence that the HUVECs retain their characteristic phenotype by continued expression of PECAM when cultured on micropatterned surfaces while aligning to the geometric patterns.

HUVEC formed distinct attachment to the TiB₂ patterned substrate via the formation of cell adhesion complexes such as focal adhesions (vinculin) and cytoskeletal (F-actin fibers) rearrangements (FIG. 6C). Actin stress fibers aligned along the longitudinal axis of the TiB₂ line patterns and along the periphery of the circle patterns (see white arrows). For the narrow lines, actin filaments were clearly observed in perfect alignment with the pattern margins, illustrating cell-material interaction via “contact guidance” [120]. Since the patterns were not activated by biochemical means, i.e., they were not functionalized by any of the adhesion promoters such as fibrinogen or other proteins, these results are consistent with past work showing substrate stiffness can affect cytoskeleton orientation and formation of focal adhesions [121].

HUVEC morphology (shape; length-to-width ratio) and orientation (alignment) was also assessed in response to the circle and line patterns using stereomicroscopy images. The average value of the mean of the length-to-width ratio of cells is plotted against the different widths of the patterned lines in FIG. 6D. There is a significant difference in the elongation factor of cells when comparing cells on narrow width ≤20 μm to cells on the wider lines, with most elongated cells observed on the 5 μm lines. These results concur with those of Lei et al. [45], who showed restricted spread of endothelial cells on narrower patterns. As the width of the lines is increased, cell spreading is unrestricted along the width and this leads to an approximately 4× reduction in the elongation factor. A plot of the percentage of cells on the patterned lines of varying widths; aligning with the pattern axes at varying angles ranging from 5°-35° is also shown in FIG. 6E. Nearly perfect alignment (angles ≤5°) of cells to the pattern can be seen for patterns of line widths ≤20 μm for greater than 80% of the cells. This result is substantiated by the fact that the width of attached HUVECs can range in size from 5 to 20 μm [75], which would limit occupancy to no more than 1-2 cells on lines of width ≤20 μm. However, in line with published studies, increasing line widths above 20 μm enables several cells to grow along random directions unrestricted by the pattern margins and consequently result in larger orientation angles [45]. As seen in FIG. 6E very strong alignment of cells occur at the pattern edges compared to central parts of the line patterns. The 150 μm (edge) had ˜75% cells aligned at angles in the 0° to 5° range, ˜15% cells in the 5° to 10° range and about 10% cells in the 10° to 30° range. The central regions had ˜48% cells in the 0° to 5° range and the remaining 52% in the 10° to 35° range.

Cell growth and viability on the patterned substrates was further corroborated by determining the total area of viable cells covering the patterned surfaces. Significant growth was observed from day 4 until day 7 after seeding (p-value of 0.0002<0.05), following which a reduction in the number of cells is observed on day 11 (p value of 0.004<0.05), and subsequent maintenance of cell growth at a steady state (days 11-13; p>0.05) (SFIG. 4).

The size of MSC 3D aggregates on the circular micropatterns was determined from confocal stacks of the DAPI stained nuclei. As can be seen in FIG. 7A, the spheroid diameter was dependent on pattern diameter, with larger spheroids forming on larger circle patterns, whereas the thickness of the spheroids was relatively uniform ranging in size from 45-50 μm. FIG. 7B presents a maximum intensity projection of an MSC 3D aggregate stained with AO/PI. Cell viability within 3D aggregates, was found to be in the range of 64.3-89.0% with a mean value of 75.8±10%.

Also, phenotype (CD105, FIG. 7C), cell-cell interaction (N-cadherin, FIGS. 7D-E), and structural morphology (F-actin) assessment validated biomarker sustainability of MSCs cultured on the substrates. CD105 was present in the cells over a two-week culture period, with higher levels of distribution was observed in cells along the edges then those within the aggregates. FIG. 7D presents a maximum intensity orthogonal projection of an MSC 3D aggregate stained for F-actin (green), nucleus (DAPI, blue), and N-cadherin (red), whereas FIG. 7E presents individual z-slices through the aggregate.

As expected, clustering of N-cadherin is observed within 3D aggregates vs homogenous staining in cells at the pattern boundary. Also, notable, is the characteristic spindle-shaped stretching and anchoring of the F-actin cytoskeleton (green) in the individual confocal z-slices along the periphery of the circle patterns (see column 1 in FIG. 7E), versus the rounded clumping of the cytoskeleton in the interior cells seen with the 3D aggregate (FIG. 7E, columns 4-5).

Total RNA-sequencing Transcriptome Analysis. To identify gene expression patterns that were specific for HUVECs and aggregated MSCs on the TiB₂ micropatterned substrates, the inventors performed transcriptome profiling using an ultra-deep unbiased RNA sequencing (RNA-Seq) approach. MSCs and HUVECs cultured in monolayers in conventional plastic tissue culture flasks were used as controls. In the presence of heparin and ECGS, both MSCs and HUVECs fail to adhere to the background silicon as shown in FIG. 5 and SFIGS. 1A-B. Thus, the inventors were able to selectively isolate cells grown on micropatterns for RNA analysis.

Quality control analyses were performed on raw Illumina reads using FastQC in order to monitor the quality of the data [70]. QC metrics such as sequence quality, presence of adaptors or duplication levels, amongst other parameters, displayed high homology between samples and were deemed acceptable for pursuing the analysis of RNA-seq data.

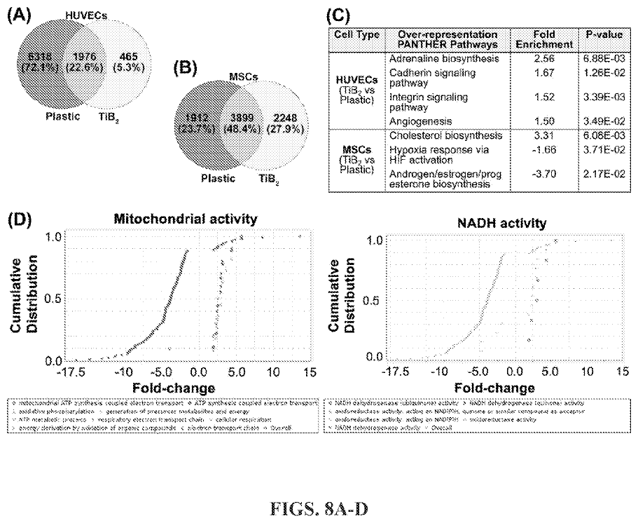

The sequencing analysis of HUVEC cells grown in normal tissue culture flasks identified 8294 different transcripts, whereas 2441 were identified in HUVEC cells grown on the TiB₂ substrates (FIG. 8A). To test the biocompatibility of the substrates at transcript level, the lists of identified genes were collated against the evolutionary relationship platform PANTHER. PANTHER utilizes a binomial test to associate a gene list provided by a user to a reference list (all genes in PANTHER database for the selected organism) for defined PANTHER GO terms [73, 74].

The inventors mapped the lists of transcripts that were expressed in HUVEC cells for major Go terms of “biological process” and “molecular function” (SFIGS. 5A and 5C and SFIGS. 6A-B), showing that despite disparities in the number of expressed genes, both cell populations displayed stable biological and molecular functions. The disparity in the number of genes may be the effect of microenvironmental factors. Studies have reported the dependence of in vitro culture of endothelial cells on microenvironmental factors such as the presence of a coated surface, availability of growth factors in the medium, and seeding density [122, 123]. Heng et al., 2011 have also demonstrated that seeding density has a profound effect on the proliferation and gene expression profile of HUVECs seeded on different biomaterial surfaces [122]. Based on the PANTHER analysis (SFIGS. 4-6), HUVECs cultured on the substrates exhibit a biological and molecular profile similar to those cultured on plastic.

In the case of MSCs, 5811 transcripts were identified when grown on plastic, and 6147 transcripts when grown on micropatterned TiB₂ substrates (FIG. 8B). PANTHER analyses corroborated the biocompatibility between surfaces (SFIGS. 4B and 4D). To gain insight into the pathways that are specifically enriched upon growth on micropatterned TiB₂ substrates, the lists of transcripts were evaluated for overrepresentation tests using PANTHER (GO term “PANTHER Pathways”). Enriched pathways identified in HUVECs and MCSs grown in TiB₂ are depicted in FIG. 8C and include processes such as ‘angiogenesis’ (HUVECs) and ‘cholesterol biosynthesis’ (MCSs). Interestingly, the ‘cholesterol biosynthesis’ pathway in MCSs is also significantly enriched in the set of unique transcripts expressed in the TiB₂ sample (2248, FIG. 8B) when compared with the unique transcripts expressed in plastic flasks (1912, FIG. 8B). This result suggests an increased cholesterol biosynthesis activity when MCSs grow on TiB₂ micropatterned substrates as opposed to typical culture flasks.

Results of differential gene expression analysis between HUVECs grown in plastic and TiB₂ substrates indicated that of all the genes detected, 109 significantly changed expression levels [Fold-change>±1.5, false discovery rate (FDR)<0.05]. Out of the 109 transcripts, 95 were downregulated and 14 were upregulated (Supp. Table 4). When evaluating differential gene expression for MSCs, only 22 genes significantly changed expression (Fold-change>1.5; FDR<0.05), of which 7 genes were downregulated and 15 were upregulated (Supp. Table 5). To gain further insight into which processes or pathways are significantly enriched within the list of differentially expressed transcripts, the inventors performed PANTHER enrichment tests. The list of differentially expressed genes in the case of MSCs (26) was too small to generate significant results; however, PANTHER was able to identify a number of enriched processes in HUVECs grown on TiB₂ substrates using the GO terms “biological process” and “molecular function” (FIG. 8D). Thus, processes related with mitochondrial ATP synthesis and NADH activity were significantly up-regulated in HUVEC cells on TiB₂ compared with the plastic control (FIG. 8D). A substantial number of mitochondrial genes were also up-regulated in MSC cells (Supp. Table 5), strongly suggesting a link between increased metabolic activities and cellular growth on the micropatterned TiB₂ substrate.

TABLE 1
Elemental Composition of Material Adsorbed on the Substrates in the
Presence of Different Media Supplements as Determined by XPS Analysis
Element content (at. %) on Si and TiB₂ substrate in the presence of

| | Control | | | | | | | | ECGS + | | Fbs + | | FEB_ECGS + | |
|---|---|---|---|---|---|---|---|---|---|---|---|---|---|---|
| | (Dry) | | FBS | | ECGS | | Heparin | | Heparin | | Heparin | | Heparin | |
| Element | Si | TiB₂ | Si | TiB₂ | Si | TiB₂ | Si | TiB₂ | Si | TiB₂ | Si | TiB₂ | Si | TiB₂ |
| Si | 51.6 | | 11.1 | | 12.8 | | 45.8 | | 38.4 | | 1.9 | | 0.9 | |
| Ti | | 13.8 | | 2.8 | | 1.4 | | 15.2 | | 6.6 | | 0.2 | | 0.09 |
| B | | 16.4 | | 0.2 | | 2.5 | | 15.8 | | | | 0.5 | | |
| C | 6.9 | 18.4 | 49.1 | 52.1 | 51.8 | 56.0 | 11.9 | 13.7 | 23.3 | 37.5 | 58.3 | 61.9 | 56.3 | 54.0 |
| N | | 0.7 | 11.3 | 12.0 | 13.3 | 13.9 | 1.3 | 1.4 | 3.9 | 8.1 | 13.6 | 13.5 | 12.0 | 12.0 |
| O | 41.5 | 52.2 | 20.3 | 25.8 | 21.5 | 21.8 | 40.3 | 52.2 | 33.2 | 37.3 | 20.3 | 19.4 | 20.8 | 22.2 |
| Na | | | 3.8 | 3.1 | 0.4 | 3.4 | 0.5 | 1.5 | 1.3 | 0.7 | 4.1 | 3.3 | 3.7 | 4.1 |
| S | | | 0.1 | 0.5 | | 0.2 | | | | 0.4 | 0.4 | 0.5 | 1.1 | 1.2 |
| Cl | | | 4.2 | 3.7 | | 1.1 | | 0.1 | | 0.5 | 1.5 | 0.8 | 5.1 | 3.4 |

Chemical state of various elements: Si was Si ~70-80% and SiO₂ ~30-20%, Ti was TiO₂ ~90% and TiB₂ ~10%, B was Boride and/or Boron ~30% and B₂O₃ ~70%, C was CHn ~40-60% and C with N, O ~60-40%, N was Ammonium Salt, Organic Matrix ~97% and Nitride, Cyanides, Azide ~3%.

SUPP. TABLE 1

| | | | MSCs | MSCs |
|---|---|---|---|---|
| | HUVECs | MSCs | growing in | growing in |
| | growing in | growing in | supplement- | supplement- |
| | supplemented | supplemented | free media | free media |
| | media on TiB₂ | media on TiB₂ | on TiB₂ | on Si |
| | patterns | patterns | patterns | background |
| Day 02-04 | 0.089 | 0.290 | 0.280 | 0.100 |
| Day 02-06 | 0.033 | 0.037 | 0.031 | 0.016 |
| Day 02-09 | 0.252 | 0.125 | 0.006 | 0.000 |
| Day 04-06 | 0.096 | 0.483 | 0.164 | 0.097 |
| Day 04-09 | 0.575 | 0.455 | 0.019 | 0.000 |
| Day 06-09 | 0.862 | 0.700 | 0.102 | 0.027 |

Students t-Test (two-sided, with a P-value <0.05 considered significant) comparing cell counts over a period of 9 days for HUVECs and MSCs on the micropatterned substrates. Two to four images per day were analyzed for each repeat. Four repeats were analyzed for MSCs grown in supplement-free media, six repeats were analyzed for MSCs grown in supplemented media, and 3 repeats were analyzed for HUVECs grown in supplemented media.

| | SUPP. TABLE 2 |
|---|---|
| | MSCs in supplement |
| | free media |
| | Si vs. TiB₂ |
| Day 02 | 0.004 |
| Day 04 | 0.001 |
| Day 06 | 0.000 |
| Day 09 | 0.000 |

Students t-Test (two-sided, with a P-value <0.05 considered significant) comparing MSC counts on the micropatterned substrates over a period of 9 days in supplement-free media on Si vs. TiB₂. Two to four images per day were analyzed for each repeat. Four repeats were analyzed for MSCs grown in supplement-free media.

SUPP. TABLE 3

| | MSCs growing in | MSCs growing in |
|---|---|---|
| | supplemented media on | supplemented media on Si |
| | TiB₂ patterns vs. MSC | background vs. MSC |
| | growing in supplement- | growing in supplement- |
| | free media on TiB₂ patterns | free media on Si background |
| Day 02 | 0.037 | 0.000 |
| Day 04 | 0.048 | 0.000 |
| Day 06 | 0.004 | 0.000 |
| Day 09 | 0.324 | 0.007 |

Students t-Test (two-sided, with a P-value <0.05 considered significant) comparing MSC counts on the micropatterned substrates over a period of 9 days in supplemented vs. supplement-free media. Two to four images per day were analyzed for each repeat. Four repeats were analyzed for MSCs grown in supplement-free media, six repeats were analyzed for MSCs grown in supplemented media.

SUPP. TABLE 4

| | Fold | | | Fold | |
|---|---|---|---|---|---|
| SYMBOL | change | FDR | SYMBOL | change | FDR |
| VASH1 | 8.48 | 0.017 | MYH9 | <−10 | 0.002 |
| RPL35A | 5.72 | 0.033 | CPM | <−10 | 0.001 |
| MT-ND2 | 5.67 | 0.001 | CCDC85B | | |
| TAGLN2 | 4.77 | 0.032 | MPP5 | <−10 | 0.001 |
| MT-ATP8 | 4.42 | 0.001 | CLN5 | <−10 | 0.001 |
| MT-ND1 | 4.37 | 0.001 | NFE2L3 | <−10 | 0.001 |
| Mt-atp6 | 3.58 | 0.001 | FAM184B | <−10 | 0.014 |
| MT-ND4 | 3.16 | 0.001 | FLNA | <−10 | 0.001 |
| MT-ND4L | 2.99 | 0.001 | CD302 | −9.71 | 0.001 |
| MT-CO2 | 2.96 | 0.001 | EYS | −9.65 | 0.037 |
| MT-CO1 | 2.50 | 0.001 | SERAC1 | −9.65 | 0.037 |
| MT-CYB | 2.40 | 0.001 | TMEM184A | −9.43 | 0.001 |
| MT-ND5 | 2.29 | 0.001 | SENP2 | −9.39 | 0.044 |
| MT-ND3 | 1.91 | 0.001 | TSPAN31 | −9.31 | 0.001 |
| | | | UBE2N | −8.80 | 0.001 |
| | | | MPI | −8.64 | 0.001 |
| | | | TMEM123 | −8.62 | 0.002 |
| | | | SERPINE1 | −8.35 | 0.001 |
| | | | HACD4 | −8.13 | 0.004 |
| | | | NUDCD2 | −7.8 | 0.001 |

Top genes altered (up and down) in HUVECs growing on TiB2 substrates compared to cells growing on conventional plastic flasks (LEFT = Up-regulated, RIGHT = Down-regulated).

### Example 3—Discussion

There has been increased interest in identifying appropriate biomaterials and tissue culture substrates, especially platforms that enable 3D microenvironments, to meet the demands of the myriad of applications for tissue engineering, regenerative medicine, and drug discovery. The inventors have demonstrated a novel microfabricated substrate with the unique combination of biomaterials Si and TiB₂, ZrB₂ and HfB₂ for cellular patterning in tissue culture applications, without the need for additional biochemical surface modifications. Specifically, the use of commonly used culture media components such as endothelial cell growth supplement and heparin enable spatial patterning of endothelial cells, providing a 3D microenvironment for mesenchymal stem cells by promoting aggregation. Importantly, differences in the properties of the two biomaterials (Si and the mentioned above diborides) provide several beneficial cues for cell growth and differentiation.

Si is well established as a biomaterial and in this study, and the inventors demonstrate the potential of 4^{th} group transition metal diborides, TiB₂, ZrB₂ and HfB₂ for tissue culture applications. These diborides are characterized by extreme hardness, stiffness and strength, as well as high thermodynamic (melting at above ˜3,000° C. [124]) and chemical stability [29, 125-127]. They also have low electrical resistivity (TiB₂ 9-15 μΩcm, ZrB₂ 6.7-22 μΩcm HfB₂ 6.3-16.6 μΩcm) and high electron work function (˜5 eV) that affects its surface charge. Deposition of thin TiB₂, ZrB₂ and HfB₂ layers on Si was fabricated using e-beam evaporation rather than by sputtering, allowing for precise control of layer stoichiometry, due to phase diagrams of these diborides, and ensuring reproducibility. Selected aspects of cell substrate mechanisms were addressed here, to probe if TiB₂, ZrB₂ and HfB₂ have the potential to become biomaterials for cell culture. TiB₂, ZrB₂ and HfB₂ were used for specific in vitro cellular patterning of HUVECs and MSCs in this study. By patterning these diboride layers on Si, which is a softer material, the inventors created mechanical gradients of stiffness and hardness, observing their effect on cell growth and viability. TiB₂, ZrB₂ and HfB₂ on Si or SiO₂/Si behave as cell culture compatible materials that cells migrate onto due to durotaxis, elongating, spreading and proliferating on the patterns. Cell growth and alignment was also affected by micropattern dimensions and geometry, showing facilitated contact guidance in HUVECs and pattern-shape facilitated MSC aggregation. Preferential cell growth (relatively more cells on micropatterns versus background) was seen on patterns in the absence of growth factors and heparin, whereas in their presence growth was highly specific, restricted only to the geometric patterns and alignment was guided by pattern orientation. While narrow line widths caused HUVEC elongation, significantly larger pattern dimensions, larger than cells, did not control cell orientation apart from at edge regions.

The inventors' material characterization results implicate predominant mechanisms that lead to selectivity of cellular adhesion and growth. From XPS spectra the inventors identified stoichiometric TiB₂, ZrB₂ and HfB₂ in the bulk regions of the films and oxides composted of B, O and respective metal at the surface with varying concentrations and oxidation levels. All these diborides has strong susceptibility to oxidation especially at high temperatures [85-87, 173] where surface oxides grow [83] and are known to provide passivation and biocompatibility for implants. These oxides were present on the substrates as seen in cross-sectional TEM images. Additionally, boron oxide (B₂O₃) was found at the diborides surface, which has been shown to increase surface hydrophilicity [128], thus enabling cellular attachment.

There is increasing evidence that cell-surface interactions occur at multiple roughness scales from micrometers to nanometers [130]. AFM measurements confirmed TiB₂ patterns had slightly higher roughness compared to the Si background. Substrate roughness plays an integral part in cell-biomaterial interaction, altering a range of cellular functions from cell adhesion to morphology [129]. For this substrate, roughness effects are negligible as the measured topography of the background Si (1.6±0.1 Å) and TiB₂ (2.6±0.2 Å) are below the critical threshold ranges indicated in the literature for mammalian cell adhesion [98, 131]. Also noted from nanoindentation and contact angle measurements, the TiB₂ patterns showed increased hardness and hydrophilicity when compared to background Si. Biomaterial stiffness is known to impact cell adhesion, proliferation and differentiation [56, 108, 132, 133]. In the absence of media supplements (FIG. 5), for MSCs the inventors clearly noted preferential cell attachment to the hard TiB₂ patterns (14 GPa) compared to the Si background (10 GPa). This behavior conforms with published literature wherein MSCs exhibit durotaxis [57]. These material properties of the borides including their very high hardness and stiffness, chemical termination of the surface by oxides metal oxide and B₂O₃, and wetting behavior, provide favorable conditions for cell adhesion and growth. Such results were obtained in measurements of all analysed diborides TiB₂, ZrB₂ and HfB₂ deposited on the Si wafers.

Importantly, due to the differences in PZC of Ti and Si [78] and with additional modification of surface charge by B₂O₃, open circuit measurements confirmed that the TiB₂ patterns were less negatively charged both in DI water and ECGS and heparin supplemented media than the background Si. This change in surface potential may significantly contribute to protein adsorption, especially in the presence of heparin, which is a highly sulfated glycosaminoglycan with a very high negative charge density [134]. Heparin is known to bind to endothelial cell and fibroblast growth factors, increasing their affinity for the receptor while inducing a more mitogenic conformation [102, 111]. It is likely that the repulsive forces between the negatively charged heparin and the more negative surface charges of Si, restrict the adsorption of growth factors on Si. Indeed, this observation is confirmed by XPS and AFM studies that show decreased protein deposition on Si when compared to TiB₂. Another factor that may contribute to pattern specific cell selectivity is heparin's role in cell-fibronectin interactions in bone-derived stem cells. Heparin-binding domains within fibronectin are known for promoting cellular adhesion to fibronectin through interactions with cell surface heparan sulfated proteoglycan [135].

Overall substrate properties are one of a very broad spectra of environmental cues that influence cellular behavior. Mechanical properties such as stiffness, hardness, and elasticity lead to modulation of mechanotransduction, and are also known to be responsible for cell growth, motility and elongation [36] [136] [137]. Substrate physical properties such as topography, roughness, non-planarity, or flatness [138] [114, 139] have also been shown to affect cell attachment, elongation, and spreading. In addition, substrate chemical composition, electrical properties and surface free energy have been shown to be important modifiers of cellular interactions. Atomic termination modifications can render a surface hydrophobic or hydrophilic thus changing adsorption of various adhesion proteins. However, due to the complexity of these interactions, there are no predetermined material/cell interdependencies and any generalization of key mediators is impossible. Protein adsorption occurring in the early stages of cell-biomaterial interactions plays an important role in subsequent cell adhesion and spreading. Surface chemistry [41, 140, 141], wettability [142, 143], charge [144-146] and topography (roughness) [37, 38, 147, 148] are important influencing factors in the interactions at biomaterial surfaces [149]. Each of these surface properties are intricately linked, interacting synergistically or antagonistically and also presenting spatiotemporal interdependencies, making it difficult to ascertain a direct relationship between a single surface property and protein adsorption and/or subsequent cell adhesion [118, 33 140, 141, 143, 147, 149-158]. Significant changes in material surface energy, for the same degree of roughness, can initiate sharp transitions in wettability, from hydrophobic to hydrophilic and vice versa, with distinct cell absorption preference for moderate hydrophilic/hydrophobic surfaces [149]. Electrostatic interactions have also been shown to dominate cell adhesion [157, 159-162]. Similarly, different cell types have their own unique characteristics related to how they respond to the surface charges, wettability and free energy, roughness and chemical composition of surfaces [163-165]. For the inventors' substrate, roughness effects are negligible. With regards to wettability, Si when immersed into aqueous solutions can become hydrophilic due to the oxide present at its surface or can be hydrophobic when passivated by hydrogen (e.g., after Hydrofluoric acid etch). TiB₂, ZrB₂ and HfB₂ are more hydrophilic compared to Si due to the presence of boron oxide at the surface [166]. On the other hand, silicon with SiO₂ at its surface has lower electric potential (isoelectric point; IEP 2) than TiB₂, ZrB₂ and HfB₂ both in water and various media solutions, and TiB₂, ZrB₂ and HfB₂ with their larger electron work function values have surface potential less negative compared with Si as determined by OCP for TiB₂. Despite the relatively more hydrophilic nature of TiB₂, the inventors repeatedly detect slightly better protein adsorption to TiB₂ as compared to Si as evidenced by XPS (˜20% of the Si surface is visible in XPS compared to 10% of TiB₂ in the presence of FBS or ECGS without heparin). Accordingly, the inventors observe a higher number of cells adhering to TiB₂ compared to Si. However, in the presence of heparin and ECGS, protein adsorption to TiB₂ is homogenous and highly improved when compared to Si (˜74% of the Si surface is visible in XPS compared to 22% of TiB₂). Repulsive forces between the negatively charged heparin and the more negative surface charges of Si, may hinder the adsorption of heparin binding growth factors on Si. Additionally, heparin induces oligomerization of fibroblast growth factor molecules, facilitating its dimerization and activation when bound to the FGF receptor [167], and also stabilizes growth factor activity by preventing proteolytic degradation [168]. In summary, TiB₂, ZrB₂ and HfB₂ patterns exhibit multiple cues such as improved hydrophilicity, surface charge (less negative) and hardness when compared to Si which facilitate improved cell adhesion in the absence of heparin and growth-factors, but cellular patterning is only achieved in the presence of heparin and growth factors.

Here, the inventors have shown that TiB₂, ZrB₂ and HfB₂ as rigid material patterned on Si/SiO₂, improved adhesion of HUVECs, their proliferation and growth and led to selectivity in cell culture with supplemented media, whereas MSCs responded to the differences in the stiffness of the patterns and showed preferential growth on these borides in the absence of ECGS and heparin, and guided aggregation and formation of aggregates in the heparin and ECGS supplemented media.

For HUVECs, cell-spreading and attachment was restricted by the patterns, whereby the cells were observed to align and proliferate on TiB₂, ZrB₂ and HfB₂ line patterns and pattern edges, while random orientation and growth was observed within the circular patterns. Similarly, decreasing pattern line widths showed increased cell alignment, whereas thicker lines showed aligned cells along the edges and more random cell orientations within the center of the patterns. HUVEC mechanical response to the patterns was further corroborated via visualization of focal adhesion complexes and cytoskeleton, which indicated strong cell adhesion and actin fiber alignment to the patterns. Most importantly, the HUVECs were viable over 2-3 weeks in culture as indicated by cell counts, positive AO staining and expression of PECAM. These results are consistent with other studies, where patterning of TiO₂ showed increased adhesion of HUVECs [169], and previous studies on cell-pattern interaction have shown pattern-size and -shape dependent spreading and alignment of cells [67, 116, 118, 170]. Similarly, MSCs were viable over a culture period of 2-3 weeks and exhibited phenotypic stability as noted by CD105 and N-cadherin expression. Additionally, in accordance to their innate in vivo behavior, MSCs exhibited mobility on the patterned surfaces in response to the stiffness gradient imparted by the hard borides layers on the relatively less stiff Si background. Importantly, circular patterns enabled MSC 3D aggregate formation, which is important in providing a 3D microenvironment, especially in applications that target cell differentiation.

Epithelial ovarian cancer (EOC) is one of the most lethal cancers for women in the US, with a majority of women presenting at advanced stages, when survival rates are poor [188]. Despite rapid advances in surgical debulking, platinum-based chemotherapy, targeted agents and immunotherapy, relapse and chemoresistant metastasis continue to drive EOC's high mortality rates. Thus, there is a critical need for advancing the understanding of molecular mechanisms related to the migratory and invasive properties of ovarian cancer cells.

Tumorigenesis is a complex multi-faceted process orchestrated via an interplay of key phenomena, including but not limited to, epithelial to mesenchymal transition (EMT), tumor initiating cancer stem cells (TIC), metastasis competency and development of chemoresistance. EMT has been shown to be essential for acquisition of invasive metastatic properties [189] and inducing TICs in ovarian cancer [190]. Both TICs and metastasis are also intricately linked with resistance to chemotherapy [191, 192]. Notably, it is now known that in addition to soluble signals such as growth factors and cytokines which can stimulate EMT, biomechanical forces act as stimuli that also modulate the morphology, expression of biomarkers, and aggressiveness of tumor cells [193]. As such, there is now considerable interest to better understand how cells sense physical forces and convert “mechanical signals” into biological responses, with concomitant interest towards the development of mechanobiology-targeted therapeutic approaches (known as mechanopharmacology) for cancer [192, 194].

Although challenging, this landscape of related factors (EMT, TICs, metastasis, biomechanics) needs to be unraveled via in depth research to provide insight into cancer progression and treatment response [195]. For ovarian cancer, this landscape is exacerbated by the multi-faceted heterogeneity of EOC. Not only does EOC have distinct histological subtypes, but there is also intra-tumor heterogeneity among the cell populations, and there is molecular heterogeneity at the clinical level amongst individual patients and patient populations [196]. Finally, chemotherapeutic treatments can further drive tumor evolution, inducing further heterogeneity at various levels [197].

Current platforms for evaluating factors influencing tumorigenesis have included in vitro strategies involving conventional two-dimensional (2D) monolayer tissue culture, three-dimensional (3D) culture systems, transwell assays, and in vivo animal models with syngeneic, xenografts, and patient derived (PDX) tumor models. While 2D systems fail to mimic in vivo 3D microenvironments, animal models are expensive [198]. 3D in vitro systems based on spheroids, aggregates and organoids have provided a cheaper and biological relevant alternative, but majority of current systems suffer from handling limitations, lack reproducibility in terms of spheroid dimensions and shape, and do not provide direct accessibility to probe surface properties [199]. Most importantly, the 3D cancer cell spheroid models are not conducible to monitoring the metastasis competency of cells in terms of identification and quantitation of the migratory behavior of cells arising from the spheroid periphery.

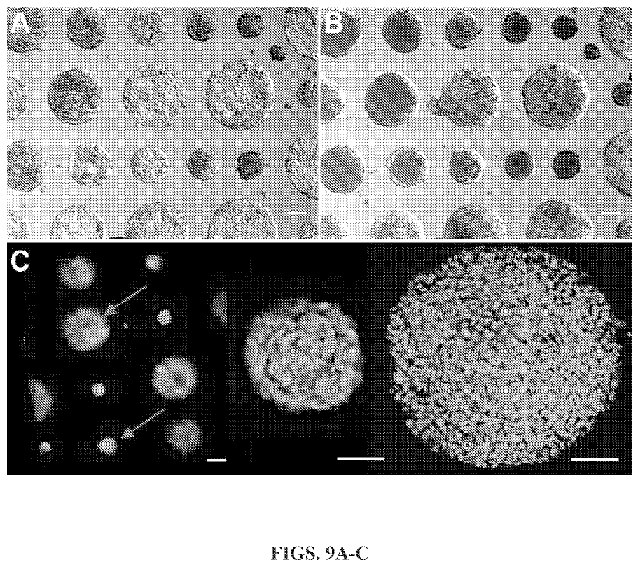

Interestingly, similarly to MSCs, in the presence of growth factors and heparin, 3D aggregation was also noted for human fibroblasts and ovarian cancer SKOV3 cells. Based on this observation, the inventors have developed a novel silicon-diboride (Si—TiB₂, Si—ZrB₂, Si—HfB₂) micropatterned substrates which overcome this barrier [200]. On this substrate, 3D aggregates grow on circle patterns, enabling direct access to 3D aggregates of tumor cells. As shown in FIGS. 9A-C, EOC cell line SKOV3 forms 3D aggregates that reach a thickness of 60-80 μm (95% CI of 55.75-63.06) by day 7 after seeding. 3D aggregate diameter depends on circle pattern diameter (p<0.05) [201]. This mass-producible 2D platform that enables 3D culture is compatible with a palette of single/small cell analysis techniques to reveal targetable cell and molecular mechanisms, crucial for probing complex events in tumorigenesis and facilitate the development of targeted therapies and diagnostic tools.

This substrate provides a unique experimental setting with well-characterized salient features (roughness, stiffness, wettability, and charge) that enables simultaneous observations of cell behavior in monolayer versus 3D aggregates under similar biochemical and physical cues [200]. The inventors have established the feasibility to assess tumorigenesis in EOC 3D aggregates formed on the inventors' substrates, specifically investigating behaviors that drive early metastatic cell differentiation. Preliminary studies indicate that from an array of EOC 3D aggregates generated on the inventors' substrates, 13% exhibit relatively higher invasiveness leading to the development of inter-aggregate multicellular bridges. These “cellular bridges” are reflective of adaptive processes occurring in a sub-population of 3D aggregates, within which, cells develop higher metastasis competency that drives their migratory behavior. The result of which is cells bridging gaps across neighboring aggregates to develop multicellular channels.

This novel low-cost Si-diboride substrates enable arrays of 3D cancer cell aggregate cultures, and can be used as a discovery platform for (a) the identification of novel targets in the adaptive transition of cells to attain metastasis competency, and (b) examining the efficacy of targeted EOC therapy currently in clinical trials.

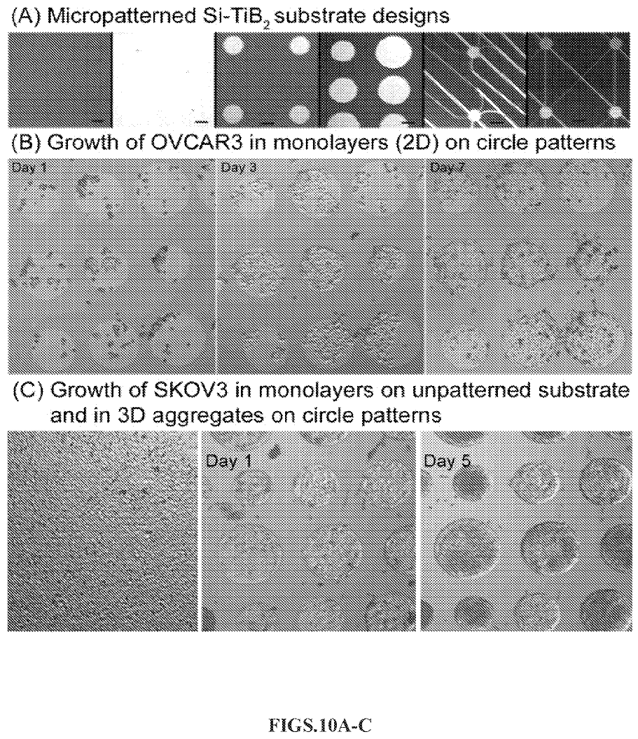

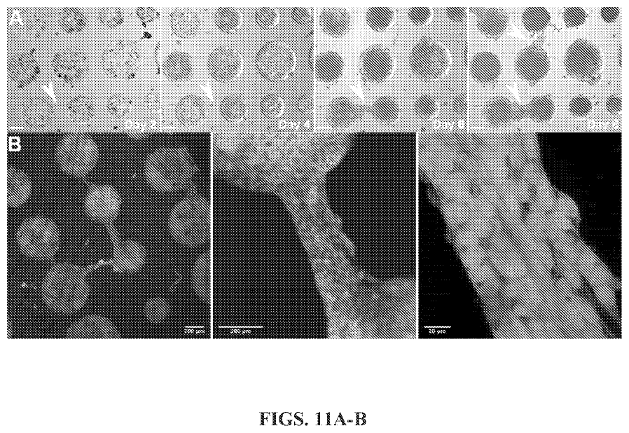

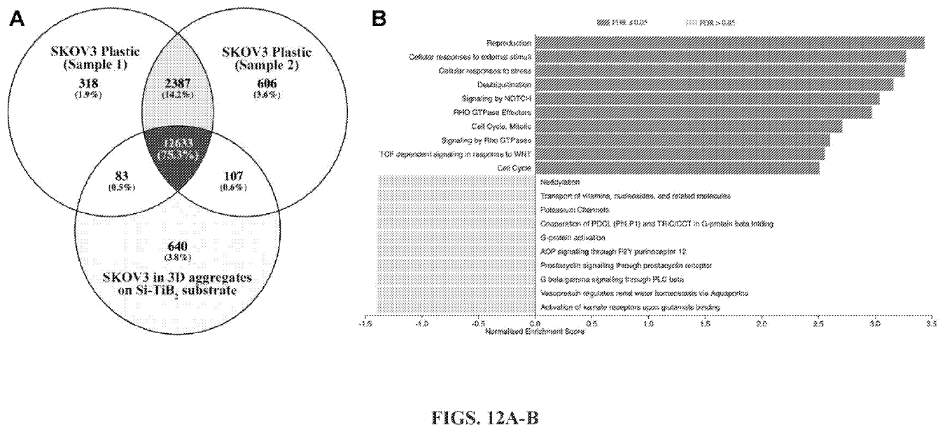

The Si-diboride substrate can be used to significantly impact the understanding of ovarian cancer growth and treatment via optimized substrate designs that leverage substrate properties, such as stiffness gradients (known to promote durotaxis). Innovations include that the micropatterned substrate consisting of two biocompatible biomaterials of varying stiffness: silicon; Si and titanium diboride; TiB₂ (FIG. 10A FIG. 10A-), wherein TiB₂ is micropatterned on the Si to create specific geometric designs and tune substrate topology [200]. Micropatterning has been used with other materials to elicit effects of contact guidance [202, 203], however, there has not been a similar Si—TiB₂ substrate created for cancer research to date that enables customizable pattern geometry permitting simultaneous but separate regions of monolayer or 3D culture on a single substrate, allowing observation of both under similar biochemical and physical cues. As seen in (FIGS. 10A-), these substrates allow pattern specific monolayer (2D) versus 3D culture of different cells. As expected [204], low invasive potential EOC cell line OVCAR3 over a week in culture, preferentially grows on the TiB₂ micropatterns in monolayers but fails to form 3D aggregates (FIGS. 10A-), whereas highly invasive EOC cancer cell line, SKOV3 cells grow in monolayers on unpatterned TiB₂ (left panel, FIGS. 10A-) and self-assemble into 3D aggregates around days 3-4 (right panel, (FIGS. 10A-10C) to form tightly compacted 3D aggregates (days 5-7) over time. The behavior cells on the anologus ZrB₂ and HfB₂ patterns formed on Si wafers was the same as on TiB₂ patterns.

Importantly, these substrates can be used to monitor genomic adaption to migratory profiles in cancer cells. While previous studies have established the importance of mechanotransduction on EOC, current 3D spheroid culture methods cannot be used to delineate the adaptive transcriptomic landscape that supports migratory cancer cell behavior. These novel Si—TiB₂ substrates allows us to create mechanotransducive environments through micropatterned stiffness gradients.

The 3D aggregates of SKOV3 (high invasiveness EOC) formed on the inventors' substrates enable observation of metastatic behaviors such as cell migration and development of inter-aggregate “cellular bridges” (white arrows in FIG. 11). The technology and outcomes from this study are not limited to ovarian cancer and may be utilized to drive further advancements to other diseases. The inventors have also demonstrated the feasibility of the inventors' substrate to assess tumorigenesis in 3D aggregates formed on the inventors' substrates, using EOC cancer cell lines of varying invasive potential and chemosensitivity or chemoresistance [204].

Transcriptome analysis of patient samples at multi-stage disease progression from primary EOC to distal metastatic sites serve as the foundational resource for the understanding of ovarian cancer. Unfortunately, inter-tumor heterogeneity prevents the leveraging of this knowledge for EOC prognosis. Patient-derived xenografts (PDX) are promising pre-clinical models but, PDX requires large surgical samples and costly in vivo animal studies [205]. 3D culture systems provide a viable alternative to animal models [206, 207, 208], but most do not define the adaptive progression of the disease; instead, current cell-systems enrich for select endpoint EOC cells populations (e.g., single-cell vs. aggregate-forming, invading vs. non-invading). Hence, independent cellular tools are frequently used concurrently to assess the dynamic cancer cell biology. Notably, generation of comparative inferences from the large cohort of in vitro cell-based platforms used in cancer biology has been challenging due to the diversity of tools and lack of standardization [199]. To overcome this, the inventors will utilize the Si—TiB₂ substrates that the inventors developed [200, 201] to characterize 3D aggregates with static versus migratory phenotypes. Studies have established that the aggressive SKOV3 cells are mesenchymal-like, while OVCAR3 are more epithelial-like cells [204, 209]. Also, SKOV3, unlike OVCAR3, form tumors when injected subcutaneously and intraperitoneally into nude mice [210, 211]. As shown in FIGS. a unique attribute of these substrates is that depending on the invasive potential of cells, their ability to form 2D monolayers (OVCAR3 on any patterns or SKOV3 on planar patterns) or 3D aggregates (SKOV3 on circular patterns), and the size and number of the 3D aggregates can be controlled with simple customization of photolithography masks. The inventors performed total RNA-seq analysis of SKOV3 aggregates (day 7) on the Si—TiB₂ substrates (FIGS. 12A-B). STAR alignment [212] of the transcripts from substrate with controls from plastic (NCBI GEO: GSM5049693) show >75% overlap, with 640 (3.8%) unique transcripts for the substrate (FIGS. 12A-B). Notably, over-representation analysis of these 640 transcripts was not statistically significant, indicating that the inventors' substrates maintain SKOV3 genotype. Next, they performed gene-set enrichment pathway analysis (GSEA) for up-regulated genes with IFC>|2| [213]. FIGS. 12A-BB shows the top up-regulated pathways for SKOV3 on substrates. Interestingly these pathways highlight significant up regulation of Wnt signaling and TCF dependent signaling downstream of B-catenin binding [214], alongside upregulated RHO GTPase signaling—both processes shown to regulate cell motility and invasion [215].

The inventors used their Si-diborides substrates (described in [200]) for culture of SKOV3 (ATCC® HTB-77™) cancer cells of high invasive potential. Cells were seeded on Si—TiB₂ substrates (2×2 cm) at a density of 600/mm², and each substrate will be placed in a well of a 24-well dish and cultured in RPMI 1640 with glutamine, 20% FBS, 1% antibiotics, 0.01 mg/mL Insulin, 10 ng/ml FGF2 and 1% heparin 1 over 10 days to establish a monolayer (days 1-3) or allowed to form 3D aggregates (days 4-10). A 2×2 cm substrate can accommodate at least 500, 300 μm circle patterns (aka 500 3D aggregates of ˜150-200 μm diameter and 60-80 μm thickness). Based on preliminary data, the rate of “cellular bridge” formation is 13%/substrate or 50%/aggregate. As shown in FIGS. 9A-C, FIGS. 10A-C and FIG. 11A-B, highly invasive SKOV3 begin as monolayers and then self-assemble to form 3D aggregates. Importantly, the inventors are able to control dynamics of aggregation and size and number of aggregates by tuning pattern geometry and cell seeding density [201]. 3D aggregates of static (compact aggregates without any migratory sequalae) and migratory phenotype (identified via cells migrating away from an aggregate and establishing connections with neighboring aggregates; see FIGS. 12A-B) can be identified post-culturing at day 2, day 4, day 6 and day 8. These preliminary results suggest self-assembly and aggregate formation is complete around days 3-5, and the majority of bridge formations occur around day 6. Thus, these capture time points have data on cell monolayers (Day 2), self-assembly (Days 3-4) and compact 3D aggregates (Days 5-8). Migratory 3D aggregate phenotypes are visible at Days 5 with maximum numbers of cellular bridges across aggregates seen at Days 6-7. Migratory 3D aggregates can be scraped off substrates when using substrates with multiple 2 aggregates, or just digested off from substrates with customized singular pattern geometry for one pair of aggregates. FACS-enriched, deep coverage scRNA-seq can then be performed on cells from each of the 4 time-points to establish “snapshots” of the dynamic adaption of EOC cells.

Effect of targeted therapy on migratory phenotypes in 3D aggregates using Si—TiB₂ substrate. Acquired and intrinsic resistance to standard chemotherapy is common in EOC patients. In fact, chemo-resistance is responsible for the onset of incurable metastatic disease and low survival rates in patients with high-grade EOC. Hence an overarching challenge remains to identify targeted stand-alone or combinatorial therapy for advanced ovarian cancers. As chemotherapy leads to an induction in histone deacetylase (HDAC [216], HDAC inhibitors (HDACi) including pan-genome suberoylanilide hydroxamic acid (SAHA, aka Vorinostat) [217] emerge as promising therapy. The inventors have monitored the effect of HDACi molecules with and without platinum-based chemotherapy to monitor their effect of EOC metastasis potential. SAHA, in combination with platinum-based chemotherapy, is currently in clinical trials for ovarian cancer patients with recurrent and/or progression to metastatic disease. Since pre- and post-metastasis recurrent ovarian cancers are distinct and exhibit unique drivers [218], the Si—TiB₂ substrate can be used for monitoring treatment response. The substrates can be used in preclinical screening to identify drug-therapy responders from a mixed clinical cohort of pre- and post-metastasis recurrent ovarian cancers.

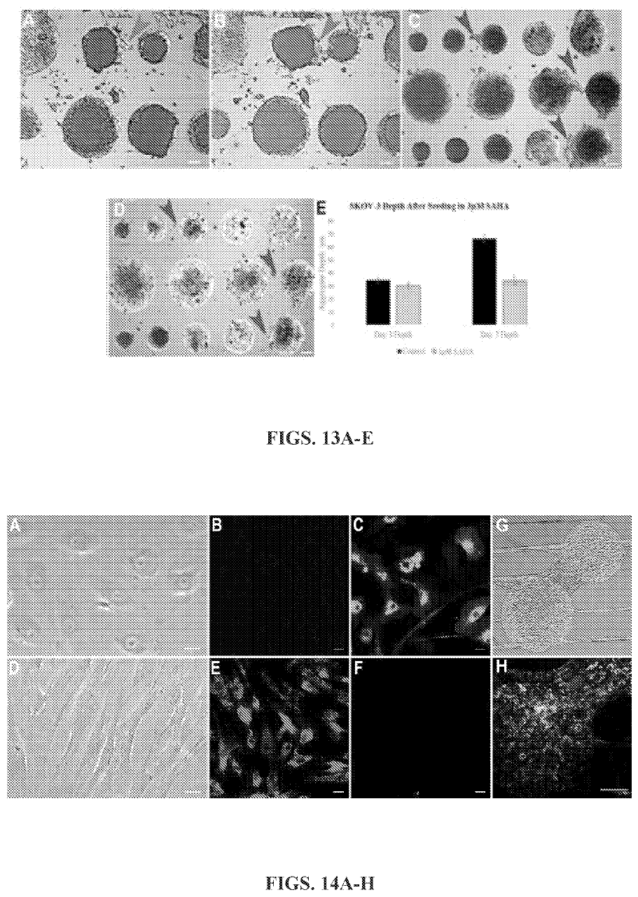

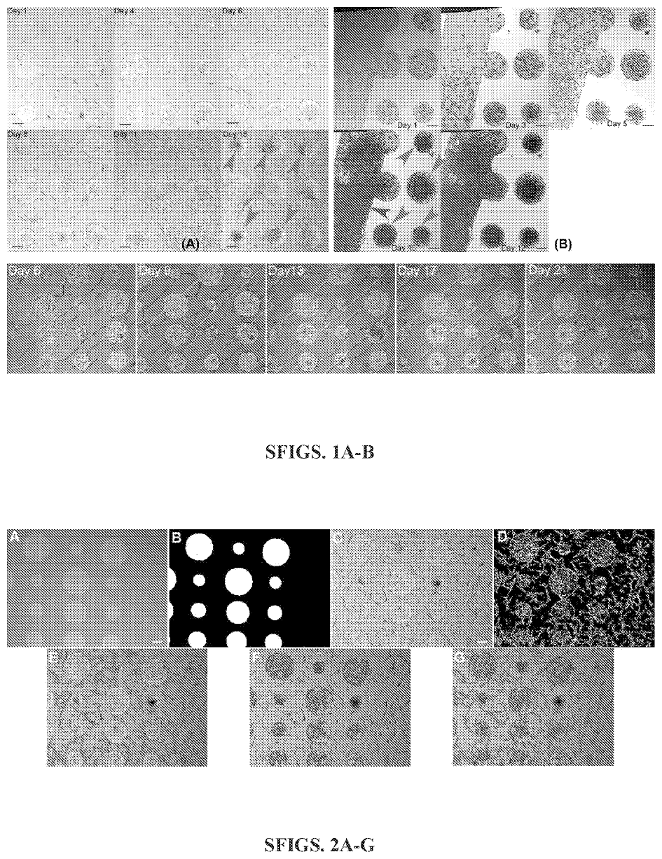

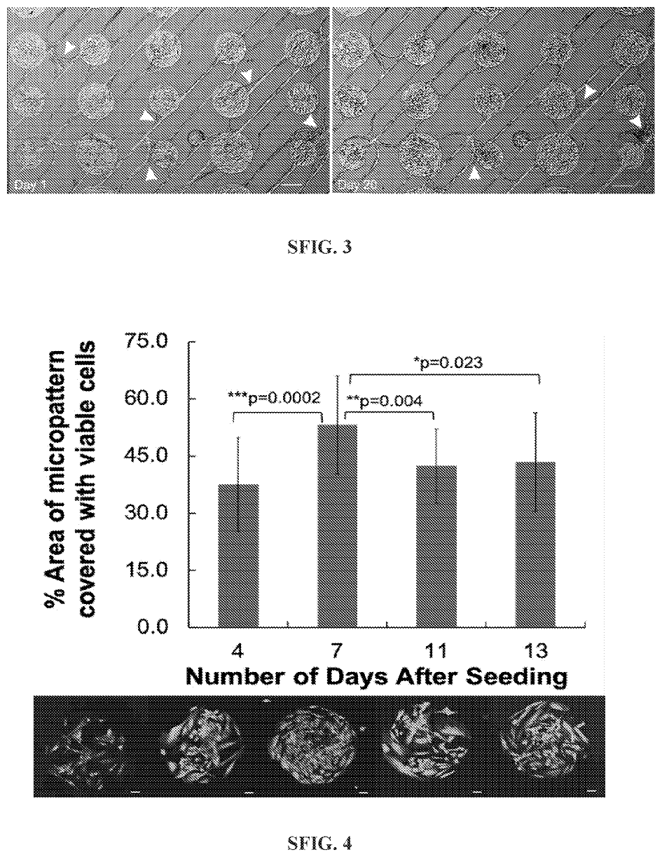

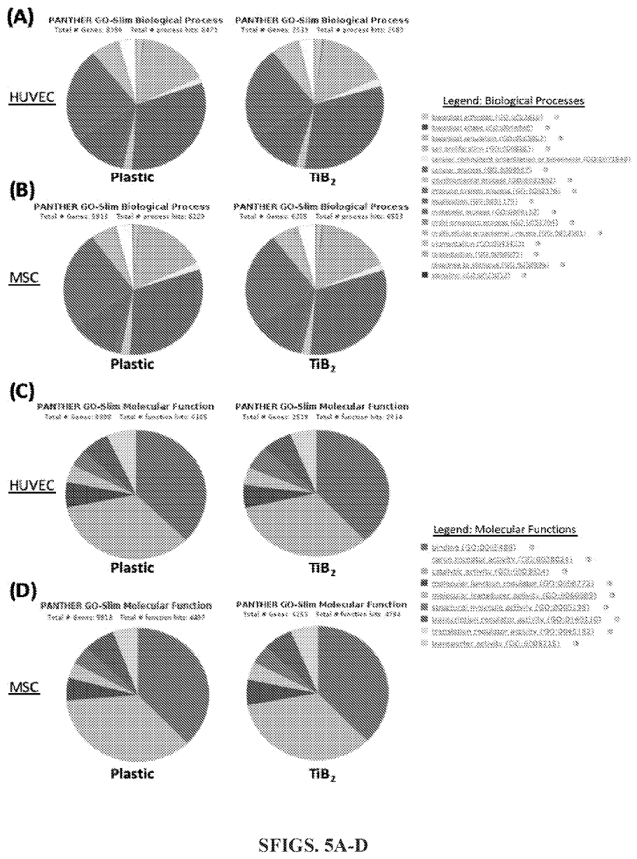

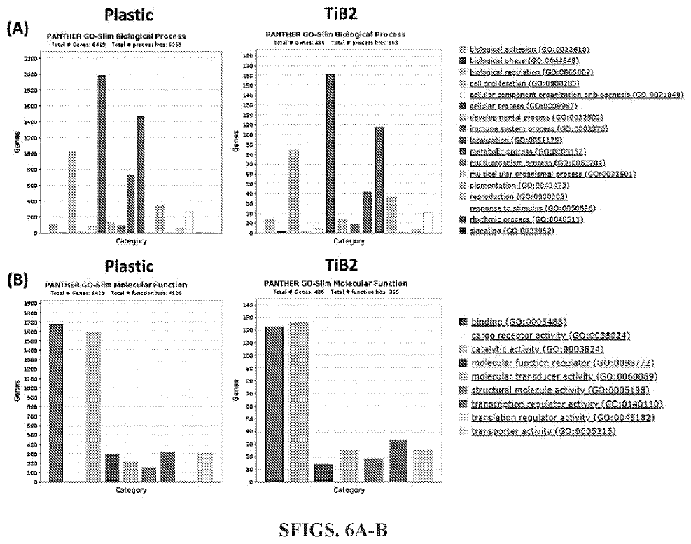

Preclinical drug screening platforms are lacking that recapitulate migratory behavior of EOC cells. Hence, the Si-diborides substrate present a unique opportunity for use as a future screening platform. Microfabrication of circle patterns in predefined arrays of known sizes make the substrate amenable for screening via automated imaging scripts. Also, cell motility (as seen in FIG. 11) is quantifiable with emergence of cellular bridge micro-structure. Chemoresistant SKOV3 cells were cultured on the Si—TiB₂ substrate for 7 days to initiate formation 3D aggregates and the invading cellular architecture (FIGS. 13A-EA) mimicking microinvasion. At this point, Si—TiB₂ cultured cells were treated with sublethal doses of 3 μM SAHA [219, 220]. While untreated aggregates continue to grow in size and begin to develop cellular bridges (FIGS. 13A-EB, arrows), treatment of cells on day 7 post-seeding with SAHA disassociates invading multicellular microstructures (FIGS. 13A-ED, arrows) and reduce the size of 3D aggregates (FIGS. 13A-E 13E).

Further validation of the Si-diborides approach for the investigation of cancer metastasis and EOC growth and motile dynamics is observed through upregulated deubiquitination (FIGS. 12A-B) [221, 222]. Transcriptome analysis of EOC SKOV3 3D aggregates indicates induction of the deubiquitination pathway with elevated mRNA levels of multiple DUBs including UCHL1, UCHL3, A20/OTUD7c, and OTUB2 (FIGS. 12A-B). Like HDACi, targeting deubiquitination enzymes (DUB) is also emerging as a promising therapy for advanced EOC. Induction of DUB deregulate protein degradation to drive chemoresistance in multiple endocrine-driven cancers [223, 224] including ovarian cancer [225]. Genomic knockdown of DUB USP39 in ES2 restored chemo-sensitivity while concurrently reducing cell migration and invasion[226]. Similarly, a small molecule RA-9 reduces activity of proteasome-associated DUB (USP2, USP5, USP8, UCHL1, UCHL3, and UCHL5 in Sigma, MSDS) to increase ubiquitin-mediated protein degradation and decreases viability of chemoresistant EOC cells SKOV3, and OVCAR3 [227]. Additionally, EOC ES2 tumors in xenografts were significantly inhibited with administration of the same small molecule. DUB inhibitors can be tested for their effect on EOC metastasis or even EOC cell motility/invasion. The Si—TiB₂ system presents a unique platform to test the efficacy/potency of targeted therapy against EOC cell migration.

Co-culture of mesenchymal stem cells and human umbilical vein endothelial cells using the Si-diboride substrates. The Si-diboride substrate supports cellular patterning and enables monoculture or co-culture 3D microenvironments. The substrate offers control on aggregate size while simplifying handling of cell aggregates and providing direct access to cells for assessment via optical imaging and standard contact-based technologies such as AFM used to measure mechanical properties. These are distinct advantages over the 3D culture techniques for generating spheroids such as ultra-low attachment plates, hanging drop, micro-wells, and use of natural and synthetics gels. Additionally, the substrate enables co-culture of cells, as shown for mesenchymal stem cells (MSCs) and human umbilical vein endothelial cells (HUVECs) as shown for TiB₂ in FIGS. 6A-H.

RNA-seq transcriptome analysis confirmed sustained metabolic and biological activity of cells cultured on the micropatterned substrates in comparison to conventional monolayer culture in plastic flasks. Despite, the robust molecular and biological profiles observed across the cells on the substrate versus those on plastic, the inventors observed a reduction in the total number of transcripts identified for HUVECs cultured on the substrate. In order to gain additional insight into the disparity of the number of genes expressed for HUVECs, the inventors compared their RNA-sequencing data with existing data on the NCBI's Gene Expression Omnibus (GEO) archives. The inventors retrieved five datasets of total RNA-sequencing of HUVECs grown on tissue culture plastic (TCPS) coated with gelatin: GSM3494325 (TCPS_control_Rep1 and TCPS_control_Rep1), GSM1828760 (SCR_static_n2), GSM1828761 (SCR_static_n4) and GSM1828762 (SCR_static_n5). The number of expressed genes was consistent (>10,000 transcripts) across samples, specifically; 12993 genes in TCPS_control_Rep1, 10202 genes in TCPS_control_Rep2, 10,476 genes in SCR_static-n2, 10755 genes in SCR_static-n4, and 13166 genes for SCR_static-n5. Considering methodological differences, the number of transcripts (8295) identified for the inventors' HUVEC dataset cultured on tissue culture plastic coated with gelatin, is in agreement with archived data on NCBI's GEO. Furthermore, the inventors compared the list of genes from the GEO datasets and these data and obtained percentages of genes shared between pairs of datasets (e.g., SCR_static-n2 vs. the inventors' data), confirming 65-75% of shared gene signatures. These results validate the inventors' RNA-sequencing data and give sufficient confidence in the data set.

Transcriptome analysis for HUVECs grown on TiB₂ patterns is unique to this study and the inventors were unable to locate any datasets on total RNA-sequencing of HUVECs on titanium or its alloys on the NCBI GEO archives. In the absence of published data on the total number of transcripts recorded for HUVECs on different biomaterials in existing literature, the inventors performed transcriptome analysis and identified differentially expressed genes on plastic controls versus their substrates, and performed PANTHER classification analysis, which confirmed that both cell populations displayed stable biological and molecular functions. For HUVECs grown on plastic and those grown on the TiB₂ substrates, the overall profiles for GO term mappings for biological and molecular functions for the unique set of genes expressed on plastic only versus those expressed on the substrate, are similar (SFIGS. 6A-B). While showing that the combination of biomaterials Si and TiB₂, ZrB₂ and HfB₂ is appropriate for proper cell growth and aggregation, pathway analyses using PANTHER software also identified a set of processes that are differentially enriched between samples, further validating the inventors' previous observations. Specifically, genes involved in the regulation of cell adhesion such as cadherin (cell-cell) and integrin (cell-extracellular matrix) were over-represented in HUVEC cells grown on TiB₂, which agrees with the inventors' visualization of increasing focal adhesion complexes and cytoskeletal rearrangements. Furthermore, pathway analyses suggest a relationship between the diborides substrate growth and incremented metabolic activities, which may be associated with the ability of MSCs to aggregate.

In this study, the inventors demonstrate the novel combination of Si and TiB₂ ZrB₂ and HfB₂ patterned substrates for selective spatial patterning of cells in culture. This microfabrication process is not only amenable to scale-up but is also simple due to the absence of complex surface modification processes, making it attractive for large scale manufacturing. Moreover, the inventors tested tissue engineering micropatterns of biological relevance with defined geometries such as circles and lines, using HUVECs and MSCs, both of which play a critical role in tissue repair and regenerative medicine. This micropatterned substrate supports extended cell growth in culture, providing a viable tissue culture platform. Additional advantages include, (1) substrate reusability by simple removal of adsorbed proteins (2) control over the size, number, and uniformity of MSC aggregates generated via a simple microfabrication mask design (3) easy retrieval of MSC aggregates via gentle shaking or scraping and (4) micropatterns designed in defined arrays can enable systematic and reproducible evaluation (e.g., automating imaging) of aggregates. Although in this study the inventors demonstrated the role of endothelial cell growth supplement and heparin in directing pattern specific HUVEC and MSC growth, they note that cellular patterning is achieved by supplementing media with any heparin-binding growth factors such as FGF, VEGF, IGF or EGF. In their absence, cells exhibit preferential attachment to TiB₂ patterns versus Si, whereas highly selective growth to patterns is observed in supplemented media. The differential adsorption of proteins mediated by differences in the surface properties of Si and the diborides plays a key role in driving cellular patterning. In summary, the inventors have presented a novel microfabricated platform for cellular patterning and in vitro cell culture that will provide a powerful tool for potential applications in tissue engineering and drug discovery.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. References

The following references, to the extent that they provide exemplary procedural or other details supplementary to those set forth herein, are specifically incorporated herein by reference.
1. Langer, R. and D. A. Tirrell, Designing materials for biology and medicine. Nature, 2004. 428(6982): p. 487-92.
2. Martinez-Rivas, A., et al., Methods of Micropatterning and Manipulation of Cells for Biomedical Applications. Micromachines (Basel), 2017. 8(12).
3. Curry, E. J., et al., 3D nano- and micro-patterning of biomaterials for controlled drug delivery. Ther Deliv, 2017. 8(1): p. 15-28.
4. Derkus, B., Applying the miniaturization technologies for biosensor design. Biosens Bioelectron, 2016. 79: p. 901-13.
5. Ermis, M., E. Antmen, and V. Hasirci, Micro and Nanofabrication methods to control cell-substrate interactions and cell behavior: A review from the tissue engineering perspective. Bioact Mater, 2018. 3(3): p. 355-369.
6. Park, T. H. and M. L. Shuler, Integration of cell culture and microfabrication technology. Biotechnol Prog, 2003. 19(2): p. 243-53.
7. Sung, C. Y., et al., Integrated Circuit-Based Biofabrication with Common Biomaterials for Probing Cellular Biomechanics. Trends Biotechnol, 2016. 34(2): p. 171-86.
8. Falconnet, D., et al., Surface engineering approaches to micropattern surfaces for cell-based assays. Biomaterials, 2006. 27(16): p. 3044-3063.
9. Turner, A. M., et al., Attachment of astroglial cells to microfabricated pillar arrays of different geometries. J Biomed Mater Res, 2000. 51(3): p. 430-41.
10. Zahor, D., et al., Organization of mesenchymal stem cells is controlled by micropatterned silicon substrates. Materials Science and Engineering: C, 2007. 27(1): p. 117-121.
11. Yang, C. Y., et al., Micropatterning of mammalian cells on inorganic-based nanosponges. Biomaterials, 2012. 33(20): p. 4988-97.
12. Oshida, Y., 6-Biological Reactions, in Bioscience and Bioengineering of Titanium Materials (Second Edition). 2013, Elsevier: Oxford. p. 139-168.
13. Park, Y.-J., et al., Surface characteristics and bioactivity of oxide film on titanium metal formed by thermal oxidation. Journal of Materials Science: Materials in Medicine, 2007. 18(4): p. 565-575.
14. Chen, J. Y., et al., Behavior of cultured human umbilical vein endothelial cells on titanium oxide films fabricated by plasma immersion ion implantation and deposition. Surface and Coatings Technology, 2004. 186(1-2): p. 270-276.
15. Peddi, L., R. Brow, and R. Brown, Bioactive borate glass coatings for titanium alloys. Journal of Materials Science: Materials in Medicine, 2008. 19(9): p. 3145-3152.
16. Anabtawi, M., P. Beck, and J. Lemons, Biocompatibility testing of simulated total joint arthroplasty articulation debris. J Biomed Mater Res B Appl Biomater, 2008. 84(2): p. 478-85.
17. Makau, F. M., et al., Viability of Titanium-Titanium Boride Composite as a Biomaterial. ISRN Biomaterials, 2013. 2013: p. 1-8.
18. Sivakumar, B., R. Singh, and L. C. Pathak, Corrosion behavior of titanium boride composite coating fabricated on commercially pure titanium in Ringer's solution for bioimplant applications. Mater Sci Eng C Mater Biol Appl, 2015. 48: p. 243-55.
19. Hakki, S. S., B. S. Bozkurt, and E. E. Hakki, Boron regulates mineralized tissue-associated proteins in osteoblasts (MC3T3-E1). Journal of Trace Elements in Medicine and Biology, 2010. 24(4): p. 243-250.
20. Nielsen, F. H., The emergence of boron as nutritionally important throughout the life cycle. Nutrition, 2000. 16(7-8): p. 512-4.
21. Wu, C., et al., Proliferation, differentiation and gene expression of osteoblasts in boron-containing associated with dexamethasone deliver from mesoporous bioactive glass scaffolds. Biomaterials, 2011. 32(29): p. 7068-7078.
22. Gümüşderelio\check{g}lu, M., et al., Encapsulated boron as an osteoinductive agent for bone scaffolds. Journal of Trace Elements in Medicine and Biology, 2015. 31: p. 120-128.
23. Brown, R. F., et al., Effect of borate glass composition on its conversion to hydroxyapatite and on the proliferation of MC3T3-E1 cells. Journal of Biomedical Materials Research Part A, 2009. 88A(2): p. 392-400.
24. Ponomarev, V. A., et al., Microstructure, chemical and biological performance of boron-modified TiCaPCON films. Applied Surface Science, 2019. 465: p. 486-497.
25. Huang, Q., et al., SaOS-2 cell response to macro-porous boron-incorporated TiO2 coating prepared by micro-arc oxidation on titanium. Materials Science and Engineering: C, 2016. 67.
26. Wang, Y., X. Xue, and H. Yang, Synthesis and Antimicrobial Activity of Boron-doped Titania Nanomaterials. Chinese Journal of Chemical Engineering, 2014. 22(4): p. 474-479.
27. Song, Y. J., J. H. Choi, and H. Lee, Setdbl is required for myogenic differentiation of C2C12 myoblast cells via maintenance of MyoD expression. Mol Cells, 2015. 38(4): p. 362-72.
28. Attar, H., et al., Mechanical behavior of porous commercially pure Ti and Ti—TiB composite materials manufactured by selective laser melting. Materials Science and Engineering: A, 2015. 625: p. 350-356.
29. Morsi, K. and V. V. Patel, Processing and properties of titanium-titanium boride (TiBw) matrix composites—a review. Journal of Materials Science, 2007. 42(6): p. 2037-2047.
30. Sarma, B. and K. Ravi Chandran, Recent advances in surface hardening of titanium. JOM Journal of the Minerals, Metals and Materials Society, 2011. 63(2): p. 85-92.
31. Attar, H., et al., Comparative study of microstructures and mechanical properties of in situ Ti—TiB composites produced by selective laser melting, powder metallurgy, and casting technologies. Journal of Materials Research, 2014. 29(17): p. 1941-1950.
32. Ravi Chandran, K. S. and D. B. Miracle, Titanium-boron alloys and composites: Processing, properties, and applications. Jom, 2004. 56(5): p. 32-33.
33. Thery, M., Micropatterning as a tool to decipher cell morphogenesis and functions. Journal of Cell Science, 2010. 123(24): p. 4201-4213.
34. Chen, C. S., et al., Geometric control of cell life and death. Science, 1997. 276(5317): p. 1425-8.
35. Underhill, G. H., et al., Bioengineering methods for analysis of cells in vitro. Annu Rev Cell Dev Biol, 2012. 28: p. 385-410.
36. Ingber, D. E., From cellular mechanotransduction to biologically inspired engineering: 2009 Pritzker Award Lecture, BMES Annual Meeting Oct. 10, 2009. Ann Biomed Eng, 2010. 38(3): p. 1148-61.
37. Biela, S. A., et al., Different sensitivity of human endothelial cells, smooth muscle cells and fibroblasts to topography in the nano-micro range. Acta Biomaterialia, 2009. 5(7): p. 2460-2466.
38. Gonzalez-Garcia, C., et al., Effect of nanoscale topography on fibronectin adsorption, focal adhesion size and matrix organisation. Colloids Surf B Biointerfaces, 2010. 77(2): p. 181-90.
39. Grinnell, F., Focal adhesion sites and the removal of substratum-bound fibronectin. J Cell Biol, 1986. 103(6 Pt 2): p. 2697-706.
40. Rezek, B., et al., Micro-pattern guided adhesion of osteoblasts on diamond surfaces. Sensors (Basel), 2009. 9(5): p. 3549-62.
41. Steele, J. G., et al., Attachment of human bone cells to tissue culture polystyrene and to unmodified polystyrene: the effect of surface chemistry upon initial cell attachment. J Biomater Sci Polym Ed, 1993. 5(3): p. 245-57.
42. Gonzalez-Garcia, C., et al., The strength of the protein-material interaction determines cell fate. Acta Biomater, 2018. 77: p. 74-84.
43. Belair, D. G., N. N. Le, and W. L. Murphy, Design of growth factor sequestering biomaterials. Chem Commun (Camb), 2014. 50(99): p. 15651-68.
44. Ahn, K., et al., Features of microsystems for cultivation and characterization of stem cells with the aim of regenerative therapy. Stem cells international, 2016. 2016.
45. Lei, Y., et al., Geometrical microfeature cues for directing tubulogenesis of endothelial cells. PLoS One, 2012. 7(7): p. e41163.
46. Wang, W., et al., 3D spheroid culture system on micropatterned substrates for improved differentiation efficiency of multipotent mesenchymal stem cells. Biomaterials, 2009. 30(14): p. 2705-15.
47. Wang, X., et al., Regulating the stemness of mesenchymal stem cells by tuning micropattern features. Journal of Materials Chemistry B, 2016. 4(1): p. 37-45.
48. Rao, R. R. and J. P. Stegemann, Cell-based approaches to the engineering of vascularized bone tissue. Cytotherapy, 2013. 15(11): p. 1309-22.
49. Mao, A. S. and D. J. Mooney, Regenerative medicine: Current therapies and future directions. Proc Natl Acad Sci USA, 2015. 112(47): p. 14452-9.
50. Sun, X., W. Altalhi, and S. S. Nunes, Vascularization strategies of engineered tissues and their application in cardiac regeneration. Adv Drug Deliv Rev, 2016. 96: p. 183-94.
51. Melchiorri, A. J., B. N. Nguyen, and J. P. Fisher, Mesenchymal stem cells: roles and relationships in vascularization. Tissue Eng Part B Rev, 2014. 20(3): p. 218-28.
52. Shen, Y., et al., Biomaterial and mesenchymal stem cell for articular cartilage reconstruction. Curr Stem Cell Res Ther, 2014. 9(3): p. 254-67.
53. Wong, S. P., et al., Pericytes, mesenchymal stem cells and their contributions to tissue repair. Pharmacol Ther, 2015. 151: p. 107-20.
54. Yang, M., H. Zhang, and R. Gangolli, Advances of mesenchymal stem cells derived from bone marrow and dental tissue in craniofacial tissue engineering. Curr Stem Cell Res Ther, 2014. 9(3): p. 150-61.
55. Yum, K., et al., Physiologically relevant organs on chips. Biotechnol J, 2014. 9(1): p. 16-27.
56. Justin, R. T. and A. J. Engler, Stiffness gradients mimicking in vivo tissue variation regulate mesenchymal stem cell fate. PloS one, 2011. 6(1): p. e15978.
57. Vincent, L. G., et al., Mesenchymal stem cell durotaxis depends on substrate stiffness gradient strength. Biotechnology journal, 2013. 8(4): p. 472-484.
58. Dalby, M. J., et al., The control of human mesenchymal cell differentiation using nanoscale symmetry and disorder. Nat Mater, 2007. 6(12): p. 997-1003.
59. Albrecht, D. R., et al., Probing the role of multicellular organization in three-dimensional microenvironments. Nat Methods, 2006. 3(5): p. 369-75.
60. Kern, W., Overview and Evolution of Silicon Wafer Cleaning Technology *, in Handbook of Silicon Wafer Cleaning Technology. 2018. p. 3-85.
61. Proctor, A. and P. M. A. Sherwood, Data analysis techniques in x-ray photoelectron spectroscopy. Analytical Chemistry, 1982. 54(1): p. 13-19.
62. Higo, M., et al., Atomic force microscopy observation of the morphology of tetracyanoquinodimethane (TCNQ) deposited from solution onto the atomically smooth native oxide surface of Al (111) films. Thin Solid Films, 2001. 384(1): p. 90-101.
63. Liu, A., et al., AFM on humic acid adsorption on mica. Colloids and surfaces A: Physicochemical and engineering aspects, 2000. 174(1-2): p. 245-252.
64. Oliver, W. C. and G. M. Pharr, An improved technique for determining hardness and elastic modulus using load and displacement sensing indentation experiments. Journal of materials research, 1992. 7(06): p. 1564-1583.
65. Merchant, F. A., et al., Viability analysis of cryopreserved rat pancreatic islets using laser scanning confocal microscopy. Cryobiology, 1996. 33(2): p. 236-52.
66. Merchant, F. and M. Toner, Spatial and Dynamic Characterization of the Interaction of Staphylococcus Aureus Alpha-Toxin With Cell Membranes. ASME-PUBLICATIONS-HTD, 1997. 355: p. 3-8.
67. Matsuda, T. and T. Sugawara, Control of cell adhesion, migration, and orientation on photochemically microprocessed surfaces. J Biomed Mater Res, 1996. 32(2): p. 165-73.
68. Merchant, F. A., et al., Viability analysis of cryopreserved rat pancreatic islets using laser scanning confocal microscopy. (0011-2240 (Print)).
69. Schneider, C. A., W. S. Rasband, and K. W. Eliceiri, NIH Image to ImageJ: 25 years of image analysis. Nat Methods, 2012. 9(7): p. 671-5.
70. Andrews, S., FastQC: a quality control tool for high throughput sequence data. 2010, Babraham Bioinformatics, Babraham Institute, Cambridge, United Kingdom.
71. Oliveros, J. C. VENNY. An interactive tool for comparing lists with Venn Diagrams. 2007 [cited 2007; //bioinfogp.cnb.csic.es/tools/venny/index.html.
72. Oshlack, A., M. D. Robinson, and M. D. Young, From RNA-seq reads to differential expression results. Genome biology, 2010. 11(12): p. 220.
73. Cho, R. J. and M. J. Campbell, Transcription, genomes, function. Trends Genet, 2000. 16(9): p. 409-15.
74. Mi, H., et al., The PANTHER database of protein families, subfamilies, functions and pathways. Nucleic Acids Research, 2005. 33(suppl 1): p. D284-D288.
75. Garipcan, B., et al., Image Analysis of Endothelial Microstructure and Endothelial Cell Dimensions of Human Arteries—A Preliminary Study. Advanced Engineering Materials, 2011. 13(1-2): p. B54-B57.
76. Skrzypek, K., et al., An important step towards a prevascularized islet macroencapsulation device-effect of micropatterned membranes on development of endothelial cell network. J Mater Sci Mater Med, 2018. 29(7): p. 91.
77. Han, Y., et al., First-principles study of TiB(2)(0001) surfaces. J Phys Condens Matter, 2006. 18(17): p. 4197-205.
78. Volonakis, G., L. Tsetseris, and S. Logothetidis, Electronic and structural properties of TiB2: Bulk, surface, and nanoscale effects. Materials Science and Engineering: B, 2011. 176(6): p. 484-489.
79. Mishra, S. K., P. K. P. Rupa, and L. C. Pathak, Surface and nanoindentation studies on nanocrystalline titanium diboride thin film deposited by magnetron sputtering. Thin Solid Films, 2007. 515(17): p. 6884-6889.
80. Zyganitidis, I., N. Kalfagiannis, and S. Logothetidis, Ultra sharp Berkovich indenter used for nanoindentation studies of TiB2 thin films. Materials Science and Engineering B-Advanced Functional Solid-State Materials, 2009. 165(3): p. 198-201.
81. Dietrich, P. M., et al., XPS depth profiling of an ultrathin bioorganic film with an argon gas cluster ion beam. Biointerphases, 2016. 11(2): p. 029603.
82. Kalina, L., et al., THICKNESS DETERMINATION OF CORROSION LAYERS ON IRON USING XPS DEPTH PROFILING. Materiali in tehnologije, 2018. 52(5): p. 537-540.
83. Oshida, Y., Bioscience and Bioengineering of Titanium Materials. second ed. 2013.
84. Voitovich, V. B., V. A. Lavrenko, and V. M. Adejev, High-temperature oxidation of titanium diboride of different purity. Oxidation of Metals, 1994. 42(1): p. 145-161.
85. Koh, Y. H., H. W. Kim, and H. E. Kim, Improvement in oxidation resistance of TiB2 by formation of protective SiO2 layer on surface. Journal of Materials Research, 2001. 16(1): p. 132-137.
86. Kulpa, A. and T. Troczynski, Oxidation of TiB2 Powders below 900° C. Journal of the American Ceramic Society, 1996. 79(2): p. 518-520.
87. Ong, C. W., et al., X-ray photoemission spectroscopy of nonmetallic materials: Electronic structures of boron and BxOy. Journal of Applied Physics, 2004. 95(7): p. 3527-3534.
88. Ferraris, S., et al., Zeta Potential Measurements on Solid Surfaces for in Vitro Biomaterials Testing: Surface Charge, Reactivity Upon Contact With Fluids and Protein Absorption. Front Bioeng Biotechnol, 2018. 6: p. 60.
89. Sivakumar, B., L. C. Pathak, and R. Singh, Fretting corrosion response of boride coated titanium in Ringer's solution for bio-implant use: Elucidation of degradation mechanism. Tribology International, 2018. 127: p. 219-230.
90. Guan, W., et al., Quantitative probing of surface charges at dielectric-electrolyte interfaces. Lab Chip, 2013. 13(7): p. 1431-6.
91. Okorn-Schmidt, H. F., Characterization of silicon surface preparation processes for advanced gate dielectrics. IBM Journal of Research and Development, 1999. 43(3): p. 351-326.
92. Estrela, P., et al., Label-Free Detection of Protein interactions with peptide aptamers by open circuit potential measurement. Electrochimica Acta, 2008. 53(22): p. 6489-6496.
93. Deligianni, D. D., et al., Effect of surface roughness of the titanium alloy Ti-6Al-4V on human bone marrow cell response and on protein adsorption. Biomaterials, 2001. 22(11): p. 1241-1251.
94. Alter, S. C., et al., Regulation of human mast cell tryptase. Effects of enzyme concentration, ionic strength and the structure and negative charge density of polysaccharides. The Biochemical journal, 1987. 248(3): p. 821-827.
95. Ling, L., et al., Effect of heparin on the biological properties and molecular signature of human mesenchymal stem cells. Gene, 2016. 576(1 Pt 2): p. 292-303. 41
96. Maciag, T., G. A. Hoover, and R. Weinstein, High and low molecular weight forms of endothelial cell growth factor. J Biol Chem, 1982. 257(10): p. 5333-6.
97. Maciag, T., et al., Heparin binds endothelial cell growth factor, the principal endothelial cell mitogen in bovine brain. Science, 1984. 225(4665): p. 932-5.
98. Macleod, A. J., Serum quality: an analysis of its components. Developments in Biological Standardization, 1980. 46: p. 17-20.
99. Bala, K., K. Ambwani, and N. K. Gohil, Effect of different mitogens and serum concentration on HUVEC morphology and characteristics: Implication on use of higher passage cells. Tissue and Cell, 2011. 43(4): p. 216-222.
100. Friedl, P., D. Tatje, and R. Czpla, An optimized culture medium for human vascular endothelial cells from umbilical cord veins. Cytotechnology, 1989. 2(3): p. 171-179.
101. Marin, V., et al., Endothelial cell culture: protocol to obtain and cultivate human umbilical endothelial cells. Journal of Immunological Methods, 2001. 254(1): p. 183-190.
102. Selimovic, S., et al., Microfabricated polyester conical microwells for cell culture applications. Lab on a chip, 2011. 11(14): p. 2325-2332.
103. Vincent, L. G., et al., Mesenchymal stem cell durotaxis depends on substrate stiffness gradient strength. Biotechnol J, 2013. 8(4): p. 472-84.
104. Munoz, E. M. and R. J. Linhardt, Heparin-binding domains in vascular biology. Arteriosclerosis, thrombosis, and vascular biology, 2004. 24(9): p. 1549-1557.
105. Benoit, D. S. and K. S. Anseth, Heparin functionalized PEG gels that modulate protein adsorption for hMSC adhesion and differentiation. Acta Biomater, 2005. 1(4): p. 461-70.
106. Minter, A. J., C. N. Dawes J Fau-Chesterman, and C. N. Chesterman, Effects of heparin and endothelial cell growth supplement on haemostatic functions of vascular endothelium. (0340-6245 (Print)).
107. Zieris, A., et al., FGF-2 and VEGF functionalization of starPEG-heparin hydrogels to modulate biomolecular and physical cues of angiogenesis. Biomaterials, 2010. 31(31): p. 7985-94.
108. Adam Hacking, S. and A. Khademhosseini, Chapter II.1.3—Cells and Surfaces in vitro, in Biomaterials Science (Third Edition), D. R. Buddy, et al., Editors. 2013, Academic Press. p. 408-427.
109. Anderson, D. E. and M. T. Hinds, Endothelial cell micropatterning: methods, effects, and applications. Ann Biomed Eng, 2011. 39(9): p. 2329-45.
110. Li, Y., et al., Engineering cell alignment in vitro. Biotechnology Advances, 2014. 32(2): p. 347-365. 111. Nikkhah, M., et al., Engineering microscale topographies to control the cell-substrate interface. Biomaterials, 2012. 33(21): p. 5230-46.
112. Rizwan, M., et al., Cell-Substrate Interactions, in Principles of Regenerative Medicine. 2019, Elsevier. p. 437-468.
113. Vandrovcova, M. and L. Bacakova, Adhesion, growth and differentiation of osteoblasts on surfacemodified materials developed for bone implants. Physiol Res, 2011. 60(3): p. 403-17.
114. Kaivosoja, E., et al., Enhancement of Silicon Using Micro-Patterned Surfaces of Thin Films. European Cells & Materials, 2010. 19: p. 147-157.
115. Bettinger, C. J., et al., Microfabrication of poly (glycerol-sebacate) for contact guidance applications. Biomaterials, 2006. 27(12): p. 2558-65.
116. Saez, A., et al., Rigidity-driven growth and migration of epithelial cells on microstructured anisotropic substrates. Proc Natl Acad Sci USA, 2007. 104(20): p. 8281-6.
117. Heng, B. C., et al., Effect of cell-seeding density on the proliferation and gene expression profile of human umbilical vein endothelial cells within ex vivo culture. Cytotherapy, 2011. 13(5): p. 606-17.
118. Relou, I. A., et al., Effect of culture conditions on endothelial cell growth and responsiveness. Tissue Cell, 1998. 30(5): p. 525-30.
119. Jain, A., et al., Determination of the thermodynamic stability of TiB2. Journal of Alloys and Compounds, 2010. 491(1-2): p. 747-752.
120. Ma, X., et al., Thermodynamic assessment of the Ti—B system. Journal of Alloys and Compounds, 2004. 370(1-2): p. 149-158.
121. Madtha, S., C. Lee, and K. S. R. Chandran, Physical and mechanical properties of nanostructured titanium boride (TiB) ceramic. Journal of the American Ceramic Society, 2008. 91(4): p. 1319-1321.
122. Munro, R. G., Material properties of titanium diboride. J. Res. Nast. Inst. Stad. Technol., 2000. 105(5): p. 709-720.
123. Masahashi, N. and M. Oku, Superhydrophilicity and XPS study of boron-doped TiO2. Applied Surface Science, 2008. 254(21): p. 7056-7060.
124. Curtis, A. S., M. Dalby, and N. Gadegaard, Cell signaling arising from nanotopography: implications for nanomedical devices. 2006.
125. Stevens, M. M. and J. H. George, Exploring and engineering the cell surface interface. Science, 2005. 310(5751): p. 1135-1138.
126. Brunetti, V., et al., Neurons sense nanoscale roughness with nanometer sensitivity. Proceedings of the National Academy of Sciences, 2010. 107(14): p. 6264.
127. Discher, D. E., P. Janmey, and Y. L. Wang, Tissue cells feel and respond to the stiffness of their substrate. Science, 2005. 310(5751): p. 1139-43.
128. Li, J., D. Han, and Y. P. Zhao, Kinetic behaviour of the cells touching substrate: the interfacial stiffness guides cell spreading. Sci Rep, 2014. 4: p. 3910.
129. Cox, M. N. D. L., Principles of Biochemistry. 2004: Freeman.
130. Dalton, B. A., et al., Role of the heparin binding domain of fibronectin in attachment and spreading of human bone-derived cells. J Cell Sci, 1995. 108 (Pt 5): p. 2083-92.
131. Parsons, J. T., A. R. Horwitz, and M. A. Schwartz, Cell adhesion: integrating cytoskeletal dynamics and cellular tension. Nat Rev Mol Cell Biol, 2010. 11(9): p. 633-643.
132. Mitrossilis, D., et al., Single-cell response to stiffness exhibits muscle-like behavior. Proceedings of the National Academy of Sciences, 2009. 106(43): p. 18243-18248.
133. Khang, D., et al., The role of nanometer and sub-micron surface features on vascular and bone cell adhesion on titanium. Biomaterials, 2008. 29(8): p. 970-83.
134. Mwenifumbo, S., et al., Cell/surface interactions on laser micro-textured titanium-coated silicon surfaces. Journal of Materials Science: Materials in Medicine, 2007. 18(1): p. 9-23.
135. Cyster, L. A., et al., The effect of surface chemistry and nanotopography of titanium nitride (TiN) films on primary hippocampal neurones. Biomaterials, 2004. 25(1): p. 97-107.
136. Llopis-Hernandez, V., et al., Role of surface chemistry in protein remodeling at the cell-material interface. PLoS One, 2011. 6(5): p. e19610.
137. Salloum, D. S., et al., Vascular smooth muscle cells on polyelectrolyte multilayers: hydrophobicity-directed adhesion and growth. Biomacromolecules, 2005. 6(1): p. 161-7.
138. Spriano, S., et al., How do wettability, zeta potential and hydroxylation degree affect the biological response of biomaterials? Mater Sci Eng C Mater Biol Appl, 2017. 74: p. 542-555.
139. Silva-Bermudez, P. and S. E. Rodil, An overview of protein adsorption on metal oxide coatings for biomedical implants. Surface and Coatings Technology, 2013. 233: p. 147-158.
140. Ouberai, M. M., K. Xu, and M. E. Welland, Effect of the interplay between protein and surface on the properties of adsorbed protein layers. Biomaterials, 2014. 35(24): p. 6157-6163.
141. Salis, A., et al., Measurements and Theoretical Interpretation of Points of Zero Charge/Potential of BSA Protein. Langmuir, 2011. 27(18): p. 11597-11604.
142. Anselme, K. and M. Bigerelle, Role of materials surface topography on mammalian cell response. International Materials Reviews, 2011. 56(4): p. 243-266.
143. Borghi, F., et al., Nanoscale roughness and morphology affect the IsoElectric Point of titania surfaces. PLoS ONE, 2013. 8(7): p. e68655.
144. Ranella, A., et al., Tuning cell adhesion by controlling the roughness and wettability of 3D micro/nano silicon structures. Acta Biomater, 2010. 6(7): p. 2711-20.
145. Fenoglio, I., et al., Multiple aspects of the interaction of biomacromolecules with inorganic surfaces. Advanced Drug Delivery Reviews, 2011. 63(13): p. 1186-1209.
146. Rabe, M., D. Verdes, and S. Seeger, Understanding protein adsorption phenomena at solid surfaces. Advances in Colloid and Interface Science, 2011. 162(1): p. 87-106.
147. Bacakova, L., et al., Modulation of cell adhesion, proliferation and differentiation on materials designed for body implants. Biotechnol Adv, 2011. 29(6): p. 739-67.
148. Charest, J. L., et al., Combined microscale mechanical topography and chemical patterns on polymer cell culture substrates. Biomaterials, 2006. 27(11): p. 2487-94.
149. Gentleman, M. M. and E. Gentleman, The role of surface free energy in osteoblast-biomaterial interactions. International Materials Reviews, 2014. 59(8): p. 417-429.
150. Giordano, L., F. Cinquini, and G. Pacchioni, Tuning the surface metal work function by deposition of ultrathin oxide films: Density functional calculations. Physical Review B, 2006. 73(4).
151. Kim, S., A. E. English, and K. D. Kihm, Surface elasticity and charge concentration-dependent endothelial cell attachment to copolymer polyelectrolyte hydrogel. Acta Biomaterialia, 2009. 5(1): p. 144-151.
152. Metwally, S. and U. Stachewicz, Surface potential and charges impact on cell responses on biomaterials interfaces for medical applications. Materials Science and Engineering: C, 2019. 104.
153. Nazneen, F., et al., Surface chemical and physical modification in stent technology for the treatment of coronary artery disease. J Biomed Mater Res B Appl Biomater, 2012. 100(7): p. 1989-2014.
154. Felgueiras, H. P., et al., Fundamentals of protein and cell interactions in biomaterials, in Peptides and Proteins as Biomaterials for Tissue Regeneration and Repair. 2018. p. 1-27.
155. Kubiak-Ossowska, K., et al., Protein interactions with negatively charged inorganic surfaces. Current Opinion in Colloid and Interface Science 2019. 41: p. 104-117.
156. Li, J., et al., Surface Charge Regulation of Osteogenic Differentiation of Mesenchymal Stem Cell on Polarized Ferroelectric Crystal Substrate. Advanced Healthcare Materials, 2015. 4(7): p. 998-1003.
157. Sergeeva, Y. N., et al., What is really driving cell-surface interactions? Layer-by-layer assembled films may help to answer questions concerning cell attachment and response to biomaterials. Biointerphases, 2016. 11(1): p. 019009.
158. Feller, L., et al., Cellular responses evoked by different surface characteristics of intraosseous titanium implants. BioMed research international, 2015. 2015.
159. Georges, P. C. and P. A. Janmey, Cell type-specific response to growth on soft materials. Journal of applied physiology, 2005. 98(4): p. 1547-1553.
160. Lotfi, M., M. Nejib, and M. Naceur, Cell Adhesion to Biomaterials: Concept of Biocompatibility, in Advances in Biomaterials Science and Biomedical Applications, R. Pignatello, Editor. 2013, IntechOpen: Rijeka, Croatia.
161. Lu, X. H., et al., Distinct surface hydration behaviors of boron-rich boride thin film coatings. Applied Surface Science, 2014. 311(0): p. 749-752.
162. Spivak-Kroizman, T., et al., Heparin-induced oligomerization of FGF molecules is responsible for FGF receptor dimerization, activation, and cell proliferation. Cell, 1994. 79(6): p. 1015-1024.
163. Saksela, O., et al., Endothelial cell-derived heparan sulfate binds basic fibroblast growth factor and protects it from proteolytic degradation. The Journal of cell biology, 1988. 107(2): p. 743-751.
164. Jing, F. J., et al., Behavior of endothelial cells on micro-patterned titanium oxide fabricated by plasma immersion ion implantation and deposition and plasma etching. Surface & Coatings Technology, 2007. 201(15): p. 6874-6877.
165. Cipriano, A. F., et al., Bone marrow stromal cell adhesion and morphology on micro- and submicropatterned titanium. J Biomed Nanotechnol, 2014. 10(4): p. 660-8.
166. Yan, C., et al., Synthesis and characterization of hard ternary AlMgB compositefilms prepared bysputter deposition. Thin Solid Films, 2010. 518: p. 5372-5377.
167. Cunningham B C., et al., Dimerization of the extracellular domain of the human growth hormone receptor by a single hormone molecule. Science. 1991. 254(5033): p. 821-5.
168. Gospodarowicz D., et al., Heparin protects basic and acidic FGF from inactivation. J Cell Physiol. 1986. 128(3): p. 475-84.
169. Breithaupt-Faloppa A C., et al., In vitro behaviour of endothelial cells on a titanium surface. Head Face Med. 2008. 4: p. 14.
170. Hasirci V., et al., Understanding the cell behavior on nano-/micro-patterned surfaces. Nanomedicine (Lond). 2012. 7(9): p. 1375-89.
171. Gu et al., “Sorting transition-metal diborides: New descriptor for mechanical properties,” //doi.org/10.1016/j.actamat.2021.116685.
172. Sitler et al., Room Temperature Corrosion Behavior of ZrB₂—HfB₂ Solid Solutions in Acidic and Basic Aqueous Environments,” //dx.doi.org/10.1016/j.electacta.2017.06.033.
173. Parthasarathya et al., “A model for the oxidation of ZrB₂, HfB₂ and TiB₂,” //doi.org/10.1016/j.actamat.2007.07.027.
174. Loehman et al., “Ultra High Temperature Ceramics for Hypersonic Vehicle Applications,” SANDIA REPORT, SAND 2006-2925, Unlimited Release, June 2006.
175. Zagozdzon-Wosik et al., “Applications of metallic borides for gate electrodes in CMOS integrated circuits,” Rev. Adv. Mat. Sci. 8, 2004, 185-194.
176. Rnjit et al., “Formation of contacts and integration with shallow junctions using diborides of Ti, Zr, and Hf,” Rev. Adv. Mat. Sci. 8, 2004, 176-184.
177. Zagozdzon-Wosik et al., “Microstructure and electrical properties of diborides modified by rapid thermal annealing,” Journal of Microscopy, Vol. 223, Pt 3 Sep. 2006, pp. 227-230.
178. Mohammadi et al., “Tissue response to hafnium,” J Mater Sci Mater Med. 2001 July; 12(7):603-11.
179. Kaya et al., “Microstructure characterization and biocompatibility behaviour of TiNbZr alloy fabricated by powder metallurgy,” //doi.org/10.1088/2053-1591/ab58a5.
180. Liva et al., α′-Type Ti—Nb—Zr alloys with ultra-low Young's modulus and high strength,” //dx.doi.org/10.1016/j.pnsc.2013.11.005.
181. Gonzalez et al., “Low modulus Ti—Nb—Hf alloy for biomedical applications,” //dx.doi.org/10.1016/j.msec.2014.06.010.
182. Wang et al., “In vitro cytotoxicity and hemocompatibility studies of Ti—Nb, Ti—Nb—Zr and Ti—Nb—Hf biomedical shape memory alloys,” doi:10.1088/1748-6041/5/4/044102.
183. Passerone, et al., “Wetting of Group IV diborides by liquid metals. J Mater Sci 41, 5088-5098 (2006).
184. Wang et al., “First-principles study on the stability and work function of low-index surfaces of TiB2,” Computational Materials Science, Volume 172, 2020, 109356.
185. Poilov et al., “Thermodynamics of Oxidation of Zirconium and Hafnium Borides,” ISSN 0036-0236, Russian Journal of Inorganic Chemistry, 2016, Vol. 61, No. 1, pp. 55-58.
186. Kosmulski, M., “Isoelectric points and points of zero charge of metal (hydr)oxides: 50 years after Parks' review,” Advances in Colloid and Interface Science,” Volume 238, 2016, Pages 1-61.
187. He et al., “Surface preoxidation to improve dispersibility of zirconium diboride in aqueous medium,” Advances in Applied Ceramics, 113:5, 311-314, 2014.
188. Torre L A, Trabert B, DeSantis C E, Miller K D, Samimi G, Runowicz C D, Gaudet Jemal A, Siegel R L. Ovarian cancer statistics, 2018. CA Cancer J Clin. 2018; 68(4):284-96. Epub 2018/05/29. doi: 10.3322/caac.21456. PubMed PMID: 29809280; PMCID: PMC6621554.
189. Davidson B, Trope C G, Reich R. Epithelial-mesenchymal transition in ovarian carcinoma. Front Oncol. 2012; 2:33. Epub 2012/06/02. doi: 10.3389/fonc.2012.00033. PubMed PMID: 22655269; PMCID: PMC3356037.
190. Padilla M A A, Binju M, Wan G, Rahmanto Y S, Kaur P, Yu Y. Relationship between ovarian cancer stem cells, epithelial mesenchymal transition and tumour recurrence. Cancer Drug Resistance. 2019; 2(4):1127-35. doi: 10.20517/cdr.2019.76.
191. Huang H-K, Lin Y-H, Chang H-A, Lai Y-S, Chen Y-C, Huang S-C, Chou C-Y, Chiu W-T. Chemoresistant ovarian cancer enhances its migration abilities by increasing store-operated Ca2+ entry-mediated turnover of focal adhesions. Journal of Biomedical Science. 2020; 27(1):36. doi: 10.1186/s12929-020-00630-5.
192. Nuti S V, Mor G, Li P, Yin G. TWIST and ovarian cancer stem cells: implications for chemoresistance and metastasis. Oncotarget. 2014; 5(17):7260-71. Epub 2014/09/23. doi: 10.18632/oncotarget.2428. PubMed PMID: 25238494; PMCID: PMC4202121.
193. Shieh A C. Biomechanical Forces Shape the Tumor Microenvironment. Annals of Biomedical Engineering. 2011; 39(5):1379-89. doi: 10.1007/s10439-011-0252-2.
194. Seo Y H, Jo Y-n, Oh Y J, Park S. Nano-mechanical Reinforcement in Drug-Resistant Ovarian Cancer Cells. Biological and Pharmaceutical Bulletin. 2015; 38(3):389-95. doi: 10.1248/bpb.b14-00604.
195. Deng J, Wang L, Chen H, Hao J, Ni J, Chang L, Duan W, Graham P, Li Y. Targeting epithelial-mesenchymal transition and cancer stem cells for chemoresistant ovarian cancer 2016(1949-2553 (Electronic)).
196. Horowitz M, Esakov E, Rose P, Reizes O. Signaling within the epithelial ovarian cancer tumor microenvironment: the challenge of tumor heterogeneity. Ann Transl Med. 2020; 8(14):905-. doi: 10.21037/atm-2019-cm-08. PubMed PMID: 32793749.
197. Kim S, Han Y, Kim S I, Kim H-S, Kim S J, Song Y S. Tumor evolution and chemoresistance in ovarian cancer. npj Precision Oncology. 2018; 2(1):20. doi: 10.1038/s41698-018-0063-0.
198. Arantes-Rodrigues R, ColaÇO A, Pinto-Leite R, Oliveira P A. In Vitro and In Vivo Experimental Models as Tools to Investigate the Efficacy of Antineoplastic Drugs on Urinary Bladder Cancer. Anticancer Research. 2013; 33(4):1273.
199. Zanoni M, Piccinini F, Arienti C, Zamagni A, Santi S, Polico R, Bevilacqua A, Tesei A. 3D tumor spheroid models for in vitro therapeutic screening: a systematic approach to enhance the biological relevance of data obtained. Scientific Reports. 2016; 6(1):19103. doi: 10.1038/srep19103.
200. Friguglietti J, Das S, Le P, Fraga D, Quintela M, Gazze S A, McPhail D, Gu J, Sabek O, Gaber A O, Francis L W, Zagozdzon-Wosik W, Merchant F A. Novel Silicon Titanium Diboride Micropatterned Substrates for Cellular Patterning. Biomaterials. 2020; 244:119927. doi: 10.1016/j.biomaterials.2020.119927. PubMed PMID: 32199283.
201. McPhail D. Titanium diboride bioMEMS; investigating ovarian cancer spheroid formation in the presence of epigenomic instability. Swansea, Wales: Swansea University; 2019.
202. Lele T P, Sero J E, Matthews B D, Kumar S, Xia S, Montoya-Zavala M, Polte T, Overby D, Wang N, Ingber D E. Tools to Study Cell Mechanics and Mechanotransduction. Methods in Cell Biology: Academic Press; 2007. p. 441-72.
203. Moon J J, Saik J E, Poché RA, Leslie-Barbick J E, Lee S-H, Smith A A, Dickinson M E, West J L. Biomimetic hydrogels with pro-angiogenic properties. Biomaterials. 2010; 31(14):3840-7. doi: https://doi.org/10.1016/j.biomaterials.2010.01.104.
204. Heredia-Soto V, Redondo A, Berjón A, Miguel-Martin M, Diaz E, Crespo R, Hernandez A, Yébenes L, Gallego A, Feliu J, Hardisson D, Mendiola M. High-throughput 3-dimensional culture of epithelial ovarian cancer cells as preclinical model of disease. Oncotarget. 2018; 9(31):21893-903. doi: 10.18632/oncotarget.25098. PubMed PMID: 29774110.
205. Inoue A, Deem A K, Kopetz S, Heffernan T P, Draetta G F, Carugo A. Current and Future Horizons of Patient-Derived Xenograft Models in Colorectal Cancer Translational Research. Cancers (Basel). 2019; 11(9):1321. doi: 10.3390/cancers11091321. PubMed PMID: 31500168.
206. Langhans S A. Three-Dimensional in Vitro Cell Culture Models in Drug Discovery and Drug Repositioning. Frontiers in Pharmacology. 2018; 9(6). doi: 10.3389/fphar.2018.00006.
207. Shamir E R, Ewald A J. Three-dimensional organotypic culture: experimental models of mammalian biology and disease. Nature reviews Molecular cell biology. 2014; 15(10):647-64. Epub 2014/09/23. doi: 10.1038/nrm3873. PubMed PMID: 25237826; PMCID: PMC4352326.
208. Xu X, Farach-Carson M C, Jia X. Three-dimensional in vitro tumor models for cancer research and drug evaluation. Biotechnol Adv. 2014; 32(7):1256-68. Epub 2014/08/10. doi: 10.1016/j.biotechadv.2014.07.009. PubMed PMID: 25116894.
209. Huang R Y, Wong M K, Tan T Z, Kuay K T, Ng A H, Chung V Y, Chu Y S, Matsumura N, Lai H C, Lee Y F, Sim W J, Chai C, Pietschmann E, Mori S, Low J J, Choolani M, Thiery J P. An EMT spectrum defines an anoikis-resistant and spheroidogenic intermediate mesenchymal state that is sensitive to e-cadherin restoration by a src-kinase inhibitor, saracatinib (AZD0530). Cell Death Dis. 2013; 4:e915. Epub 2013/11/10. doi: 10.1038/cddis.2013.442. PubMed PMID: 24201814; PMCID: PMC3847320.
210. Hernandez L, Kim M K, Lyle L T, Bunch K P, House C D, Ning F, Noonan A M, Annunziata C M. Characterization of ovarian cancer cell lines as in vivo models for preclinical studies. Gynecol Oncol. 2016; 142(2):332-40. Epub 2016/05/29. doi: 10.1016/j.ygyno.2016.05.028. PubMed PMID: 27235858; PMCID: PMC4961516.
211. Shaw T J, Senterman M K, Dawson K, Crane C A, Vanderhyden B C. Characterization of intraperitoneal, orthotopic, and metastatic xenograft models of human ovarian cancer. Mol Ther. 2004; 10(6):1032-42. Epub 2004/11/27. doi: 10.1016/j.ymthe.2004.08.013. PubMed PMID: 15564135.
212. Dobin A, Davis C A, Schlesinger F, Drenkow J, Zaleski C, Jha S, Batut P, Chaisson M, Gingeras T R. STAR: ultrafast universal RNA-seq aligner. Bioinformatics. 2013; 29(1):15-21. Epub 2012/10/30. doi: 10.1093/bioinformatics/bts635. PubMed PMID: 23104886; PMCID: PMC3530905.
213. Subramanian A, Tamayo P, Mootha V K, Mukherjee S, Ebert B L, Gillette M A, Paulovich A, Pomeroy S L, Golub T R, Lander E S, Mesirov J P. Gene set enrichment analysis: A knowledge-based approach for interpreting genome-wide expression profiles. Proceedings of the National Academy of Sciences. 2005; 102(43):15545. doi: 10.1073/pnas.0506580102.
214. Freeman J, Smith D, Latinkic B, Ewan K, Samuel L, Zollo M, Marino N, Tyas L, Jones N, Dale T C. A functional connectome: regulation of Wnt/TCF-dependent transcription by pairs of pathway activators. Mol Cancer. 2015; 14:206. Epub 2015/12/09. doi: 10.1186/s12943-015-0475-1. PubMed PMID: 26643252; PMCID: PMC4672529.
215. Porter A P, Papaioannou A, Malliri A. Deregulation of Rho GTPases in cancer. Small GTPases. 2016; 7(3):123-38. doi: 10.1080/21541248.2016.1173767.
216. Yano M, Yasuda M, Sakaki M, Nagata K, Fujino T, Arai E, Hasebe T, Miyazawa M, Miyazawa M, Ogane N, Hasegawa K, Narahara H. Association of histone deacetylase expression with histology and prognosis of ovarian cancer. Oncol Lett. 2018; 15(3):3524-31. Epub 2018/02/20. doi: 10.3892/01.2018.7726. PubMed PMID: 29456726; PMCID: PMC5795841.
217. Han H, Yang B, Nakaoka H J, Yang J, Zhao Y, Le Nguyen K, Bishara A T, Mandalia T K, Wang W. Hippo signaling dysfunction induces cancer cell addiction to YAP. Oncogene. 2018; 37(50):6414-24. Epub 2018/08/03. doi: 10.1038/s41388-018-0419-5. PubMed PMID: 30068939; PMCID: PMC6294669.
218. Zhang A W, McPherson A, Milne K, Kroeger D R, Hamilton P T, Miranda A, Funnell T, Little N, de Souza C P E, Laan S, LeDoux S, Cochrane D R, Lim J L P, Yang W, Roth A, Smith M A, Ho J, Tse K, Zeng T, Shlafman I, Mayo M R, Moore R, Failmezger H, Heindl A, Wang Y K, Bashashati A, Grewal D S, Brown S D, Lai D, Wan A N C, Nielsen C B, Huebner C, Tessier-Cloutier B, Anglesio M S, Bouchard-Cote A, Yuan Y, Wasserman W W, Gilks C B, Karnezis A N, Aparicio S, McAlpine J N, Huntsman D G, Holt R A, Nelson B H, Shah S P. Interfaces of Malignant and Immunologic Clonal Dynamics in Ovarian Cancer. Cell. 2018; 173(7):1755-69.e22. Epub 2018/05/15. doi: 10.1016/j.cell.2018.03.073. PubMed PMID: 29754820.
219. Konstantinopoulos P A, Wilson A J, Saskowski J, Wass E, Khabele D. Suberoylanilide hydroxamic acid (SAHA) enhances olaparib activity by targeting homologous recombination DNA repair in ovarian cancer. Gynecologic Oncology. 2014; 133 (3): 599-606. doi: https://doi.org/10.1016/j.ygyno.2014.03.007.
220. Wang Y, Chen S Y, Colborne S, Lambert G, Shin C Y, Santos N D, Orlando K A, Lang J D, Hendricks W P D, Bally M B, Karnezis A N, Hass R, Underhill T M, Morin G B, Trent J M, Weissman B E, Huntsman D G. Histone Deacetylase Inhibitors Synergize with Catalytic Inhibitors of EZH2 to Exhibit Antitumor Activity in Small Cell Carcinoma of the Ovary, Hypercalcemic Type. Molecular Cancer Therapeutics. 2018; 17(12):2767. doi: 10.1158/1535-7163.MCT-18-0348.
221. Li J, Olson L M, Zhang Z, Li L, Bidder M, Nguyen L, Pfeifer J, Rader J S. Differential display identifies overexpression of the USP36 gene, encoding a deubiquitinating enzyme, in ovarian cancer. Int J Med Sci. 2008; 5(3):133-42. doi: 10.7150/ijms.5.133. PubMed PMID: 18566677.
222. Wang W, Wang J, Yan H, Zhang K, Liu Y. Upregulation of USP11 promotes epithelial-to-mesenchymal transition by deubiquitinating Snail in ovarian cancer. Oncol Rep. 2019; 41(3):1739-48. doi: 10.3892/or.2018.6924.
223. Kongsema M, Zona S, Karunarathna U, Cabrera E, Man E P, Yao S, Shibakawa A, Khoo U S, Medema R H, Freire R, Lam E W. RNF168 cooperates with RNF8 to mediate FOXM1 ubiquitination and degradation in breast cancer epirubicin treatment. Oncogenesis. 2016; 5(8):e252. Epub 2016/08/16. doi: 10.1038/oncsis.2016.57. PubMed PMID: 27526106; PMCID: PMC5007831.
224. Vogel R I, Pulver T, Heilmann W, Mooneyham A, Mullany S, Zhao X, Shahi M, Richter J, Klein M, Chen L, Ding R, Konecny G, Kommoss S, Winterhoff B, Ghebre R, Bazzaro M. USP14 is a predictor of recurrence in endometrial cancer and a molecular target for endometrial cancer treatment. Oncotarget. 2016; 7(21):30962-76. Epub 2016/04/29. doi: 10.18632/oncotarget.8821. PubMed PMID: 27121063; PMCID: PMC5058731.
225. Jin C, Yu W, Lou X, Zhou F, Han X, Zhao N, Lin B. UCHL1 Is a Putative Tumor Suppressor in Ovarian Cancer Cells and Contributes to Cisplatin Resistance. J Cancer. 2013; 4(8):662-70. Epub 2013/10/25. doi: 10.7150/j ca.6641. PubMed PMID: 24155778; PMCID: PMC3805994.
226. Wang L, Chen T, Li X, Yan W, Lou Y, Liu Z, Chen H, Cui Z. USP39 promotes ovarian cancer malignant phenotypes and carboplatin chemoresistance. Int J Oncol. 2019; 55(1):277-88. Epub 2019/06/11. doi: 10.3892/ijo.2019.4818. PubMed PMID: 31180526.
227. Coughlin K, Anchoori R, Iizuka Y, Meints J, MacNeill L, Vogel R I, Orlowski R Z, Lee M K, Roden R B, Bazzaro M. Small-molecule RA-9 inhibits proteasome-associated DUBs and ovarian cancer in vitro and in vivo via exacerbating unfolded protein responses. Clin Cancer Res. 2014; 20(12):3174-86. Epub 2014/04/15. doi: 10.1158/1078-0432.CCR-13-2658. PubMed PMID: 24727327; PMCID: PMC4269153.
228. Beaufort C M, Helmijr J C A, Piskorz A M, Hoogstraat M, Ruigrok-Ritstier K, Besselink N, et al. Ovarian cancer cell line panel (OCCP): Clinical importance of in vitro morphological subtypes. PLoS ONE. 2014 Sep. 17; 9(9).

1. A composition comprising a patterned surface, said patterned surface comprising:
(a) a silicon-containing substrate (Si/SiO₂); and
(b) diboride patterned on said silicon substrate,
wherein said patterned surface comprises both silicon and diboride exposed portions.

2. The composition of claim 1, wherein said patterned surface further is exposed to one or more biological molecules, and thereby comprises of adsorbed biological molecules.

3. The composition of claim 2, wherein said one or more biological molecules comprise heparin, endothelial cell growth supplement (ECGS), fibroblast growth factor (FGF), insulin-like growth factor-1 (IGF-1), vascular endothelial growth factor (VEGF), endothelial growth factor (EGF), and/or any protein with a heparin-binding domain (e.g., vitronectin, fibronectin).

4. The composition of claim 2, wherein said one or more biological molecules comprise endothelial cell growth supplement (ECGS), fetal bovine serum (FBS) and heparin, and/or heparin binding proteins, fetal bovine serum (FBS) and heparin.

5. The composition of claim 1, wherein said patterned surface comprises one or more TiB₂ exposed zones surrounded by exposed silicon regions.

6. The composition of claim 5, wherein said one or more diboride exposed zones is 1, 2, 3, 4, 5, 6, 7, 8, 9, 10, 15, 20, 25, 30, 35, 40, 45, 50 75, 100, 150, 200, 250, 500, 750, 1000, 2000, 3000, 4000, 5000, 7500 or 10,000 TiB₂ exposed zones.

7. The composition of claim 5, wherein one or more TiB₂ zones comprises zones of 50 to 1000 μM, such as in the form of lines, circles, squares, rectangles, ovals, and/or any geometric shapes.

8. The composition of claim 1, wherein said patterned surface enables a 3D microenvironment via cell aggregation.

9. The composition of claim 1, wherein said patterned surface is located in a microwell, on a slide, chip or wafer, tissue culture flasks, and/or any other conventional tissue culture containers.

10. The composition of claim 2, wherein said patterned surface comprises ECGS+heparin, FBS+heparin, FBS+ECGS+heparin, and/or FBS+heparin+any heparin binding protein.

11. The composition of claim 1, wherein the diboride is TiB₂, ZrB₂ or HfB₂.

12. A method for capturing and/or culturing a cell comprising contacting a cell or cell-containing composition with a composition of claim 1.

13. The method of claim 12, wherein said cells are endothelial cells, (e.g., HUVECs), cancer cells (e.g., SKOV3, OVCAR3), mesenchymal stem cells (MSCs), any cells of epithelial and/or endothelial lineage and mesodermal lineage, and non-aggressive and/or aggressive cancer cells, such as ovarian or breast cancer cells, and their combinations (i.e., co-culture of different cell types).

14. The method of claim 12, further comprising measuring a functional, surface or structural parameter of cell biology.

15. The method of claim 14, wherein said functional, surface or structural parameter is growth, migration, division, gene expression, surface biomarkers, viability, microskeletal state, oxidative respiration, metastatic potential, apoptosis, biomechanical forces, secretome, and/or transcriptome.

16. The method of claim 12, further comprising treating said cell with a drug, biologic, light, heat or radiation.

17. The method of claim 16, further comprising measuring said functional or structural parameter of cell biology a second time.
